(19) United States
(12) Patent Application Publication
(10) Pub. No.: US 2013/0259842 A1
(43) Pub. Date: Oct. 3, 2013

(54) STABLE REPROGRAMMED CELLS

(71) Applicants: Lee Rubin, Wellesley, MA (US); Joel Blanchard, Pownal, ME (US); Kelvin Lam, Arlington, MA (US)

(72) Inventors: Lee Rubin, Wellesley, MA (US); Joel Blanchard, Pownal, ME (US); Kelvin Lam, Arlington, MA (US)

(21) Appl. No.: 13/698,170

(22) PCT Filed: May 18, 2011
(86) PCT No.: PCT/US11/36986

§ 371 Date: Jun. 10, 2013

Related U.S. Application Data
(60) Provisional application No. 61/345,948, filed on May 18, 2010.
(60) Provisional application No. 61/356,468, filed on Jun. 18, 2010.

(51) Int. Cl. C12N 5/071; C12N 5/0793; C12N 15/85; A61K 35/54; C12Q 1/68

(52) U.S. Cl. CPC C12N 5/0696; A61K 35/545; C12Q 1/6809; C12N 15/85; C12N 5/0619; C12N 5/0657
USPC 424/93.21; 435/325; 424/93.7; 435/6.12

(57) ABSTRACT

The present invention relates to a stable pluripotent reprogrammed cells, and compositions and methods of isolation and uses thereof, wherein the stable pluripotent reprogrammed cells is derived from a somatic cell and has undergone incomplete remodeling of the epigenome. In some embodiments, the stable reprogrammed cell is a human stable reprogrammed cell. In some embodiments, the stable reprogrammed cell has a statistically significant lower level of expression of one or any combination of Nanog, Dnmt3b, Lefty2 as compared to an induced pluripotent stem cell. In some embodiments, the stable reprogrammed cell has a statistically significant higher level of expression of one or any combination of Tdgf1, Tert or endogenous Sox2 as compared to a somatic cell from which it was derived. In some embodiments, the stable reprogrammed cell has a statistically significant faster rate of doubling as compared to an induced pluripotent stem cell (iPSC) or an embryonic stem (ES) cell. Other aspects of the invention relate to compositions comprising the reprogrammed cell, method of isolation and method of use.

### CROSS REFERENCE TO RELATED APPLICATIONS

This application claims benefit under 35 U.S.C. 119(e) of U.S. Provisional Patent Application Ser. No. 61/345,948 filed on May 18, 2010, and U.S. Provisional Patent Application Ser. No. 61/356,468 filed on Jun. 18, 2010, the contents of each are incorporated herein in their entity by reference.

### FIELD OF THE INVENTION

The present invention relates to compositions comprising at least one reprogrammed cell derived from a somatic cell, whereby the reprogrammed cell can be reprogrammed further. The present invention relates to uses of a plurality of different types of reprogrammed cells, methods of reprogramming and uses thereof, in drug screening and use for in vitro and in vivo disease modeling.

### BACKGROUND OF THE INVENTION

One goal of regenerative medicine is to be able to convert an adult differentiated cell into other cell types for tissue repair and regeneration. Retroviral transduction with three genes: Sox2, Oct4, and Klf4, has been shown to completely reprogram mouse or human differentiated cells (e.g. somatic cells) to a pluripotent stem cell state. Mouse and human somatic cells are reprogrammed to a pluripotent state via ectopic expression of KLF4, OCT4, SOX2, and cMYC. Induced pluripotent stem cells (iPSC) may serve as a valuable source of patient or disease-specific tissue for transplantation therapies or disease modeling and drug screening (Rubin et al., 2009). However, converting any cell to pluripotency via defined factor reprogramming remains a slow and inefficient process (Hanna et al., 2009). Defined factor reprogramming is a gradual process that follows a defined series of molecular and epigenetic changes (Brambrink et al., 2008; Stadtfeld et al., 2008).

Unfortunately, the resulting induced pluripotent stem (iPS) cells are suboptimal for uses in transplantation medicine and disease modeling because they have undergone complete remodeling of the epigenome and therefore are disadvantageous for studying disease phenotypes that are encoded in the epigenetic memory of a cell. Furthermore, as the cells may not differentiate efficiently along a particular desired lineage and present risks of spontaneously forming teratomas and tumor formation.

In view of these problems, there remains a need in the art for reprogramming a cell without losing the epigenetic memory of the cell, leading to a better safety profile in therapeutic treatment. In addition, cells that retain the epigenetic memory of a diseased state are useful for the development of disease models when differentiated to a clinically relevant cell type which is a valuable resource for disease modeling and therapeutic screening.

### SUMMARY OF THE INVENTION

Retroviral transduction with Klf4, Sox2, Oct4, and cMyc reprograms murine somatic cells to a pluripotent state. However, the process of reprogramming remains inefficient. The majority of transduced cells fail to fully reprogram, sometimes unable to progress past a partially reprogrammed state. Although these partially reprogrammed cells readily accumulate during viral reprogramming, their stability, pluripotency, molecular and epigenetic characteristics have yet to be fully described. The inventors have determined that stable partially reprogrammed cells are produced and that these stable cells cab be isolated and passaged in culture, and comprise unique characteristics as compared to fully reprogrammed cells. Additionally, the stable partially reprogrammed cells as disclosed herein can be further reprogrammed.

To further characterize these stable partially reprogrammed cells, Oct4::GFP murine embryonic fibroblasts were transduced with Klf4, Sox2, Oct4, and cMyc, and three weeks post infection, partially reprogrammed cell colonies were isolated to establish multiple partially reprogrammed iPS (piPS) cell lines. Stable piPS cell lines formed colonies with distinct morphologies capable of self-renewing for greater than twenty passages. piPS cell lines were discovered to express lower levels of endogenous Oct4, Sox2, Nanog, Rex1, Tdgf1 and other marker of pluripotency as compared to mESC or iPSC. piPSC lines were also discovered to express lower levels of fibroblast markers such as Krt10 and Thy1 as compared to MEFs. This demonstrates that piPSC lines are in an intermediate molecular state distinct from the parent fibroblasts or fully reprogrammed iPSC. Intermediate states of reprogramming are also characterized by incomplete epigenetic reprogramming. The level of Dnmt3b expression in piPSC cell lines was determined to be approximately at least 15-fold lower as compared to completely reprogrammed lines (e.g., iPSC lines). Additionally, the 5′UTR of Oct4 and Nanog in piPSC cell lines exhibited significantly higher levels of CpG methylation than fully reprogrammed (iPSC) lines. In addition, female piPSC lines actively transcribed Xist demonstrating a failure to reactivate silenced X-chromosomes.

Although piPSC lines exhibit incomplete epigenetic and molecular reprogramming, they were discovered to be capable of being more further reprogrammed to completely reprogrammed cells (e.g., by being treated with modulators of DNA methylation, chromatin structure or particular signal transduction pathways) and/or differentiating along particular cell lineages. Upon LIF withdrawal, piPS cell lines form embryoid bodies containing cells that stain positive for Sox17 (endodermal lineage), skeletal muscle myosin (mesodermal lineage) and TUJ1 (ectodermal lineage), demonstrating that piPS cell lines are capable of multilineage differentiation. Furthermore, piPS lines can be directed to differentiate into Hb9+, TUJ1+ motor neurons, or alpha-actinin+ beating cardiac myocytes at rates similar to mES cells or bona fide iPS cells. Collectively, this work demonstrates that during the process of viral reprogramming, a subset of cells become trapped in a self-renewing, virally induced, bimodal state, capable or multi-lineage differentiation or complete reprogramming. The inventors also performed molecular, epigenetic, and in vivo differentiation studies to further characterize and demonstrate the potential of intermediate reprogrammed states.

The inventors have used retroviral transduction with Klf4, Sox2, Oct4 and cMyc to reprogram human and murine somatic cells to a pluripotent state. Herein, using the small molecule substitutes for the reprogramming factors, the inventors have discovered the appearance of intermediate, partially reprogrammed, cells. These intermediate, partially reprogrammed cells, which have not been previously isolated, have unique characteristics as compared to fully reprogrammed cells. Previously, these partially reprogrammed cells were discarded as it was considered they were not suitable for use as they were not fully reprogrammed iPSCs.

To further characterize these stable partially reprogrammed cell types, three weeks post transfection of Oct4:GFP murine embryonic fibroblasts with Klf4, Sox2, Oct4 and cMyc, multiple partially reprogrammed iPS cell (piPSC) lines were isolated. One characteristic of these stable partially reprogrammed cell lines is that they are stable reprogrammed cells which are pluripotent, yet they can also be further reprogrammed to terminally and fully induced pluripotent stem cells (iPSCs).

The inventors demonstrate that clonally expanded piPSCs form colonies have distinct morphologies as compared to iPSC cell colonies, and are capable of self-renewing for greater than twenty passages. The inventors also demonstrate that there is heterogeneity between piPSC lines, and surprisingly, all lines co-express lower levels of endogenous Oct4, Sox2, Nanog, Rex1, Tdgf1 and other markers of pluripotency as compared to either mES cells or isogenic iPS cells. At the same time, piPS cells express lower levels of fibroblast markers such as Thy1 than MEFs from which the piPS cells were reprogrammed from.

The stable reprogrammed cells or “piPSCs” are a cell in an intermediate state of reprogramming, and in some embodiments, can be characterized by incomplete epigenetic reprogramming. For example, the inventors have discovered that the level of Dnmt3b expression in piPSCs is approximately about 15-fold lower compared to completely reprogrammed lines, e.g., iPS cell lines. Additionally, the inventors have demonstrated that the 5′UTR of Oct4 and Nanog in piPSCs exhibit significantly higher levels of CpG methylation than fully reprogrammed lines, e.g., iPSCs. In addition, female piPSC lines actively transcribe Xist, demonstrating an incomplete reactivation of silenced X-chromosomes. In all, the inventors have demonstrated that piPSCs are a stable intermediate reprogrammed cell, which are in a heterogeneous intermediate molecular state, and can be further reprogrammed to fully reprogrammed cells (e.g., iPSCs). The inventors further demonstrate that piPSCs are distinct from both parental fibroblasts from which they are derived or reprogrammed from, and fully reprogrammed iPS cells.

Additionally, the inventors have demonstrated that although piPS cells exhibit incomplete epigenetic and molecular reprogramming, the stable reprogrammed cells, e.g., piPS are still either capable of being more completely reprogrammed (e.g., by being treated with modulators of DNA methylation, chromatin structure or particular signal transduction pathways) or are capable of differentiation along different cell lineages. The inventors herein demonstrate that piPS cell lines are capable of forming embryoid bodies (EBs) in vitro, or teratomas in vivo that contain cells from each embryonic germ-layer. Furthermore, the inventors have demonstrated that a plurality of different piPSC lines can be generated, each with distinct molecular signatures, and can be directed to differentiate into Hb9+, TUJ1+ motor neurons, or alpha-actinin+ beating cardiac myocytes at rates similar to mES cells or bona fide fully-reprogrammed iPS cells. Accordingly, the inventors have demonstrated that reprogramming a population of somatic cells results in a subset of cells which become trapped in a self-renewing metastable state, which are capable of multi-lineage differentiation or complete reprogramming. In the past, after a procedure to reprogram a population of somatic cells, (e.g., by retroviral transduction with Klf4, Sox2, Oct4 etc) the cells which had not fully reprogrammed were discarded, as the art considered that only fully reprogrammed cells to be pluripotent and only those fully reprogrammed cells were of any value for differentiation into different cell lines, or for use in therapeutic methods and/or drug development and screening assays. Thus, herein the inventors have demonstrated the unexpected properties of partially or incompletely reprogrammed (e.g., not fully reprogrammed) cells which are a stable population of pluripotent cells which have a high efficiency for multi-lineage differentiation into neuronal and muscle lineages.

piPSCs as disclosed herein are useful in assays, for example, in disease modeling, since they can be produced rapidly and relatively efficiently and are epigenetically more similar to the cells from which they are derived. The inventors have used the piPSCs as disclosed herein in assays for assessment of molecular, epigenetic, and differentiation studies in further characterizing and determining the potential of intermediate reprogrammed states.

piPSC lines exhibit heterogeneous expression of early reprogramming markers and distinct proliferative rates. To characterize differences between piPSC and iPSC, the inventors first immunostained for the early reprogramming markers alkaline phosphatase (AP) and SSEA1. Using these markers, piPSC lines were categorized into three expression and morphological profiles of the early reprogramming markers SSEA1 and AP.

One aspect of the present invention relates to an isolated reprogrammed cell induced from a differentiated cell, wherein the cell has been reprogrammed to a less differentiated state and can form colonies with distinct morphologies and is capable of self renewing for at least twenty passages before senescence. In some embodiments, the isolated reprogrammed cell can differentiate into all three primary germ layer lineages selected from; endoderm lineage, mesoderm lineage and ectoderm lineage.

In some embodiments, the isolated reprogrammed cell has a doubling time of less than 15 hours, for example between 15 and 5 hours, and in some embodiments, a reprogrammed cell has a significantly faster rate of doubling time as compared to a completely reprogrammed iPS cell, for example, a doubling time of at least 1.5 fold faster as compared to an induced pluripotent stem (iPS) cell, or at least about 2-fold faster as compared to an induced pluripotent stem (iPS) cell.

In some embodiments, an isolated stable reprogrammed cell has a lower expression of Dnmt3b by a statistically significant amount relative to the level of expression of an induced pluripotent stem (iPS) cell, such as at least a 100-fold lower expression, or at least a 200-fold lower expression, or at least a 500-fold lower expression of Dnm3b as compared to the level of expression of an induced pluripotent stem (iPS) cell.

In some embodiments, an isolated stable reprogrammed cell has a lower expression of at least 2 of the following genes; endogenous Oct4, endogenous Nanog, endogenous Rex1, endogenous Tdgf1 by a statistically significant amount relative to the level of expression of an induced pluripotent stem (iPS) cell.

In some embodiments, an isolated stable reprogrammed cell has at least a 100-fold lower expression of endogenous Nanog as compared to the level of expression of an induced pluripotent stem (iPS) cell, for example, at least a 1000-fold lower expression, or at least a 5000-fold lower expression, or about or more than a 10,000-fold lower expression of endogenous Nanog as compared to the level of expression of an induced pluripotent stem (iPS) cell.

In some embodiments, an isolated stable reprogrammed cell has a lower expression of endogenous Lefty 2 by a statistically significant amount relative to the level of expression of an induced pluripotent stem (iPS) cell, for example, at least a 100-fold lower expression, or at least a 1000-fold lower expression, or at least a 10,000-fold lower expression, or anywhere between about 15,000-fold and 10,000-fold lower expression of endogenous Lefty 2 as compared to the level of expression of an induced pluripotent stem (iPS) cell.

In some embodiments, an isolated stable reprogrammed cell has a higher level of expression of any one or more of endogenous Tdgf1, endogenous Tert or endogenous Sox2 by a statistically significant amount relative to the level of expression of a somatic cell, for example the somatic cell from which it was derived.

In some embodiments, an isolated stable reprogrammed cell has at least a 1,000-fold higher expression of endogenous Tgdf1 as compared to the level of expression of endogenous Tgdf1 in a somatic cell, for example, at least a 5,000-fold higher expression, or at least about a 7,000-fold higher expression, or anywhere between about a 7,000-fold to 15,000-fold higher expression of endogenous Tgdf1 as compared to the level of expression of endogenous Tgdf1 in a somatic cell.

In some embodiments, an isolated stable reprogrammed cell has at least a 5-fold higher expression of endogenous Tert as compared to the level of expression of endogenous Tert in a somatic cell, for example, at least a 7-fold higher expression, or between a 7-fold and 20-fold higher expression of endogenous Tert as compared to the level of expression of endogenous Tert in a somatic cell.

In some embodiments, an isolated stable reprogrammed cell has at least a 100-fold higher expression of endogenous Sox2 as compared to the level of expression of endogenous Sox2 in a somatic cell, for example, at least a 500-fold higher expression, or between about a 500-fold and 100,000-fold higher expression, or between about a 500-fold and 10,000-fold higher expression of endogenous Sox2 as compared to the level of expression of endogenous Sox2 in a somatic cell.

In some embodiments, an isolated stable reprogrammed cell has a lower expression of Krt10 or Thy1 or Krt10 and Thy1 by a statistically significant amount relative to the level of expression of the isogenic cell from which the reprogrammed cell was derived.

In some embodiments, an isolated stable reprogrammed cell has a n increased CpG methylation of Oct4 or Nanog or Oct4 and Nanog a statistically significant amount relative to the level of CpG methylation of Oct4 or Nanog or Oct4 and Nanog an induced pluripotent stem (iPS), and/or a decreased CpG methylation of Oct4 or Nanog or Oct4 and Nanog a statistically significant amount relative to the level of CpG methylation of Oct4 or Nanog or Oct4 and Nanog an the somatic cell, such as an isogenic cell from which the reprogrammed cell was derived.

In some embodiments, an isolated stable reprogrammed cell has an increased rate of proliferation by a statistically significant amount relative to the level of expression of an induced pluripotent stem (iPS) cell.

In some embodiments, an isolated stable reprogrammed cell has an increased expression of Xist in female piPS lines by a statistically significant amount to the level of expression of the somatic cell from which the reprogrammed cell was derived, for example, at least a 5-fold higher expression of Xist, or between about a 5-fold and about a 50-fold higher expression of Xist, or between about a 5-fold and about a 20-fold higher expression of Xist in female lines as compared to the level of expression of endogenous Xist in a female induced pluripotent stem (iPS) cell.

In some embodiments, an isolated stable reprogrammed cell has is not an induced pluripotent stem (iPS) cell, where the iPS cell can not be reprogrammed any further, whereas the stable reprogrammed cell (e.g. piPSC) can be further reprogrammed to a more undifferentiated state.

In some embodiments, an isolated stable reprogrammed cell has not undergone complete remodeling of the epigenome. In some embodiments, an isolated stable reprogrammed cell has undergone a significantly less amount of remodeling of the epigenome as compared to an induced pluripotent stem (iPS) cell, for example, a reprogrammed cell has undergone at least about 10%, or at least about 20%, or at least about 30%, or at least about 40%, or at least about 50%, or at least about 60%, or at least about 70%, or more than 70% less remodeling of the epigenome as compared to an induced pluripotent stem (iPS) cell female induced pluripotent stem (iPS) cell. Stated another way, where an iPSC has undergone global or 100% remodeling of the eipgenome, a stable reprogrammed cell as disclosed herein, e.g. a piPSC, the epigenome has undergone less than 100% remodeling, for example less than 90%, or less than 80%, or less than about 70%, or less than about 60%, or less than about 50%, or less than about 40%, or less than about 30%, or less than about 20%, or less than about 10% of the epigenome is remodeled.

In some embodiments, an isolated stable reprogrammed cell can be further reprogrammed into an induced pluripotent stem (iPS) cell. In some embodiments, an isolated stable reprogrammed cell is a multipotent cell.

In some embodiments, an isolated stable reprogrammed cell has at least a 10-fold lower, or at least about 15-fold lower expression of Dnmt3b relative to the level of expression of an induced pluripotent stem (iPS) cell.

In some embodiments, an isolated stable reprogrammed cell has is capable of multilineage differentiation, for example, along neuronal lineages, mesoderm and ectoderm lineages. In some embodiments, the efficiency of differentiation along different lineages differs between clones of piPSC as disclosed herein in the Drawings. In some embodiments, some reprogrammed cells differentiate along neuronal lineages with greater efficiency than other reprogrammed cells. In some embodiments, an isolated stable reprogrammed cell can differentiate along neuronal lineages, for example, a reprogrammed cell can differentiate along neuronal lineages at a significantly higher efficiency than that of an induced pluripotent stem (iPS) cell.

Another aspect of the present invention relates to a method for producing a reprogrammed cell of claim 1, the method comprising; (i) contacting a isogenic cell with at least one of either an exogenous transcription factor or at least one of an agent which replaces an exogenous transcription factor, wherein the exogenous transcription factor is selected from the group consisting of; Sox2, Oct-4, Klf-4, c-Myc, lin-28 and Nanog; and (ii) isolating a reprogrammed cell as disclosed herein. In some embodiments, an exogenous transcription factor is a nucleic acid encoding at least one of the transcription factors selected from the group consisting of: Sox2, Oct-4, Klf-4, c-Myc, lin-28 and Nanog.

In some embodiments, an exogenous transcription factor is at least one compound selected from the group consisting of; (a) a TGF-β Receptor Type I inhibitor, wherein the TGF-β Receptor Type I inhibitor substitutes for exogenously Sox2 transcription factor, and wherein exogenous Sox2 transcription factor is not present (e.g. RepSox (E-616452), or SB43142, or E-616451), (b) an inhibitor of Src signaling pathway, wherein the inhibitor of Src signaling pathway substitutes for exogenously Sox2 transcription factor, and wherein exogenous Sox2 transcription factor is not present; (e.g. EI-275); (c) an agonist of the Mek/Erk signaling pathway, wherein agonist of the Mek/Erk signaling pathway substitutes for exogenously Klf-4 transcription factor, and wherein exogenous Klf-4 transcription factor is not present; (e.g. PGJ2); (d) an inhibitor of Ca2+/calmodulin, wherein the inhibitor of Ca2+/calmodulin signaling pathway substitutes for exogenously Klf-4 transcription factor, and wherein exogenous Klf-4 transcription factor is not present; (e.g. HBDA); (e) an inhibitor of EGF signaling, wherein the inhibitor of EGF signaling pathway substitutes for exogenously Klf-4 transcription factor, and wherein exogenous Klf-4 transcription factor is not present; (e.g. HBDA); (f) an agonist of ATP-dependent potassium channels, wherein the agonist of ATP-dependent potassium channels substitutes for exogenously Oct-4 transcription factor, and wherein exogenous Oct-4 transcription factor is not present; (e.g. Simomenine); (g) a sodium channel inhibitor, wherein the inhibitor of sodium channels substitutes for exogenously Oct-4 transcription factor, and wherein exogenous Oct-4 transcription factor is not present; (e.g. Simomenine) (h) an MAPK agonist, wherein the MAPK agonist substitutes for exogenously Oct-4 transcription factor, and wherein exogenous Oct-4 transcription factor is not present; (e.g. Ropivocaine or Bupivicanine).

Another aspect of the present invention relates to an isolated heterogeneous population of stable reprogrammed cells comprising at least two different stable reprogrammed cell populations.

Another aspect of the present invention relates to a heterogeneous population of stable reprogrammed cells and iPSCs.

Another aspect of the present invention relates to the use of a stable reprogrammed cell for differentiating into a isogenic cell of endoderm lineage, mesoderm lineage and ectodermal lineage, wherein the reprogrammed cell does not become an induced pluripotent stem (iPS) cell prior to differentiating into a cell of endoderm lineage, mesoderm lineage and ectodermal lineage.

In some embodiments, an isolated stable reprogrammed cell can be used for producing an induced pluripotent stem (iPS) cell. In some embodiments, an isolated stable reprogrammed cell can be cultured in the absence of LIF, or in the presence of an exogenous transcription factor or an agent which replaces an exogenous transcription factor, e.g., RepSox (E-616452), or other agent, such as an agent which replaces an exogenous transcription factor, wherein the exogenous transcription factor is selected from the group consisting of; Sox2, Oct-4, Klf-4, c-Myc, lin-28 and Nanog.

In some embodiments, an isolated stable reprogrammed cell as disclosed herein can be used for generating a disease model, e.g. a disease model in vitro or in vivo model, e.g., a disease animal model, e.g., a rodent animal model.

Another aspect of the present invention relates to a differentiated cell derived from inducing the differentiation of a stable reprogrammed cell, e.g., piPSC as disclosed herein.

Another aspect of the present invention relates to a method for evaluating the toxicity of an agent comprising contacting a reprogrammed cell with a stable reprogrammed cell as disclosed herein with an agent and evaluating the effect of the agent on at least one of the following characteristics of the reprogrammed cell; multilineage differentiation capacity into all 3 germline cell layers, viability, propagation for at least 20 passages.

Another aspect of the present invention relates to a method for stem cell therapy comprising; (i) isolating and collecting a isogenic cell, e.g., a somatic cell from a subject; (ii) reprogramming the isogenic cell to a reprogrammed cell as disclosed herein, (iii) inducing differentiation of the reprogrammed cell; and (iv) transplanting the differentiated cell from step (iii) into the subject. In some embodiments, the subject is an animal model, and in some embodiments, the subject is a human. In some embodiments, the reprogrammed cell is a mammalian cell, e.g., a human cell.

Another aspect of the present invention relates to a method to isolate a stable reprogrammed cell as disclosed herein from a population of cells comprising induced pluripotent stem cells (iPSCs) and somatic cells, the method comprising; (i) positively selecting for cells with a statistically significant high level of expression at least one of Tdgf1, Tert, Sox2, Pou5f1; (ii) selecting the cells obtained in step (i) for cells with a significantly low level of expression of at least one of Dnmt3b, Dnmt3a, Rex1, Left2, or Nanog, wherein the cells selected in step (ii) are an isolated reprogrammed cell.

Another aspect of the present invention relates to a method to isolate a stable reprogrammed cell from a population of cells comprising induced pluripotent stem cells (iPSCs), the method comprising positively selecting for cells with a significantly low level of expression of at least one of Dnmt3b, Dnmt3a, Rex1, Left2, or Nanog.

Another aspect of the present invention relates to a method to isolate a stable reprogrammed cell as disclosed herein from a population of cells comprising induced pluripotent stem cells (iPSCs), the method comprising negatively selecting for cells with a significantly high level of expression of at least one of Dnmt3b, Dnmt3a, Rex1, Left2, or Nanog, wherein the cells with a significantly high level of expression of at least one of Dnmt3b, Dnmt3a, Rex1, Left2, or Nanog are iPSC and are discarded, and wherein the cells which do not express significantly high level of expression of at least one of Dnmt3b, Dnmt3a, Rex1, Left2, or Nanog are collected and are stable reprogrammed cells (e.g., piPSC) as disclosed herein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1B show retroviral infections with the pMXs vector (Takahashi et al., 2007a). MEFs were infected with two to three pools of viral supernatant during a 72 hr period. FIG. 1A shows brightfield morphology of the transfected MEF at ×4 magnification, and FIG. 1B shows a higher magnification of ×10. GPF expression from expression from the Oct4:GFP vector was detected (data not shown).

FIGS. 2A-2B show how exemplary piPS cell lines generated. FIG. 2A shows a table of 30 exemplary piPS cell lines generated. Of the thirty, ten lines were selected randomly for further characterization. Four of the ten lines were derived using the HDAC inhibitor valproic acid (VPA). FIG. 2B shows a schematic of the intermediate stable reprogrammed cells which can be further reprogrammed to fully and terminally reprogrammed iPS cells. On the right shows a panel of 9 different stable reprogrammed cell lines which retain pluripotenty and pluripotent stem cells marker expression for at least 20 passages or more.

FIGS. 3A-3B shows bright field image of the morphology of exemplary piPS cell lines generated. FIG. 3A shows exemplary images of piPSC colonies after just reprogrammed to piPS cells. FIG. 3B shows bright field image of the morphology of exemplary piPS cell lines cultured and propagated in vitro for more than 20 passages. To examine if piPSC cell lines exhibited a stable phenotype that could be cultured and propagated in vitro piPC were cultured under standard mES growth conditions for greater than twenty passages on mitotically inactive mouse embryonic fibroblast feeder layer. Over the long-term culture, piPS cell lines continued to grown in colonies that maintained a similar morphology as observed with the starting population of cells.

FIGS. 4A-4B show exemplary bright field images of partially reprogrammed piPSC lines. FIG. 4A shows image obtained from piPSC lines cultured on gelatin. FIG. 4B shows images obtained from piPSC lines cultured on mitotically inactive MEF feeder layer.

FIGS. 5A-5C show expression of Nanog, Oct4, Sox2, Klf4, cMyc in exemplary piPS cell lines as compared to MEFs and iPSCs. FIG. 5A shows total mRNA of Nanog, Oct4, Sox2, expressed in exemplary piPS cell lines as compared to MEFs and iPSCs. FIG. 5B shows levels of virally expressed Klf4, Sox2, Oct4 and cMyc. FIG. 5C shows expression of GAPDH as an endogenous loading control.

FIG. 6 shows the expression of piPSC heterogeneously upregulate differentiation markers upon differentiation spontaneous differentiation in low serum.

FIG. 7 shows the sequence of the Oct4 promoter and locations of CpG Islands and primers for Bisulfite Sequencing of the oct4 promoter.

FIGS. 8A-8C show piPS cell lines proliferate at a faster rate, and have a faster doubling time as compared to iPSCs and MEFs. piPS, iPS and mES cells were seeded onto identical plates in triplicate. To determine doubling time the cells were counted approximately every 24 hours over a period of 96 hours. Cells were harvested by trypsinization and counted. FIG. 8A shows results from a cell proliferation assay, showing the increase in cell number over a 100 hour time period. FIG. 8B is a histogram of the doubling time of mES cells, iPSC cells and piPSC. FIG. 8C shows the doubling time measured in hours of a variety of exemplary piPS cells as compared to iPSC and mESCs (mouse embryonic stem cells). Populations of piPSC had a doubling rate about twice the rate than iPSC or mESC, where piPSC doubled about every 10 hours, whereas iPSC and mESCs doubled about every 20 hours.

FIG. 9 shows a table demonstrating that failure of piPSCs to fully reprogram to an iPSC is not due to deficiency of a single reprogramming factor. Stable piPS cell lines were infected with three rounds of viral supernatant diluted 1:8 in MEF media in a 48-hour period on gelatin or feeder MEFs. Four-teen days after the last viral transduction lines were examined for the appearance of Oct4::GFP positive colonies.

FIGS. 10A-10B show that piPS cell lines can be further reprogrammed to iPSC using Repsox and 2i. A subset of piPS cell lines were treated with RepSox (E-616452) (25 μM), AZA (500 μM), or both for 48 hours. For 2i treatment, CHIR99021 (Stemgent) was used at 3 μM and PD0325901 (Stemgent) was used at 1 μM. Oct4::GFP+ colonies were scored 12 days after the beginning of chemical treatment. Treatments were performed in mES media containing FBS unless otherwise noted. FIG. 10A shows on MEFS, the number of piPSCs from each piPSC line which expressed GFP demonstrating further reprogramming to iPSCs. FIG. 10B shows the number of piPSCs from each piPSC line which expressed GFP demonstrating further reprogramming to iPSCs on 0.1% gelatin.

FIG. 11 shows piPSC spontaneously form embryoid bodies (EB) upon removal of LIF.

FIG. 12 shows histology images of piPSC which form teratomas containing tissue from the three embryonic germ layers upon injection into nude mice. Immunohistochemical staining demonstrates different piPS lines differentiate along all three lineages; endoderm, mesoderm and ectoderm lineages.

FIGS. 13A-13B shows a subset of piPSC cell lines exhibit preferential bias towards ectodermal differentiation. FIG. 13A shows double immunostaining with Tuj1 and HB9 to demonstrate that piPS cells can be directed to differentiate into post-mitotic motor neurons. piPS cell cultures were fixed with PFA and stained with primary antibodies against TUJ1 (Sigma, T2200) and HB9 (Developmental Studies Hybridoma Bank, 81.5C10), and visualized by staining with secondary antibodies conjugated to Alexa Fluor 488 and Alexa Fluor 546 (Invitrogen). FIG. 13B shows a table of the efficiency of piPS cell differentiation into motor neurons. For counting HB9+ cells, embryoid bodies were sectioned as above and stained with the TUJ1 and HB9 antibodies along with the Alexa Fluor 488 and Alexa Fluor 546 secondary antibodies. Cultures were counterstained with Hoechst 33342 and HB9+ and total nuclei were counted. Numbers were derived from at least 3 different embryoid bodies per cell line.

FIG. 14A-14F show piPSC exhibit differences in differentiation potential. FIG. 14A shows immunostaining with Tuj1 on day 8 in piPSC line B. FIG. 14B shows positive immunostaining for Sox17+ cells on day 8 in piPSC I. FIG. 14C shows positive immunostaining for Alpha-actinin+ cells on day 12 in piPSC C. FIG. 14D is a histogram showing Tuj expression in piPSC, iPSC and mESC lines after 8 days after directed differentiation. FIG. 14E is a histogram showing Sox17 expression in piPSC, iPSC and mESC after eight days directed differentiation. FIG. 14F shows the percent of contractile EBs in piPSC, iPSC and mESC 12 days after directed differentiation.

FIG. 15A-15B show progenitor state of piPS neurospheres. FIG. 15A shows immunostaining with nestin (a marker for neuronal stem cells and neuronal progenitors) which demonstrate that some piPS lineages differentiate along neuronal lineages at a higher efficiency as other cell lines. FIG. 15B shows immunostaining with Tuj1 (marker for immature neurons), demonstrating that some piPS lineages preferentially differentiate along neuronal lineages at a higher efficiency as compared to other cell lines.

FIG. 16 is a schematic outlining the methods for determining cell characteristics, cell cycle and differentiation properties of different piPS lineages.

FIG. 17 shows Tuj1 immunostating of different piPS lineages demonstrating differentiation along neuronal lineages.

FIG. 18 shows neurogenic potential of iPS Cvs piPSC. piPS lines 22 and 2 have a greater efficiency of differentiating along neurogenic lines and a higher neurogenic potential that iPS cells.

FIG. 19 shows neurogenic potential of iPS Cvs piPSC. Some exemplary piPS lines have a greater efficiency of differentiating along neurogenic lines and a higher neurogenic potential that iPS cells.

FIG. 20A-20C show a subset of piPS exhibit a preferential bias towards meosoderm differentiation. FIG. 20A shows bright field image of piPS which exhibit a preferential bias towards mesoderm differentiation, which expressed alpha actin in a cardiomyocyte type morphology (data not shown). FIGS. 20B and 20C shows histograms of the percentage of piPSC which have differentiated into beating cardiomyocytes, showing the efficiency of piPS cell differentiation into beating cardiac myocytes.

FIG. 21 is a schematic demonstrating a method for differentiating piPS to Endodermal differentiation.

FIG. 22 is a table of the expression levels of a variety of different markers expressed in piPS cell lineages as compared to iPSC and mES and MEFs, as well as their differentiation into ectoderm, mesoderm and endodermal differentiation lineages in vitro and in vivo.

FIGS. 23A-23D show the morphology of exemplary piPSCs as compared to iPSC morphology. Bright field images of iPS and piPS cell colonies grown on mouse embryonic fibroblasts. FIG. 23A show the morphology of a typical iPSC colonies, which form smooth colonies with refractory edges. FIGS. 23B, 23C and 23D show exemplary piPS cell colonies, which exhibit a more granulated morphology punctuated by the borders of individual cells. In addition, the edges of individual piPS colonies can appear rather jagged or rough and typically are not refractory.

FIG. 24 shows quantitative expression of pluripotency genes Dnmt3b, Lefty2, Nanog, Tdgf1, Tert, Sox2, Xist, Pau5f1, Zfp42 in piPS cells compared to iPSC cells and somatic cells, e.g., MEFs. Gene expression was measured using real-time RT-PCR. Expression of all genes was normalized to 18s rRNA and calibrated to expression in mouse embryonic fibroblasts (MEFs).

FIG. 25A-25C show piPSC cells are molecularly distinct from iPSC and MEFs, and piPSC cell lines display heterogeneous staining for SSEA1 and AP between lines. FIG. 25A shows piPSC line C exhibit small granular AP-, SSEA-cells. FIG. 25B shows piPSC line A forms compact colonies that diffusely stain for AP and are absent for strongly SSEA1. FIG. 25C shows piPSC line I forms compact colonies that stains strongly for SSEA1 and AP.

FIG. 26A-26J shows piPSC cells are molecularly distinct from iPSC and MEFs. FIG. 26A shows a heat dendrogram diagram of the results of a gene expression analysis of piPSC lines, iPSC, mESC and MEF lines. Expression levels were normalized to 18s rRNA and calibrated to expression levels in MEFs. The dendrogram was generated by complete linkage hierarchical clustering using Pearson correlation on all measured genes. Red, black and green represent higher, identical, and lower expression levels, respectively. FIGS. 26B-26J show taqman Real-time RT-PCR using primer and probe sets specific for genes associated with pluripotency, and self-renewal. FIGS. 26B and 26C show piPS cells do not express Col1a1, Col2a1 as compared to MEFs. FIG. 26D show piPS cells express lower levels of Nanog as compared to mES cells or iPS cells. FIG. 26E shows piPS cell lines express significantly lower levels of cripto as compared to mES cells or iPS cells. FIG. 26F shows piPS cell lines express significantly higher levels of Lefty2 as compared to MEF cells, but significantly lower levels as compared to mES cells or iPS cells. FIG. 26G shows piPS cell lines express significantly higher levels of Tert as compared to MEF cells, but not significantly different levels of Tert as compared to mES cells or iPS cells. FIG. 26H shows piPS cell lines express significantly higher levels of Rex1 as compared to MEFS. FIG. 26I shows piPS cell lines express at least 2-fold higher levels of Lin28 as compared to MEFs, and about at least a 2-fold or about a 10-fold lower expression as compared to mES cells or iPS cells. FIG. 26J shows piPS cell lines express significantly lower levels of FGF4 as compared to mES cells or iPS cells, and a significantly higher level of expression as compared to MEFs.

FIGS. 27A-27C shows that piPSCs are epigenetically distinct from iPSC and MEFs. FIG. 27A shows de novo methyltransferase expression (Dnmt3b) in piPSC, mESC, and iPSC, and demonstrates that piPSC lines express lower de novo methyltransferase than mESC but higher than MEFs (p>0.0001). FIG. 27B shows that piPSCs have intermediate levels of DNA CpG methylation on the promoter regions of Oct4, and that piPS cells have significantly more methylation on the Oct4 promoter region as compared to iPS cells, and significantly less methylation on the Oct4 promoter region as compared to MEFs. FIG. 27C shows that piPSCs have intermediate levels of DNA CpG methylation on the promoter regions of Nanog and that piPS cells have significantly more methylation on the Nanog promoter region as compared to iPS cells, and significantly less methylation on the Nanog promoter region as compared to MEFs. Unfilled circles indicate unmethylated and filled circles indicate methylated CpG dinucleotides.

FIGS. 28A-28C shows piPSCs respond differentially to reprogramming chemicals. FIG. 28A shows RepSox could be used to further reprogram intermediate piPSC lines to fully reprogrammed iPSC cells. 10× bright field image merged with Oct4::GFP. The concentration of DMSO equals 0.2%. FIG. 28B shows a subset of piPSC lines reprogram when exposed to 25 μM RepSox and 2i. piPSC lines were exposed with chemical for 48 hours and scored 12 days later for the presence of Oct4::GFP positive colonies. FIG. 28C shows piPSC lines D, F, G, and E were treated with 25 μM RepSox for 48 hours, and at 96 hours RNA was harvested and the expression of Nanog was examined via RT-PCR.

FIGS. 29A-29C show reprogramming response of stable intermediate piPS cells correlates with histone modification associated with the Nanog promoter. FIG. 29A shows DNA methylation of Nanog does not differ significantly (p<0.0001) between responsive and non-responsive piPS cell lines, whereas methylation of fully reprogrammed iPS cells has a significantly lower level of DNA methylation of Nanog, and MEFs have significantly higher level of Nanog DNA methylation as compared to piPS cells. FIG. 29B shows chromatin state of Nanog proximal promoter. Genomic DNA as-associated with modified histones was immunoprecipitated using either an anti-H3K4me2 or anti-H3K27me2 antibody. Fold-enrichment was calculated as the relative enrichment from a non-specific control. FIG. 29C shows Oct4 promoter exhibits higher levels of H3K27 dimethylation as compared to H3K4 dimethylation. Genomic DNA associated with modified histones was immunoprecpitated using either an anti-H3K4me2 or anti-H3K27me2 antibody. Fold-enrichment was calculated as the relative enrichment from a non-specific control.

FIG. 30 is a schematic diagram illustrating that the piPS cells can be differentiated into cells of all three germ lines in vitro by culturing under different differentiation conditions, to generate cardiomyocytes, neuroectodermal precursors and definitive endoderm cells. The bottom panel of histograms illustrates some piPS cell lines have a higher efficiency to differentiate into specific cell lineages.

FIG. 31 shows piPSC maintain active expression of all transgenic reprogramming factors.

FIG. 32A-32B show piPSC cells do not reprogram further with reprogramming factors. FIG. 32A shows reprogramming efficiency (by % GFP expression) on viral transduction on MEFs with a variety of reprogramming factors. FIG. 32B shows reprogramming efficiency (by % GFP expression) on viral transduction on gelatin with a variety of reprogramming factors.

FIG. 33A-33C shows linage specific genes are heterogeneously upregulated in piPSC lines. FIG. 33A shows selected genes with mesodermal lineages. FIG. 33B shows expression of ectodermal linage genes. FIG. 33C shows expression of endodermal lineage genes.

FIG. 34 shows a table of the pearson's coefficients between piPSC, iPSC, mESC and MEF cells.

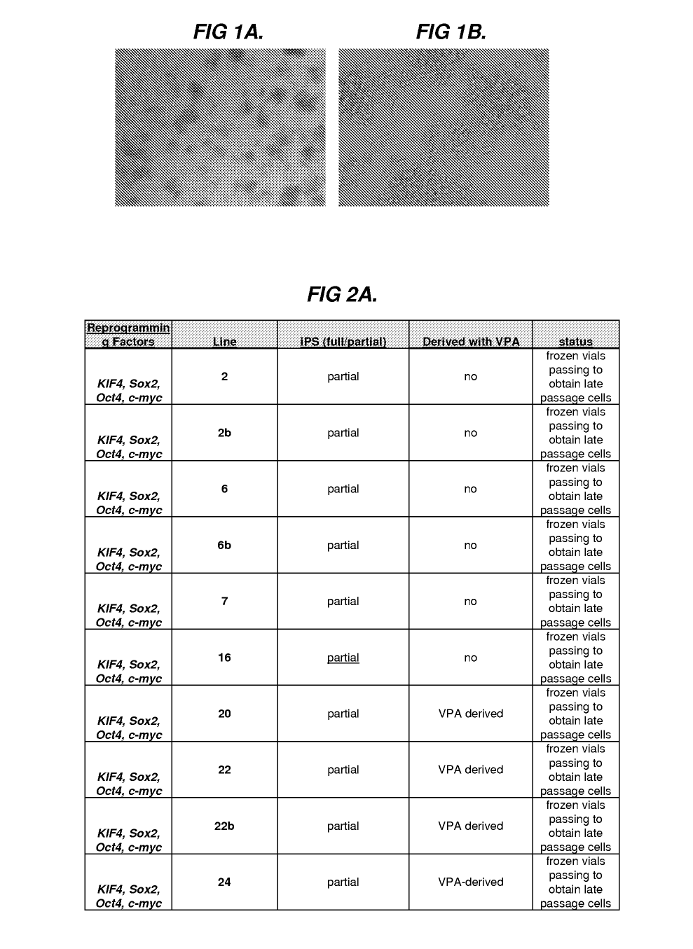

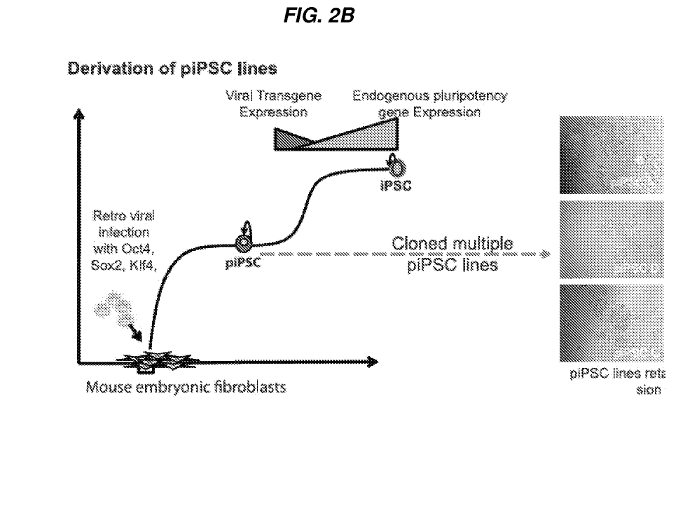

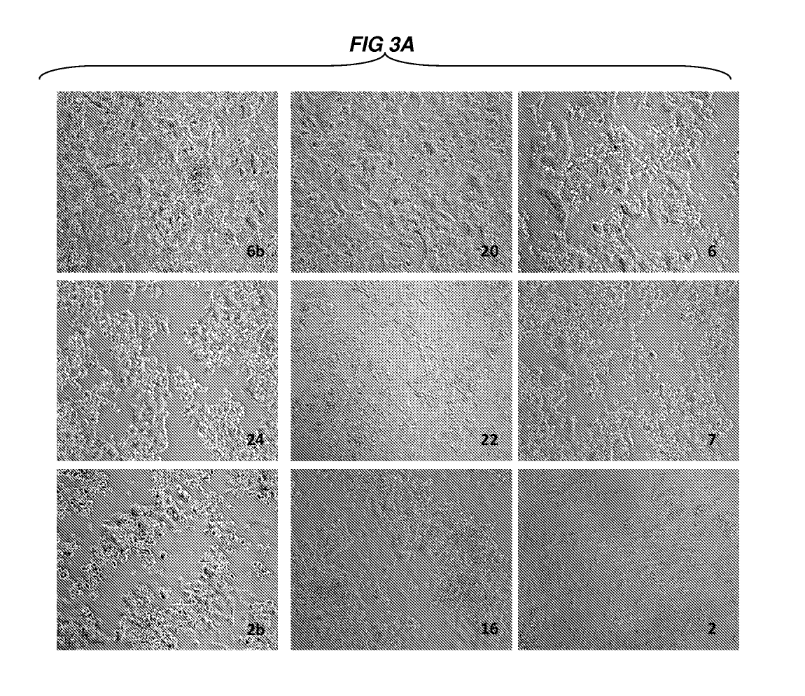

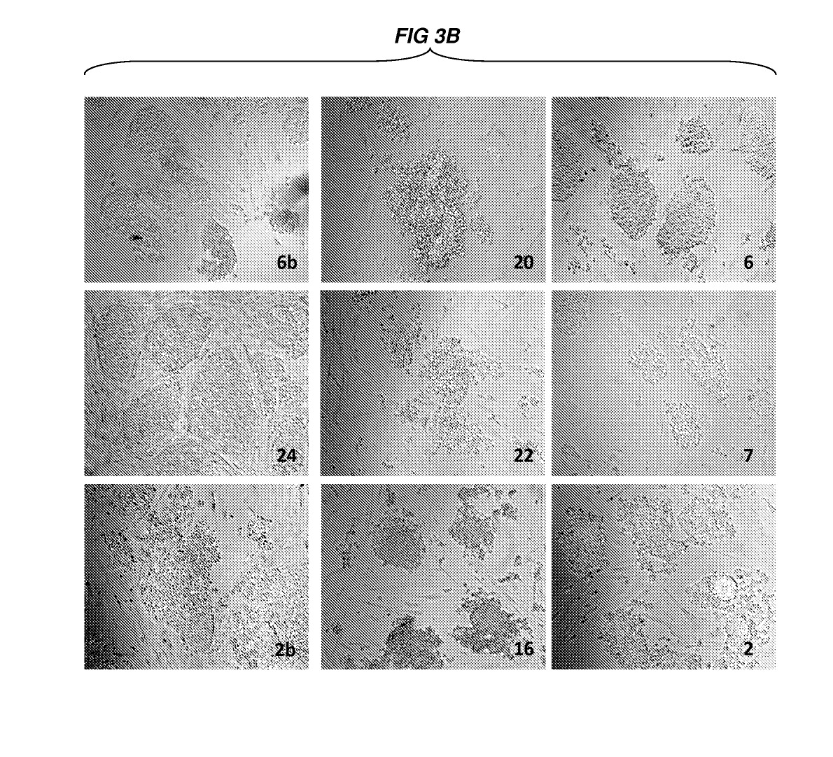

### DETAILED DESCRIPTION OF THE INVENTION

The present invention relates generally to a pluripotent stable reprogrammed cell, and compositions comprising a stable reprogrammed cell, where the cell is reprogrammed from a somatic cell, and wherein the cell has not undergone complete reprogramming to an induced pluripotent cells (iPSC). In some embodiments, unlike an iPSC, which cannot be further reprogrammed, a stable reprogrammed cell as disclosed herein can be further reprogrammed to become an iPSC. Other aspects of the present invention relate to methods to isolate a stable reprogrammed cell, herein referred to a partial induced pluripotent stem cell or piPS cell.

The advantages of the stable reprogrammed cell as disclosed herein include preventing global remodeling of the epigenome of the cell which occurs with complete reprogramming or in the production of iPS cells. For example, viral transduction of three transcription factors reprograms adult somatic cells to induced pluripotent cells that possess many of the known characteristics of embryonic stem cells including global remodeling of the epigenome. Complete remodeling of the epigenome may be disadvantageous for studying disease phenotypes that are encoded in the epigenetic memory of a cell. In this respect, partially reprogrammed iPS cells disclosed herein are advantageous in that they more faithfully retain the epigenetic memory of a diseased state when differentiated to a clinically relevant cell type. Differentiation of epigenetic disease relevant cell types may serve as a valuable resource for disease modeling and therapeutic screening. To this end, the piPS can be used to generate disease relevant cell types obtained from a partially pluripotent state which will maintain a stronger imprinting of diseased states than those obtained from bona fide induced pluripotent cells.

In some embodiments, a stable reprogrammed cells as disclosed herein, can be produced by retroviral infections as previously described with the pMXs vector (Takahashi et al., 2007a). For example, a somatic cell, e.g., a fibroblast, e.g., a mouse embryonic fibroblast (MEF) can be infected with two to three pools of viral supernatant during a 72 hr period. The first day that viral supernatant was added was termed “day 1 post-infection.” For quantification, Oct4::GFP negative colonies were isolated by manual selection approximately 30 days post transduction. Oct4::GFP negative colonies were subsequently sub cloned an additional two times to insure clonality. Following sub cloning, piPS cell lines were cultured and passaged following standard mESC protocols. All animal research was performed under the oversight of the Office of Animal Resources at Harvard University.

The stable reprogrammed cells, herein also referred to a “partially induced pluripotent stem cells” or “piPS” cells are pluripotent and can form immature, small teratomas in vivo. For example, when injected into an immune-compromised mouse, piPS cell lines form teratomas. Teratomas formed from piPS cell contain tissues of all three embryonic germ-layers. In some embodiments, piPS teratomas contain an abundance of neuronal tissues. In some embodiments, as apposed to tetromas formed from in vivo implantation of iPSC, the majority of teratomas formed by piPS cell lines lack well defined encapsulations. In contrast, bona fide iPS cell lines yield well encapsulated teratomas containing abundant tissue from all three germ-layers. This demonstrates that teratomas obtained from piPS cells are less mature than teratomas obtained from iPSC. This further demonstrates that piPS are in a less pluripotent state than iPS cells but a state that is nonetheless capable of multilineage differentiation.

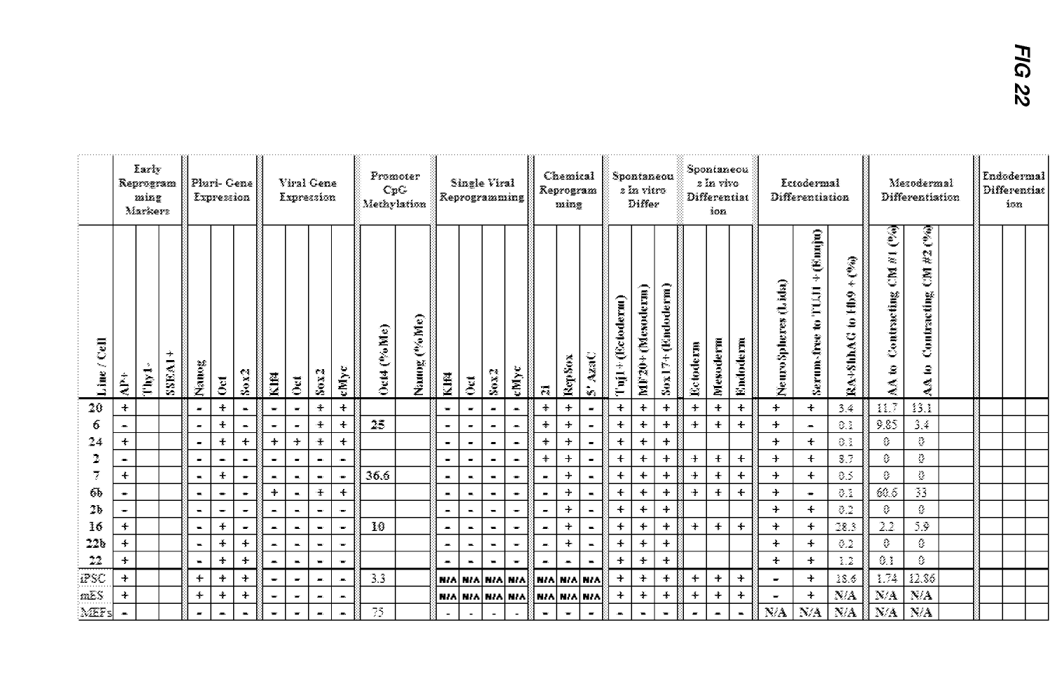

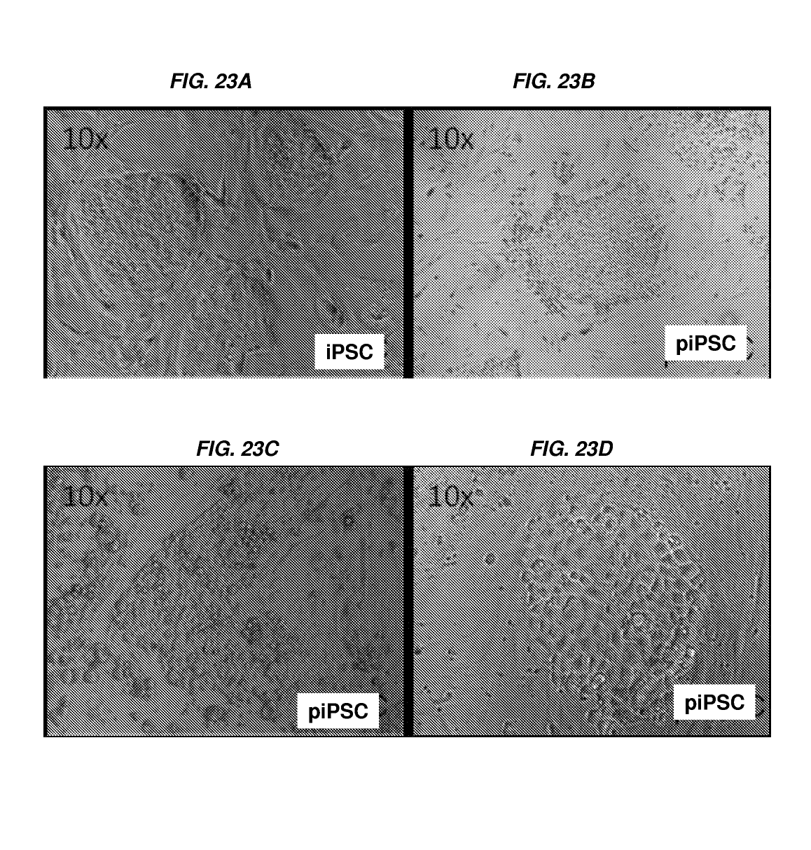

In some embodiments, the stable reprogrammed cells, e.g., piPS cells have a different and distinct morphology to iPSCs in that piPSC or stable reprogrammed cells form a flat sheet of cells, and defined boundaries. In particular, in some embodiments, piPSC, like iPS/ES cells form compact colonies when cultured on fibroblast feeders or gelatin. However, piPS colonies have several morphological distinctions. In contrast to iPS/ES cells, piPS colonies are typically composed of a flat sheet of cells. Cells within a piPS colony typically have defined boundaries allowing easy visualization of individual cells within a colony. This is in contrast to iPS/ES cell colonies which typically exhibit a smooth morphology where the boundaries between individual cells in a colony are poorly defined. The edges of piPS cell colonies are also distinct. iPS/ES cell colonies typically have smooth, rounded surfaces that are refractory or shiny when visualized via bright field microscopy. In contrast, the edges of piPS cell colonies are generally not smooth, rounded or refractory as shown in FIG. 23A-23D.

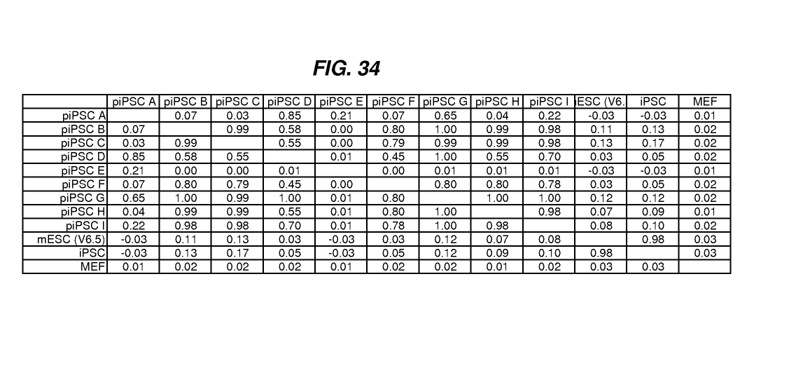

Further, stable reprogrammed cells e.g., piPSC can be distinguished from iPSC based on expression of markers, or cell-surface markers. In some embodiments, one of ordinary skill in the art can isolate piPS cells from iPS cells, for example, in a two-step positive and negative selection scheme. In some embodiments, a first step would isolate both iPSC and piPSC by selecting cells that express a marker that is ubiquitously expressed in both iPSC/ESC and piPSC. In some embodiments, an ideal candidate marker might encompass expression and activity of one or more of the following: mTert, Dnmt3b, Dnmt3a, Rex1, Left2, or Tdgf1. In some embodiments, a second negative selection methods would select cells that do not express or contain the activity of marker only expressed in iPSC/ESC. An ideal marker would be Nanog. In some embodiments, the other markers for this second selection can be, for any one or a combination of Dnmt3b, Lefty2 or Nanog, as shown in FIG. 34.

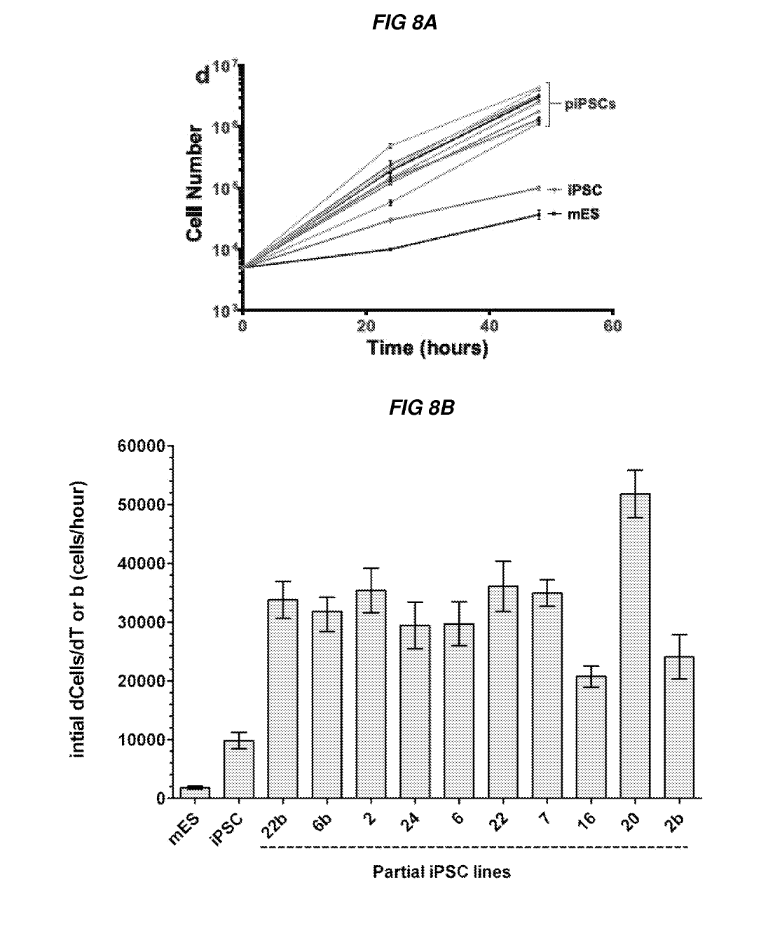

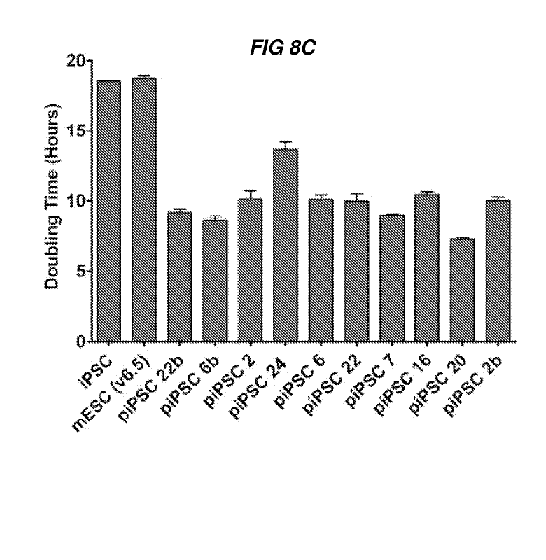

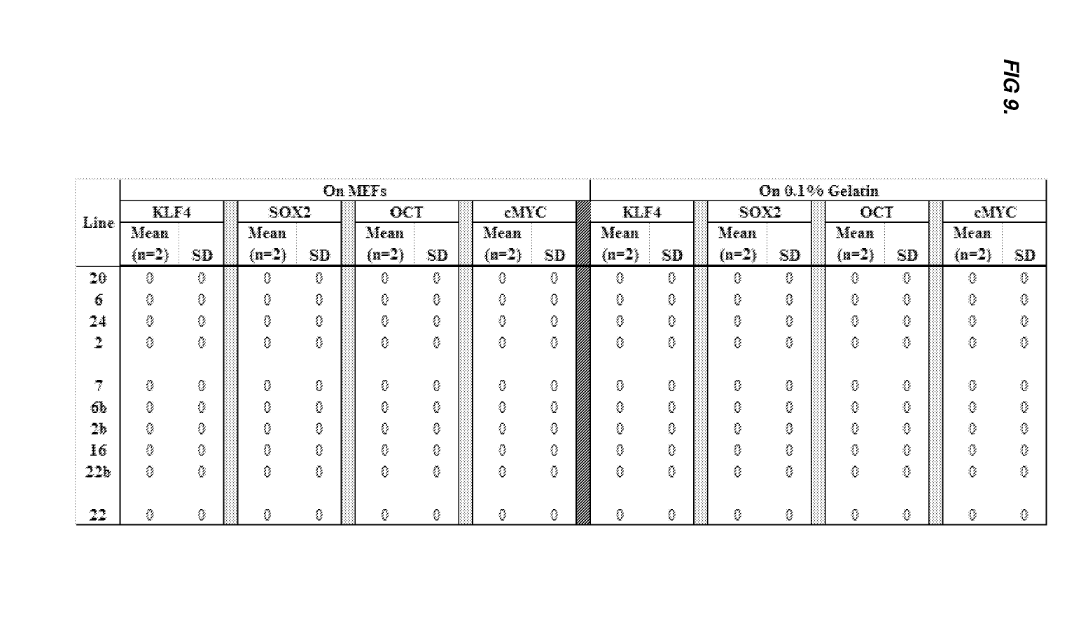

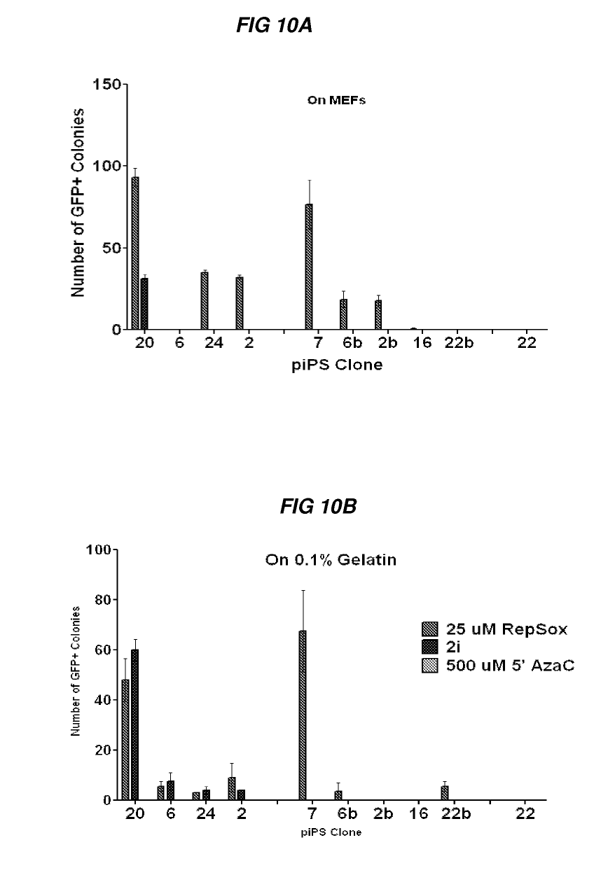

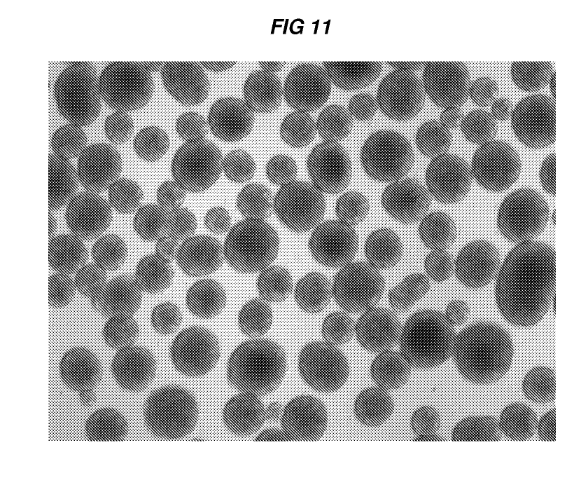

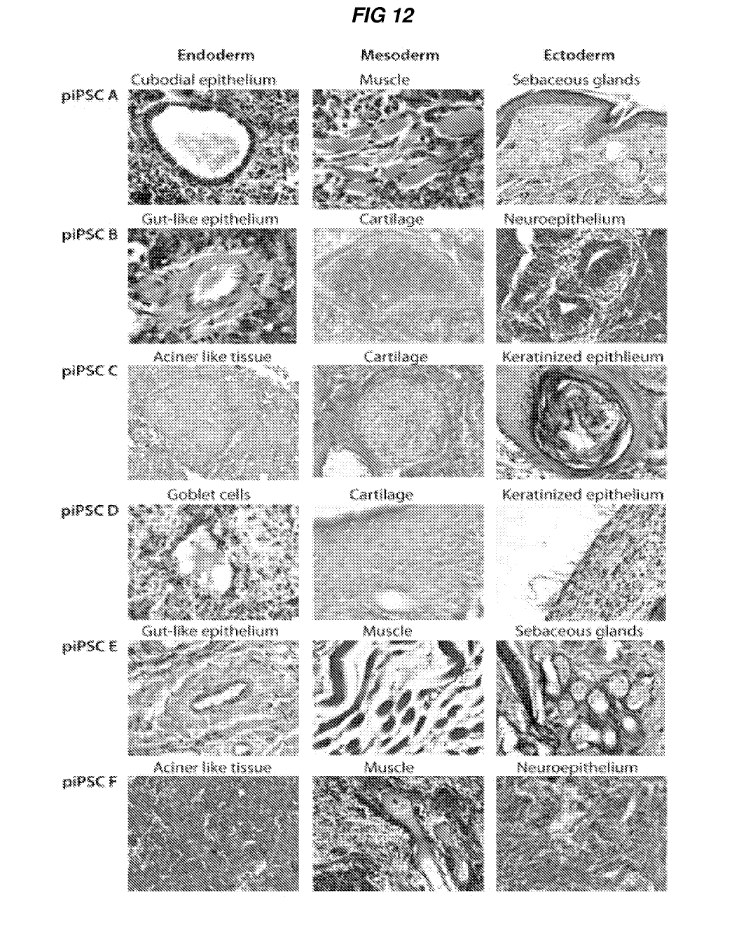

Further, piPSC can be distinguished from iPSC or embryonic SC based on doubling times. As shown in FIG. 8C, piPCS have at least a 1.5-fold faster doubling time as compared to iPSC, or at least a 2.0-fold faster doubling time as compared to iPSC. Doubling times can be assessed using assays well known by those of ordinary skill in the art, for example, clonogenic assays and can be easily calculated for example at the world-wide web site “doubling-time.com/compute.php”.

Interestingly, when the inventors compared of the proliferation and differentiation potential of 10 lines of piPS to the bona fide iPS and ESC, the inventors determined that both iPS and ESC failed to form neurospheres, whereas in contrast, all 10 piPS lines were able to maintain their self-renewing capacity for 3 passages (˜3 weeks).

In some embodiments, a population of stable reprogrammed cells is a clonally isolated population of stable reprogrammed cells which can differentiate into a specific linage of interest.

In some embodiments, a population of stable reprogrammed cells is a heterogeneous population of stable reprogrammed cells which can differentiate into a variety of different lineages of interest. In some embodiments, a heterogeneous population of stable reprogrammed cells comprises a mixture of stably partially reprogrammed cells with distinct characteristics, where different cells can preferentially differentiate into a specific cell lineage, e.g., including but not limited to, neurons and muscle cells.

In some embodiments, a population of stable reprogrammed cells as disclosed herein can be used as a standard, e.g., a calibration tool, in an assessment, and/or assays to identify cell lines, e.g., iPS cell lines which comprise, at least in part, some reprogrammed cells, e.g., piPSC which are incomplete or not fully reprogrammed cells.

In some embodiments, a population of stable reprogrammed cells as disclosed herein can be used in kits, for example, as a standard, e.g., a calibration tool, in an assessment, and/or assays to identify cell lines, e.g., iPS cell lines which comprise, at least in part, some reprogrammed cells, e.g., piPSC which are incomplete or not fully reprogrammed cells.

In some embodiments, a population of stable reprogrammed cells as disclosed herein can be used in kits. In some embodiments, the stable reprogrammed cells as disclosed herein can be in an admixture with other cells.

In some embodiments, a population of stable reprogrammed cells as disclosed herein can be used in a kit, for example to differentiate into a specific linage. In some embodiments, the stable reprogrammed cells as disclosed herein can be in an admixture with other cells. Another embodiments relates to a composition comprising a container, a population of stable reprogrammed cells and culture media. Other embodiments relate to a clonal cell line of a stable reprogrammed cell, e.g., piPS, or a stable reprogrammed cell isolated by the methods as disclosed herein.

In some embodiments, a stable reprogrammed cell, e.g., piPSC is a genetically modified piPSC.

In some embodiments, the stable reprogrammed cells as disclosed herein can be produced from the incomplete reprogramming of a somatic cell. In some embodiments, the somatic cell is a human cell. In some embodiments, a somatic cell is a diseased somatic cell, e.g., obtained from a subject with a pathology, or from a subject with a genetic predisposition to have, or be at risk of a disease or disorder. One can use any method for reprogramming a somatic cell, for example, as disclosed in International patent applications; WO2007/069666; WO2008/118820; WO2008/124133; WO2008/151058; WO2009/006997; and U.S. Patent Applications US2010/0062533; US2009/0227032; US2009/0068742; US2009/0047263; US2010/0015705; US2009/0081784; US2008/0233610; US7615374; U.S. patent application Ser. No. 12/595,041, EP2145000, CA2683056, AU8236629, Ser. No. 12/602,184, EP2164951, CA2688539, US2010/0105100; US2009/0324559, US2009/0304646, US2009/0299763, US2009/0191159, the contents of which are incorporated herein in their entirety by reference. In some embodiments, a stable reprogrammed cells disclosed herein can be produced by any method known in the art for reprogramming a cell, for example virally-induced or chemically induced generation of reprogrammed cells, as disclosed in EP1970446, US2009/0047263, US2009/0068742, and 2009/0227032, which are incorporated herein in their entirety by reference.

In some embodiments, the stable reprogrammed cells disclosed herein can be produced from the incomplete reprogramming of a somatic cell by chemical reprogramming, such as by the methods as disclosed in WO2010/033906, the contents of which is incorporated herein in its entirety by reference. In alternative embodiments, the stable reprogrammed cells disclosed herein can be produced from the incomplete reprogramming of a somatic cell by non-viral means, such as by the methods as disclose in WO2010/048567 the contents of which is incorporated herein in its entirety by reference.

In some embodiments, a population of stable reprogrammed cells as disclosed herein can be produced by reprogramming with synthetic mRNAs (also known as modified-RNA) which encode reprogramming transcription factors, according to the methods as disclosed in U.S. Provisional Application 61/387,220, filed Sep. 28, 2010, and U.S. Provisional Application 61/325,003, filed: Apr. 16, 2010, both of which are incorporated herein in their entirety by reference. Without wishing to be limited to theory, the term “synthetic RNA” or “modified RNA” are used interchangeably herein and refers to a nucleic acid molecule encoding a factor, such as a polypeptide, to be expressed in a host cell, which comprises at least one modified nucleoside and has at least the following characteristics as the term is used herein: (i) it can be generated by in vitro transcription and is not isolated from a cell; (ii) it is translatable in vivo in a mammalian (and preferably human) cell; and (iii) it does not provoke or provokes a significantly reduced innate immune response or interferon response in a cell to which it is introduced or contacted relative to a synthetic, non-modified RNA of the same sequence. A synthetic, modified-RNA as described herein permits repeated transfections in a target cell or tissue in vivo; that is, a cell or cell population transfected in vivo with a synthetic, modified-RNA molecule as described herein tolerates repeated transfection with such synthetic, modified-RNA without significant induction of an innate immune response or interferon response.

In some embodiments, the stable reprogrammed cells as disclosed herein can be produced by reprogramming with just small molecules or proteins. A large number of cocktails have been derived, all of which use different mixtures of small molecules and transduced genes (see Li et al., Trends Pharmacol. Sci., 2010, 31; 36-4, which is incorporated herein by reference). The small molecules identified have typically replaced one or more of the reprogramming factors or have improved the efficiency of the overall process. Many of the screens have provided some insight into the mechanism of reprogramming. In some embodiments, a population of stable reprogrammed cells as disclosed herein can be produced by reprogramming using chemicals, according to the methods as disclosed in WO 2010/033906 which is incorporated herein in its entirety by reference.

In some embodiments, the stable reprogrammed cells as disclosed herein can be further reprogrammed to a less differentiated cell, e.g., a fully reprogrammed iPS cell (where an iPS cell is terminally reprogrammed and cannot be further reprogrammed), by increasing the expression of a reprogramming factor in the stable intermediate piPS cell. In some embodiments, as disclosed herein, contacting the stable reprogrammed intermediate with a small molecule reprogramming factor, e.g., RepSox can reprogram a cell to a fully (terminally) reprogrammed cell, e.g., an iPS cell. In some embodiments, a small molecule which serves as a reprogramming factor is selected from the group consisting of: RepSox, 2i, 5′Ara C or any combination thereof.

In some embodiments, one can select for stable reprogrammed intermediate (piPS) cells which have the capacity for further reprogramming to an iPS cell (e.g., a fully reprogrammed cell which is terminally reprogrammed) by selecting for cells which have a higher level of H3K4me2 methylation of the Nanog proximal promoter by a statistically significant amount as compared to H3K27me2 methylation of the Nanog proximal promoter.

In some embodiments, a stable reprogrammed intermediate (piPS) cell as disclosed herein can be directly differentiated into any one of the following cell types: cardiomyocytes, neurons, motor neurons, neurectodermal precursors, definitive endoderm.

In some embodiments, a stable reprogrammed intermediate (piPS) cell can be classified based on one of three morphological and marker characteristics, as disclosed in Table 2, where the classification groups a reprogrammed cell into one of the following groups (i) alkaline phosphatase negative, SSEA1 negative, small granular colonies, (ii) alkaline phosphatase positive, SSEA1 negative, compact colonies, and (iii) alkaline phosphatase positive, SSEA1 positive, compact colonies. In some embodiments, one can select for a specific stable reprogrammed cell from one of the three groups of stable reprogrammed cells, based upon selecting for positive or negative expression of alkaline phostphatase expression and SSEA1, where reprogrammed cells can be isolated based (i) negative alkaline phosphatase expression, negative SSEA1 expression and optionally, small granular colonies, (ii) positive alkaline phosphatase expression, negative SSEA1 expression and optionally compact colonies, and (iii) positive alkaline phosphatase and SSEA1 expression and optionally compact colonies.

In some embodiments, the stable reprogrammed cells, e.g., piPSC can be differentiated into definitive endoderm. Methods for producing definitive endoderm cells are known in the art, including, for example the methods which are set forth in United States application publication US2006/0003446 to G. Keller, et al.; US2006/0003313 to K. D'Amour, et al., US2005/0158853 to K. D'Amour, et al., and US2005/0260749 of Jon Odorico, et al., relevant portions of which are incorporated by reference herein.

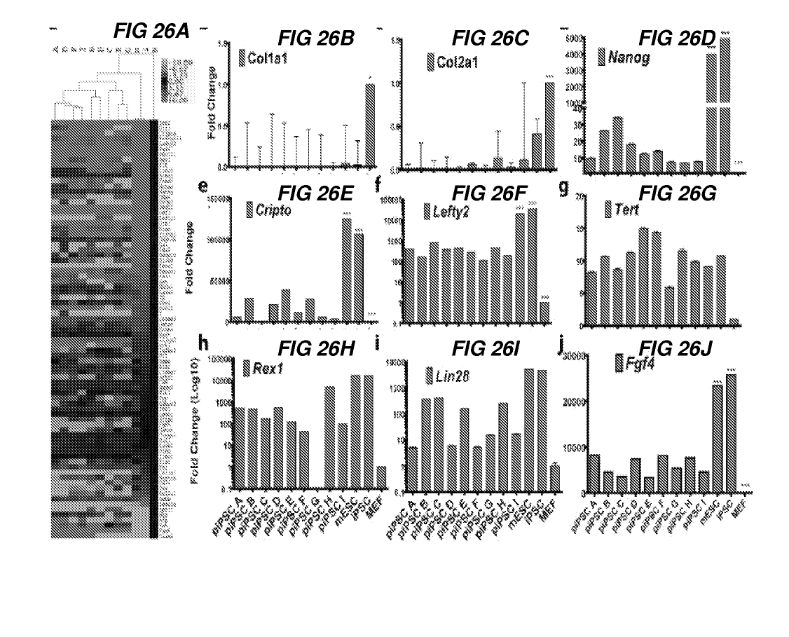

In some embodiments, a population of stable reprogrammed cells as disclosed herein can be selected and isolated from a population of cells which has undergone the completion of a reprogramming protocol (e.g., the reprogramming protocol is followed through to completion or to the end). Thus, in some embodiments, the stable reprogrammed cells can be selected and isolated from a mixed population of cells comprising both completely reprogrammed cells (e.g., iPSCs) and partially stable reprogrammed cells (e.g., piPSCs) as disclosed herein. In some embodiments, such a mixed population used for piPSCs selection and isolation can comprise both completely reprogrammed cells (e.g., iPSCs) and partially stable reprogrammed cells (e.g., piPSCs), and can in some embodiments, can comprises at least about 20%, or at least about 30%, or at least 40% or at least 50% or more completely reprogrammed cells (e.g., iPSCs) or alternatively, comprises at least about 1%, or at least about 5%, or at least about 10%, or at least about 20%, or at least about 30%, or at least 40% or at least 50% or more cells which express high levels of Nanog (e.g., express greater than 2000-fold of Nanog as compared to the starting somatic cell population, see FIG. 26D).

As described herein, a population of cells (e.g., somatic cells) has undergone completion of a reprogramming protocol after exposure to one or more reprogramming agents in the reprogramming protocol refers to at least one cell of the cell population has been completely reprogrammed, e.g., expresses a high level of Nanog (e.g., express greater than 2000-fold of Nanog as compared to the starting somatic cell population from which the cell was derived, e.g., see FIG. 26D). In one embodiment, at least about 0.01% or at least about 0.05%, or at least 0.1% or at least 0.5% or at least about 1% or more cells have undergone complete reprogramming and become iPSCs which express high levels of Nanog (e.g., express greater than 2000-fold of Nanog as compared to the starting somatic cell population, see FIG. 26D).

In alternative embodiments, a population of stable reprogrammed cells as disclosed herein can be selected and isolated from a population of cells in which the reprogramming protocol is aborted or stopped before completion of the full reprogramming protocol required for obtaining complete reprogramming of cells (e.g., to produce iPSCs). For example, in some embodiments, one can perform a reprogramming protocol and part-way-through (e.g., before the end of the protocol or before complete reprogramming the cells to iPSCs), the cells are transferred from an ES-like media to a differentiation media. Accordingly, in some embodiments, a population of stable reprogrammed cells as disclosed herein can be produced by a truncated reprogramming protocol where complete of reprogramming of some cells to iPSCs is not achieved, thereby only reprogramming the cells partially to produce a population of stable partially reprogrammed cells as disclosed herein. In such an embodiment, one can truncate or prevent complete reprogramming of cells to iPSCs by transferring the cells to a differentiation media after a predetermined amount of time, according to the methods as disclosed in Efe et al., “Conversion of mouse fibroblasts into cardiomyocytes using direct repramming strategy, Nat. Cell Biol., 2011; 13:215-22, which is incorporated herein in its entirety by reference. In some embodiments, a population of cells which has undergone a trunkated reprogramming protocol comprises less than 0.1%, or less than 0.01% or less than 0.001% completely reprogrammed cells, e.g., iPSCs, or comprises less than 10%, or less than 5% or less than 2% cells which express high levels of Nanog (e.g., express greater than 2000-fold of Nanog as compared to the starting somatic cell population, see FIG. 26D).

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “reprogramming” as used herein refers to a process that alters or reverses the differentiation state of a differentiated cell (e.g. a somatic cell). Stated another way, reprogramming refers to a process of driving the differentiation of a cell backwards to a more undifferentiated or more primitive type of cell. Complete reprogramming involves complete reversal of at least some of the heritable patterns of nucleic acid modification (e.g., methylation), chromatin condensation, epigenetic changes, genomic imprinting, etc., that occur during cellular differentiation as a zygote develops into an adult. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods of the invention may also be of use for such purposes.

The term “stable reprogrammed cell” as used herein refers to a cell which is produced from the partial or incomplete reprogramming of a differentiated cell (e.g. a somatic cell). A stable reprogrammed cell is used interchangeably herein with “piPSC”. A stable reprogrammed cell has not undergone complete reprogramming and thus has not had global remodeling of the epigenome of the cell. In one embodiment, a piPSC is a cell which has not been reprogrammed to an embryonic-like state. A stable reprogrammed cell is a pluripotent stem cell and can be further reprogrammed to an iPSC, as that term is defined herein, or alternatively can be differentiated along different lineages. In some embodiments, a partially reprogrammed cell expresses markers from all three embryonic germ layers (i.e. all three layers of endoderm, mesoderm or ectoderm layers). Markers of endoderm cells include, Gata4, FoxA2, PDX1, Nodal, Sox7 and Sox17. Markers of mesoderm cells include, Brachycury, GSC, LEF1, Mox1 and Tie1. Markers of ectoderm cells include criptol, EN1, GFAP, Islet 1, LIM1 and Nestin. In some embodiments, a partially reprogrammed cell is an undifferentiated cell.

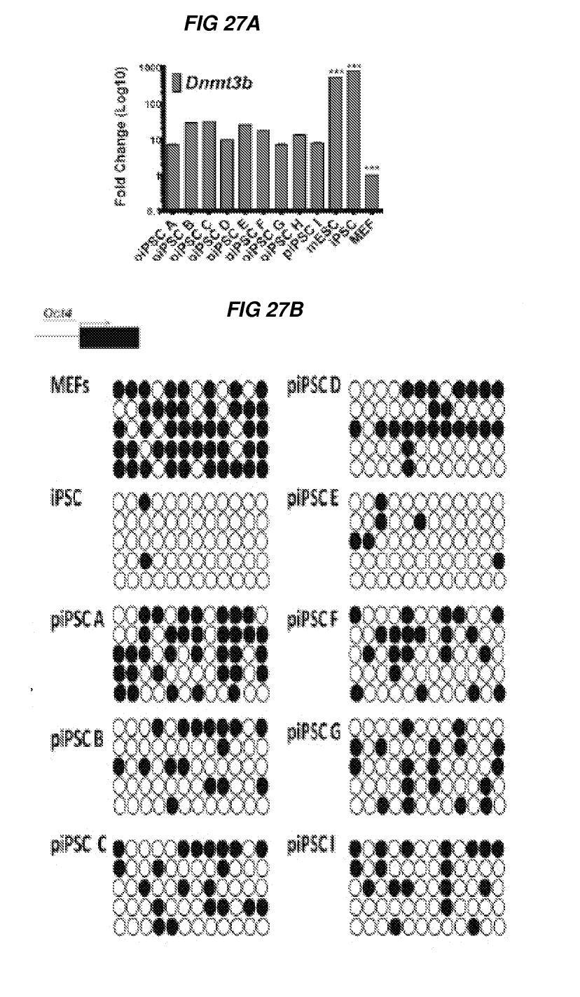

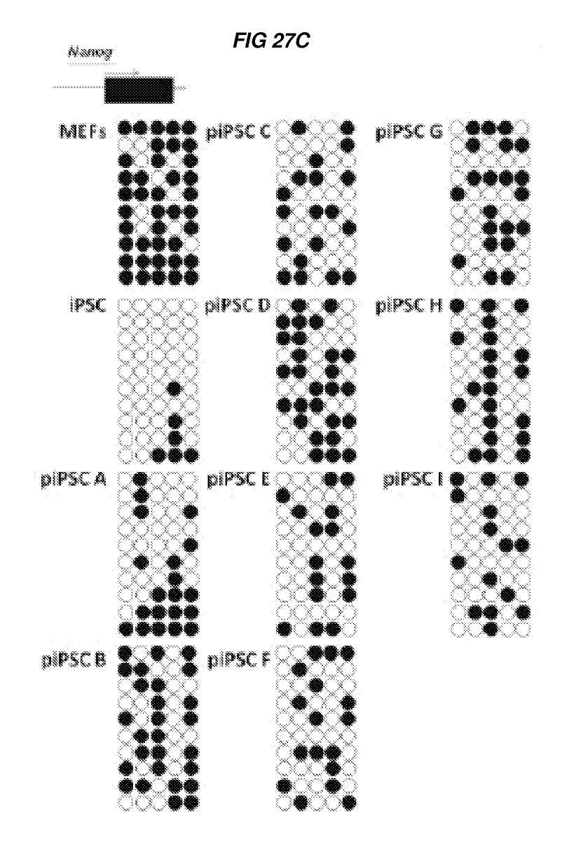

The term “induced pluripotent stem cell” or “iPSC” or “iPS cell” refers to a cell derived from a complete reversion or reprogramming of the differentiation state of a differentiated cell (e.g. a somatic cell). As used herein, an iPSC is fully reprogrammed and is a cell which has undergone complete epigenetic reprogramming. As used herein, an iPSC is a cell which cannot be further reprogrammed (e.g., an iPSC cell is terminally reprogrammed). In one embodiment, complete reversion is determined by the lack of expression of Xist or a complete level of CGp methylation. In some embodiments, a fully reprogrammed cell completely expresses Nanog at high levels, for example an expression greater than 2000-fold of Nanog as compared to the starting somatic cell population (see FIG. 26D). In some embodiments, a fully reprogrammed cell completely expresses Dnmt3b at high levels, for example an expression greater than 100-fold of Dnmt3b as compared to the starting somatic cell population (see FIG. 27A). In some embodiments, a fully reprogrammed cell completely expresses Fgf4 at high levels, for example an expression greater than 2000-fold of Fgf4 as compared to the starting somatic cell population (see FIG. 26J). In some embodiments, complete reprogramming or reversion is determined by Oct4 and/or nanog promoter CGp methylation (e.g., see FIG. 27B-27C), where fully reprogrammed cells do have very low levels or absence of Oct4 and/or Nanog promoter methylation. In one embodiment, an iPSC is a cell in an embryonic-like state.

The term “remodeling of the epigenome” refers to chemical modifications of the genome which do not change the genomic sequence or a gene's sequence of base pairs in the cell, but alter the expression.

The term “global remodeling of the epigenome” refers to where chemical modifications of the genome have occurred where there is no memory of prior gene expression from the differentiated cell from which the reprogrammed cell or iPSC was derived.

The term “incomplete remodeling of the epigenome” refers to where chemical modifications of the genome have occurred where there is memory of prior gene expression from the differentiated cell from which the stable reprogrammed cell or piPSC was derived.

The term “epigenetic reprogramming” as used herein refers to the alteration of the pattern of gene expression in a cell via chemical modifications that do not change the genomic sequence or a gene's sequence of base pairs in the cell.

The term “epigenetic” as used herein refers to “upon the genome”. Chemical modifications of DNA that do not alter the gene's sequence, but impact gene expression and may also be inherited. Epigenetic modification to DNA are important in imprinting and cellular reprogramming.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent cell is an undifferentiated cell.

The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult stem cells, such as for example, hematopoietic stem cells and neural stem cells. Multipotent means a stem cell may form many types of cells in a given lineage, but not cells of other lineages. For example, a multipotent blood stem cell can form the many different types of blood cells (red, white, platelets, etc. . . . ), but it cannot form neurons.

The term “multipotency” refers to a cell with the degree of developmental versatility that is less than totipotent and pluripotent.

The term “totipotency” refers to a cell with the degree of differentiation describing a capacity to make all of the cells in the adult body as well as the extra-embryonic tissues including the placenta. The fertilized egg (zygote) is totipotent as are the early cleaved cells (blastomeres)

The term “differentiated cell” is meant any primary cell that is not, in its native form, pluripotent as that term is defined herein. The term a “differentiated cell” also encompasses cells that are partially differentiated, such as multipotent cells, or cells that are stable non-pluripotent partially reprogrammed cells. It should be noted that placing many primary cells in culture can lead to some loss of fully differentiated characteristics. Thus, simply culturing such cells are included in the term differentiated cells and does not render these cells non-differentiated cells (e.g. undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture. In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when an differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., meneghel-Rozzo et al., (2004), Cell Tissue Res, 316(3); 295-303, which is incorporated herein in its entirety by reference.

As used herein the term “isogenic cell” refers to a cell originating from a common source or having the same genetic makeup. For example, when comparing an iPSC and a stable reprogrammed cell, e.g., piPSC as disclosed herein, an isogenic iPSCs, piPSC, or somatic cell are cells which all originate from the same species, e.g. human and in some embodiments, from the same organism. In some embodiments, for the purposes of comparison of a stable reprogrammed cell (piPSC) as disclosed herein with an induced pluripotent stem cell (e.g., iPSC), the cells are derived from the same type of somatic cell or cells (e.g., epithelial cells, fibroblasts cells, neuronal cells, etc.) and are isogenic.

As used herein, the term “adult cell” refers to a cell found throughout the body after embryonic development.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell), which in turn can differentiate into other types of precursor cells further down the pathway (such as an tissue specific precursor, for example, a cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “phenotype” refers to one or a number of total biological characteristics that define the cell or organism under a particular set of environmental conditions and factors, regardless of the actual genotype.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “exogenous” refers to a substance present in a cell other than its native source. The terms “exogenous” when used herein refers to a nucleic acid (e.g. a nucleic acid encoding a sox2 transcription factor) or a protein (e.g., a sox2 polypeptide) that has been introduced by a process involving the hand of man into a biological system such as a cell or organism in which it is not normally found or in which it is found in lower amounts. A substance (e.g. a nucleic acid encoding a sox2 transcription factor, or a protein, e.g., a sox2 polypeptide) will be considered exogenous if it is introduced into a cell or an ancestor of the cell that inherits the substance. In contrast, the term “endogenous” refers to a substance that is native to the biological system or cell (e.g. differentiated cell).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of reprogrammed cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not reprogrammed cells or their progeny as defined by the terms herein. In some embodiments, the present invention encompasses methods to expand a population of reprogrammed cells, wherein the expanded population of reprogrammed cells is a substantially pure population of reprogrammed cells.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and refers to a process of a cell making more copies of itself (e.g. duplication) of the cell. In some embodiments, reprogrammed cells are capable of renewal of themselves by dividing into the same undifferentiated cells (e.g. pluripotent or non-specialized cell type) over long periods, and/or many months to years. In some instances, proliferation refers to the expansion of reprogrammed cells by the repeated division of single cells into two identical daughter cells.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The term “lineages” as used herein describes a cell with a common ancestry or cells with a common developmental fate. By way of an example only, a cell that is of endoderm origin or is “endodermal linage” this means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “DNA” is defined as deoxyribonucleic acid.

The term “differentiation” as used herein refers to the cellular development of a cell from a primitive stage towards a more mature (i.e. less primitive) cell.

The term “directed differentiation” as used herein refers to forcing differentiation of a cell from an undifferentiated (e.g. more primitive cell) to a more mature cell type (i.e. less primitive cell) via genetic and/or environmental manipulation. In some embodiments, a reprogrammed cell as disclosed herein is subject to directed differentiation into specific cell types, such as neuronal cell types, muscle cell types and the like.

The term “functional assay” as used herein is a test which assesses the properties of a cell, such as a cell's gene expression or developmental state by evaluating its growth or ability to live under certain circumstances. In some embodiments, a reprogrammed cell can be identified by a functional assay to determine the reprogrammed cell is a pluripotent state as disclosed herein.

The term “disease modeling” as used herein refers to the use of laboratory cell culture or animal research to obtain new information about human disease or illness. In some embodiments, a reprogrammed cell produced by the methods as disclosed herein can be used in disease modeling experiments.

The term “drug screening” as used herein refers to the use of cells and tissues in the laboratory to identify drugs with a specific function. In some embodiments, the present invention provides drug screening methods of differentiated cells to identify compounds or drugs which reprogram a differentiated cell to a reprogrammed cell (e.g. a reprogrammed cell which is in a pluripotent state or a reprogrammed cell which is a stable intermediate, partially reprogrammed cell, as disclosed herein). In some embodiments, the present invention provides drug screening methods of stable intermediate partially reprogrammed cells to identify compounds or drugs which reprogramming differentiated cells into fully reprogrammed cells (e.g. reprogrammed cells which are in a pluripotent state). In alternative embodiments, the present invention provides drug screening on reprogrammed cells (e.g. human reprogrammed cells) to identify compounds or drugs useful as therapies for diseases or illnesses (e.g. human diseases or illnesses).

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, e.g., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The term “retrovirus” as used herein refers to a specific type of virus with a RNA-genome that can be engineered to intergrate new genetic material into host target cells.

The term “infection” as used herein refers to expose target cells to a mixture of viral particles that contain new genetic material one wishes to functionally evaluate

The term “lentivirus” as used herein refers to a specific type of virus with an RNA genome (such as HIV) that can be engineered to deliver and integrate new genetic material into target cells. Lentivirus has certain advantages over other retroviruses including that it can deliver its genetic payload to the nucleus of non-dividing target cells. The term “transcriptional profile” as used herein refers to the state of gene expression in a given cell or tissue type

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. In some embodiments, the term “treatment” can also mean prolonging survival as compared to expected survival if not receiving treatment. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management. The subject is usually ill or injured, or at an increased risk of becoming ill relative to an average member of the population and in need of such attention, care, or management.

As used herein, the terms “administering,” “introducing” and “transplanting” are used interchangeably in the context of the placement of reprogrammed cells as disclosed herein, or their differentiated progeny into a subject, by a method or route which results in at least partial localization of the reprogrammed cells, or their differentiated progeny at a desired site. The reprogrammed cells, or their differentiated progeny can be administered directly to a tissue of interest, or alternatively be administered by any appropriate route which results in delivery to a desired location in the subject where at least a portion of the reprogrammed cells or their progeny or components of the cells remain viable. The period of viability of the reprogrammed cells after administration to a subject can be as short as a few hours, e.g. twenty-four hours, to a few days, to as long as several years.

The term “transplantation” as used herein refers to introduction of new cells (e.g. reprogrammed cells), tissues (such as differentiated cells produced from reprogrammed cells), or organs into a host (i.e. transplant recipient or transplant subject)

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and the include plural references unless the context clearly dictates otherwise. Thus for example, references to “the method” includes one or more methods, and/or steps of the type described herein and/or which will become apparent to those persons skilled in the art upon reading this disclosure and so forth.

General Methods for Identifying Stable Partially Reprogrammed Cells (piPSCs) as Disclosed Herein.

Isolation of a Stable Reprogrammed Cell

One aspect relates to methods for the isolation of a stable reprogrammed cell, as disclosed herein. In some embodiments, one can perform the method of reprogramming a somatic cell as disclosed herein, and then make a single cell suspension and plate each cell individually to generate a plurality of different reprogrammed cell lines. In some embodiments, each reprogrammed cell line can be plated in number of replicate plates which can be used analysis, and if the plate is identified to contain a stable partially reprogrammed cell as disclosed herein, one can select the corresponding cell line colony in the original plate for further analysis. For example, a population of somatic cells can be reprogrammed using methods commonly known by persons of ordinary skill in the art, and the reprogrammed cell population plated as single cell cultures in an original multiwell plate. In some embodiments, the original multiwell plate is cultured and then used to generate replicate plates of the original multiwell plate. These can be used for methylation analysis and/or gene expression analysis according to the methods as disclosed herein. In some embodiments, where a reprogrammed cell line has a gene expression profile and/or methylation profile of a stable partially reprogrammed cell line as disclosed herein, one can select that piPS for further analysis or for any use, e.g., for differentiating into a desired cell lineage, for use in assays, or for therapeutic use.

As disclosed herein, a stable partially reprogrammed cell line as disclosed herein to be selected and isolated can have the gene expression profile of any combination of number of genes, selected from, but not limited to, Dnmt3b, Oct4, Tdgf1, Sox2, Col1a1, Col2a1, Nanog, Cripto, Lefty2, tert, Rex1, Lin 28, Fgf4, Krt10, thy1, Xist. In some embodiments, a stable partially reprogrammed cell line as disclosed herein to be selected for isolation based on the expression of at least 3, or at least 4, or at least 5 or at least 6, or more than 6 gene expression profiles selected from the group selected from Dnmt3b, Oct4, Tdgf1, Sox2, Col1a1, Col2a1, Nanog, Cripto, Lefty2, tert, Rex1, Lin 28, Fgf4, Krt10, thy1, Xist.

In some embodiments, a stable partially reprogrammed cell line as disclosed herein can be selected for isolation based on the gene expression profiles of Col1a1 expression, where a stable reprogrammed cell is selected which expresses at least about 0.5-fold, or at least about 0.7-fold or at least about 1-fold less expression of Col1a1 as compared to a MEF or the isogenic cell from which the stable reprogrammed cell was derived.

In some embodiments, a stable partially reprogrammed cell line as disclosed herein can be selected for isolation based on the gene expression profiles of Col2a1 expression, where a stable reprogrammed cell is selected which expresses at least about 0.2-fold, or at least about 0.3-fold or at least about 5-fold less expression of Col2a1 as compared to a MEF or the or a fully reprogrammed (iPSC).

In some embodiments, a stable partially reprogrammed cell line as disclosed herein can be selected for isolation based on the gene expression profiles of Cripto expression, where a stable reprogrammed cell is selected which expresses at least about 50,000-fold, or at least about 70,000-fold or at least about 100,000-fold less expression of Cripto as compared to a mESC or fully reprogrammed iPSC cells or an isogenic iPSC.

In some embodiments, an isolated stable reprogrammed cell can be selected which a lower expression of endogenous Lefty 2 by a statistically significant amount relative to the level of expression of an induced pluripotent stem (iPS) cell, for example, at least a 100-fold lower expression, or at least a 1000-fold lower expression, or at least a 10,000-fold lower expression, or anywhere between about 15,000-fold and 10,000-fold lower expression of endogenous Lefty 2 as compared to the level of expression of an induced pluripotent stem (iPS) cell.

In some embodiments, an isolated stable reprogrammed cell can be selected which has a higher level of expression of any one or more of endogenous Tdgf1, endogenous Tert or endogenous Sox2 by a statistically significant amount relative to the level of expression of a somatic cell, for example the somatic cell from which it was derived.

In some embodiments, an isolated stable reprogrammed cell can be selected for isolation which has at least a 1,000-fold higher expression of endogenous Tgdf1 as compared to the level of expression of endogenous Tgdf1 in a somatic cell, for example, at least a 5,000-fold higher expression, or at least about a 7,000-fold higher expression, or anywhere between about a 7,000-fold to 15,000-fold higher expression of endogenous Tgdf1 as compared to the level of expression of endogenous Tgdf1 in a somatic cell.

In some embodiments, an isolated stable reprogrammed cell can be selected for isolation which has at least a 5-fold higher expression of endogenous Tert as compared to the level of expression of endogenous Tert in a somatic cell, for example, at least a 7-fold higher expression, or between a 7-fold and 20-fold higher expression of endogenous Tert as compared to the level of expression of endogenous Tert in a somatic cell.

In some embodiments, an isolated stable reprogrammed cell can be selected for isolation which has at least a 10,000-fold lower expression of endogenous Fgf4t as compared to the level of expression of endogenous Fgf4 in a fully reprogrammed stem cell (e.g. iPSC) or mESC, for example, at least a 10,000-fold lower expression, or between a 12,000-fold and 15,000-fold lower expression of endogenous Fgf4 as compared to the level of expression of endogenous Fgf4 in a fully reprogrammed stem cell (e.g. iPSC), somatic cell or mESC.

In some embodiments In some embodiments, a stable partially reprogrammed cell line as disclosed herein to be selected for isolation based on the gene expression profiles of Rest expression, where a stable reprogrammed cell is selected which expresses at least about 10-fold, or at least about 15-fold or at least about 20 fold less Rest gene expression as compared to a fully isolated iPSC or mESC.

In some embodiments, an isolated stable reprogrammed cell can be selected for isolation which has at least a 100-fold higher expression of endogenous Sox2 as compared to the level of expression of endogenous Sox2 in a somatic cell, for example, at least a 500-fold higher expression, or between about a 500-fold and 100,000-fold higher expression, or between about a 500-fold and 10,000-fold higher expression of endogenous Sox2 as compared to the level of expression of endogenous Sox2 in a somatic cell.

In some embodiments, an isolated stable reprogrammed cell can be selected for isolation which has a lower expression of Krt10 or Thy1 or Krt10 and Thy1 by a statistically significant amount relative to the level of expression of the isogenic cell from which the reprogrammed cell was derived.

piPSC lines exhibit heterogeneous expression of early reprogramming markers and distinct proliferative rates. To characterize differences between piPSC and iPSC, the inventors first immunostained for the early reprogramming markers alkaline phosphatase (AP) and SSEA1. Using these markers, piPSC lines can be isolated based on the three expression and morphological profiles of the early reprogramming markers SSEA1 and AP, as disclosed in Table 2.

One aspect of the present invention relates to an isolated reprogrammed cell induced from a differentiated cell, wherein the cell has been reprogrammed to a less differentiated state and can form colonies with distinct morphologies and is capable of self renewing for at least twenty passages before senescence. In some embodiments, the isolated reprogrammed cell can differentiate into all three primary germ layer lineages selected from; endoderm lineage, mesoderm lineage and ectoderm lineage.

In some embodiments, the isolated reprogrammed cell can be selected for isolation based on a doubling time of less than 15 hours, for example between 15 and 5 hours, and in some embodiments, a reprogrammed cell can be selected based on a significantly faster rate of doubling time as compared to a completely reprogrammed iPS cell, for example, a doubling time of at least 1.5 fold faster as compared to an induced pluripotent stem (iPS) cell, or at least about 2-fold faster as compared to an induced pluripotent stem (iPS) cell.

In some embodiments, an isolated stable reprogrammed cell can be selected for isolation based on a lower expression of Dnmt3b by a statistically significant amount relative to the level of expression of an induced pluripotent stem (iPS) cell, such as at least a 100-fold lower expression, or at least a 200-fold lower expression, or at least a 500-fold lower expression of Dnm3b as compared to the level of expression of an induced pluripotent stem (iPS) cell.

In some embodiments, an isolated stable reprogrammed cell can be selected for isolation based on a lower expression of at least 2 of the following genes; endogenous Oct4, endogenous Nanog, endogenous Rex1, endogenous Tdgf1 by a statistically significant amount relative to the level of expression of an induced pluripotent stem (iPS) cell.

In some embodiments, an isolated stable reprogrammed cell can be selected for isolation based on at least a 100-fold lower expression of endogenous Nanog as compared to the level of expression of an induced pluripotent stem (iPS) cell, for example, at least a 1000-fold lower expression, or at least a 5000-fold lower expression, or about or more than a 10,000-fold lower expression of endogenous Nanog as compared to the level of expression of an induced pluripotent stem (iPS) cell.

In some embodiments, an isolated stable reprogrammed cell can be selected for isolation based on an increased CpG methylation of Oct4 or Nanog or Oct4 and Nanog a statistically significant amount relative to the level of CpG methylation of Oct4 or Nanog or Oct4 and Nanog an induced pluripotent stem (iPS), and/or a decreased CpG methylation of Oct4 or Nanog or Oct4 and Nanog a statistically significant amount relative to the level of CpG methylation of Oct4 or Nanog or Oct4 and Nanog an the somatic cell, such as an isogenic cell from which the reprogrammed cell was derived.

In some embodiments, an isolated stable reprogrammed cell can be selected for isolation based on an increased rate of proliferation by a statistically significant amount relative to the level of expression of an induced pluripotent stem (iPS) cell.

In some embodiments, an isolated stable reprogrammed cell can be selected for isolation based on an increased expression of Xist in female piPS lines by a statistically significant amount to the level of expression of the somatic cell from which the reprogrammed cell was derived, for example, at least a 5-fold higher expression of Xist, or between about a 5-fold and about a 50-fold higher expression of Xist, or between about a 5-fold and about a 20-fold higher expression of Xist in female lines as compared to the level of expression of endogenous Xist in a female induced pluripotent stem (iPS) cell.

In some embodiments, an isolated stable reprogrammed cell can be selected or isolation if the stable reprogrammed cell (e.g. piPSC) can be further reprogrammed to a more undifferentiated state.

In some embodiments, an isolated stable reprogrammed cell can be selected for isolation based on incomplete remodeling of the epigenome. In some embodiments, an isolated stable reprogrammed cell can be selected for isolation based on at least about 10%, or at least about 20%, or at least about 30%, or at least about 40%, or at least about 50%, or at least about 60%, or at least about 70%, or more than 70% less remodeling of the epigenome as compared to an induced pluripotent stem (iPS) cell. Stated another way, where an iPSC has undergone global or 100% remodeling of the eipgenome, a stable reprogrammed cell as disclosed herein, e.g. a piPSC, can be selected for isolation where the epigenome has undergone less than 100% remodeling, for example less than 90%, or less than 80%, or less than about 70%, or less than about 60%, or less than about 50%, or less than about 40%, or less than about 30%, or less than about 20%, or less than about 10% of the epigenome is remodeled.

In some embodiments, an isolated stable reprogrammed cell can be selected for isolation if the cell can be further reprogrammed into an induced pluripotent stem (iPS) cell.

In some embodiments, an isolated stable reprogrammed cell can be selected for isolation based on at least a 10-fold lower, or at least about 15-fold lower expression of Dnmt3b relative to the level of expression of an induced pluripotent stem (iPS) cell.

In some embodiments, an isolated stable reprogrammed cell can be selected for isolation based upon ability for multilineage differentiation, for example, differentiation into one or more of the following linages; neuronal lineages, mesoderm and ectoderm lineages. In some embodiments, the efficiency of differentiation along different lineages differs between clones of piPSC as disclosed herein in the Drawings. In some embodiments, some reprogrammed cells differentiate along neuronal lineages with greater efficiency than other reprogrammed cells.

In some embodiments, an isolated stable reprogrammed cell can be selected for isolation which differentiate along neuronal lineages, for example, a reprogrammed cell can differentiate along neuronal lineages at a significantly higher efficiency than that of an induced pluripotent stem (iPS) cell.

In some embodiments, an isolated stable reprogrammed cell can be selected for isolation which differentiate along cardiogenic lineages, for example, a reprogrammed cell can differentiate along cardiac lineages at a significantly higher efficiency than that of an induced pluripotent stem (iPS) cell.

In some embodiments, one can select for stable reprogrammed intermediate (piPS) cells which have the capacity for further reprogramming to an iPS cell (e.g., a fully reprogrammed cell which is terminally reprogrammed) by selecting for cells which have a higher level of H3K4me2 methylation of the Nanog proximal promoter by a statistically significant amount as compared to H3K27me2 methylation of the Nanog proximal promoter.

In some embodiments, a stable reprogrammed intermediate (piPS) cell can be selected based on one of three morphological and marker characteristics, as disclosed in Table 2, where the classification groups a reprogrammed cell into one of the following groups (i) alkaline phosphatase negative, SSEA1 negative, small granular colonies, (ii) alkaline phosphatase positive, SSEA1 negative, compact colonies, and (iii) alkaline phosphatase positive, SSEA1 positive, compact colonies. In some embodiments, one can select for a specific stable reprogrammed cell from one of the three groups of stable reprogrammed cells, based upon selecting for positive or negative expression of alkaline phostphatase expression and SSEA1, where reprogrammed cells can be isolated based (i) negative alkaline phosphatase expression, negative SSEA1 expression and optionally, small granular colonies, (ii) positive alkaline phosphatase expression, negative SSEA1 expression and optionally compact colonies, and (iii) positive alkaline phosphatase and SSEA1 expression and optionally compact colonies.

In some embodiments, a stable reprogrammed intermediate (piPS) cell as disclosed herein can be selected for based on a particular characteristic profile as determined by a stem cell scorecard as disclosed in Bock et al., Cell, 2011, 114; 439-52, and disclosed in U.S. provisional patent applications 61/384,030 filed on Sep. 17, 2010 and U.S. provisional patent application 61/429,965 filed on Jan. 5, 2011, which are both incorporated herein in their entirety by reference.

Accordingly, in some embodiments, one can select for a population of stable reprogrammed intermediate (piPS) cells based on a particular profile, e.g., predisposition to differentiate along a desired lineage or deviation of values from other piPSC. Without being limited to theory, Bock and colleagues (Bock et al., Cell, 2011, 114; 439-52) carried out an extensive bioinformatics comparison of human iPSC and human ESC lines including DNA methylation patterns, microarray analyses, and a general differentiation assay in which gene expression was analyzed in embryoid bodies derived from each line. On the basis of these data, it was possible to distinguish an average iPSC line from an average ESC line. Importantly, the authors developed a scorecard based on a 500-gene expression array to quantify the differentiation tendencies of each line. The scorecard predicted that two of the iPSC lines might have reduced ability to differentiate into neurons and this was confirmed experimentally in a study carried out by Boulting and colleagues, who also showed that most iPSC lines, whether derived from healthy controls of different ages and sexes or from different types of ALS patients, could be induced to differentiate adequately into motor neurons. Accordingly, in some embodiments, a stable reprogrammed intermediate (piPS) cell as disclosed herein can be selected based on a particular profile of scorecard values according to the methods as disclosed in U.S. provisional patent applications 61/384,030 (filed on Sep. 17, 2010) and 61/429,965 (filed on Jan. 5, 2011), which are both incorporated herein in their entirety by reference.

Methods for Measuring DNA Methylation

While not wishing to be bound by theory, epigenetic events play a significant role in the expression of genes. Epigenetic changes such as DNA methylation act to regulate gene expression in normal mammalian development. The term “epigenetics” refers to heritable changes in gene expression that do not result from alterations in the gene nucleotide sequence. For example, when DNA is methylated in the promoter region of genes, where transcription is initiated, genes are inactivated and silenced resulting in no or low expression of the gene. Epigenetic modification includes for example, without limitation, DNA methylation, posttranslational modification of chromatin, small non-coding RNA's, and non-covalent structural modifications to chromatin, such as condensation and decondensation of chromatin.

Promoter hypermethylation also plays a major role in cancer through transcriptional silencing of critical growth regulators such as tumor suppressor genes. Loss of function of genes, such as tumor suppressor genes can occur through epigenetic changes such as DNA methylation.

Numerous methods for analyzing methylation status of a gene are known in the art and can be used in the methods of the present invention to identify either hypomethylation or hypermethylation of the one or more promoter regions. In various embodiments, the determining of methylation status in the methods of the invention is performed by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, microarray technology, and proteomics or as methods as disclosed in International Application WO2011/046635, which is incorporated herein in its entirety by reference. As illustrated in the Examples herein, analysis of methylation can be performed by bisulfite genomic sequencing. Bisulfite treatment modifies DNA converting unmethylated, but not methylated, cytosines to uracil. Bisulfite treatment can be carried out using the METHYLEASY bisulfite modification kit (Human Genetic Signatures).

In some embodiments, bisulfite pyrosequencing, which is a sequencing-based analysis of DNA methylation that quantitatively measures multiple, consecutive CpG sites individually with high accuracy and reproducibility, may be used. Exemplary primers for such analysis are set forth in Table 2.

It will be recognized that depending on the site bound by the primer and the direction of extension from a primer, that the primers listed above can be used in different pairs. Furthermore, it will be recognized that additional primers can be identified within the promoter regions, especially primers that allow analysis of the same methylation sites as those analyzed with primers that correspond to the primers disclosed herein.

Altered methylation can be identified by identifying a detectable difference in methylation. For example, hypomethylation can be determined by identifying whether after bisulfite treatment a uracil or a cytosine is present a particular location. If uracil is present after bisulfite treatment, then the residue is unmethylated. Hypomethylation is present when there is a measurable decrease in methylation.

In an alternative embodiment, the method for analyzing methylation of a promoter region can include amplification using a primer pair specific for methylated residues within a promoter region. In these embodiments, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

One can use any method to measure DNA methylation which is commonly known to persons of ordinary skill in the art, including, but not limited to, enrichment-based methods (e.g. MeDIP, MBD-seq and MethylCap), bisulfite-based methods (e.g. RRBS, bisulfite sequencing, Infinium, GoldenGate, COBRA, MSP, MethyLight) and restriction-digestion methods (e.g., MRE-seq). In one embodiment, a method for epigenetic profiling and epigenetic mapping is whole genome epigenetic mapping. One can use any method for epigenetic mapping of a pluripotent stem cell line known to one of ordinary skill in the art, and includes, for example reduced-representation bisulfite sequencing (RRBS), as well as methods disclosed in U.S. Patent Application US2010/0172880, which is incorporated herein in its entirety by reference. Other DNA methylation assays are disclosed in U.S. Application US2008/0213789 and US2010/0075331 and in U.S. Pat. Nos. 6,960,434 and 7,425,415, which are incorporated herein in their entirety by reference. Method for measuring DNA methylation of pluripotent stem cells is also described in “Genome-wide mapping of DNA methylation: a quantitative technology comparison” by Bock et al., which is incorporated herein in its entirety by reference, where the inventors evaluated a variety of DNA methylation methods (MeDIP-seq: methylated DNA immunoprecipitation, MethylCap-seq: methylated DNA capture by affinity purification, RRBS: reduced representation bisulfite sequencing, and the Infinium HumanMethylation assay) produce accurate DNA methylation data of pluripotent stem cells.

In some embodiments, the DNA methylation assays are species-specific, so the use of mouse embryonic fibroblasts as a feeder layer for human pluripotent stem cells will not interfere with the epigenetic analysis.

Several methods have been developed to enable DNA methylation profiling on a genomic scale. Most of these methods combine DNA analysis by microarrays or high-throughput sequencing with one of four ways of translating DNA methylation patterns into DNA sequence information or library enrichment: (i) Methylated DNA immunoprecipitation (MeDIP) uses an antibody that is specific for 5-methyl-cytosine to retrieve methylated fragments from sonicated DNA11, (ii) Methylated DNA capture by affinity purification (MethylCap) employs a methyl-binding domain protein to obtain DNA fractions with similar methylation levels. (iii) Bisulfite-based methods utilize a chemical reaction that selectively converts unmethylated (but not methylated) cytosines into uracils, thus introducing methylation-specific single-nucleotide polymorphisms into the DNA sequence. (iv) Methylation-specific digestion uses prokaryotic restriction enzymes to fractionate DNA in a methylation-specific way.

Four popular methods, with a special emphasis on their practical utility for biomedical research and biomarker development were assessed previously by the inventors, which included MeDIP-seq, MethylCap-seq, RRBS and the Infinium HumanMethylation assay, (see “Genome-wide mapping of DNA methylation: a quantitative technology comparison” by Bock et al.). These methods are useful in the methods, systems and assays of the present invention, based on the following considerations: (i) All four methods are relatively easy to set up because detailed protocols have been published and/or commercial kits are available. (ii) RRBS has an advantage over other genome-wide bisulfite sequencing because its per-sample cost are comparable to the other methods and realistic for large sample sizes. (iii) The Infinium HumanMethylation assay is useful in the methods, systems and assays as disclosed herein because of its wide use and easy integration with existing genotyping pipelines; and is also a microarray-based method. In some embodiments, other DNA methylation methods that utilize microarrays and or Methylation-specific digestion can be used in the methods, systems and assays as disclosed herein, as these have been benchmarked previously. The methods for performing these assays and the analysis of the date is disclosed herein in the Examples, in the Methods section under the subtitle “Other DNA methylation mapping methods”.

A large number of different epigenetic profiling technologies have been developed (e.g., Laird, P. W. Hum MoI Genet. 14, R65-R76, 2005; Laird, P. W. Nat Rev Cancer 3, 253-66, 2003; Squazzo, S. L. et al. Genome Res 16, 890-900, 2006; and Lieb, J. D. et al. Cytogenet Genome Res 114, 1-15, 2006, all incorporated by reference herein). These can be divided broadly into chromatin interrogation techniques, which rely primarily on chromatin immunoprecipitation with antibodies directed against specific chromatin components or histone modifications, and DNA methylation analysis techniques. Chromatin immunoprecipitation can be combined with hybridization to high-density genome tiling microarrays (ChIP-Chip) to obtain comprehensive genomic data. However, chromatin immunoprecipitation is not able to detect epigenetic abnormalities in a small percentage of cells, whereas DNA methylation analysis has been successfully applied to the highly sensitive detection of tumor-derived free DNA in the bloodstream of cancer patients (Laird, P. W. Nat Rev Cancer 3, 253-66, 2003). Preferably, a sensitive, accurate, fluorescence-based methylation-specific PCR assay (e.g., METHYLIGHT™) is used, which can detect abnormally methylated molecules in a 10,000-fold excess of unmethylated molecules (Eads, C A. et al., Nucleic Acids Res 28, E32, 2000), or an even more sensitive variation of METHYLIGHT™ that allows detection of a single abnormally methylated DNA molecule in a very large volume or excess of unmethylated molecules. In particular aspects, METHYLIGHT™ analyses are performed as previously described by the present applicants {e.g., Weisenberger, D J. et al. Nat Genet. 38:787-793, 2006; Weisenberger et al., Nucleic Acids Res 33:6823-6836, 2005; Siegmund et al., Bioinformatics 25, 25, 2004; Eads et al., Nucleic Acids Res 28, E32, 2000; Virmani et al., Cancer Epidemiol Biomarkers Prey 11:291-297, 2002; Uhlmann et al., Int J Cancer 106:52-9, 2003; Ehrlich et al., Oncogene 25:2636-2645, 2006; Eads et al., Cancer Res 61:3410-3418, 2001; Ehrlich et al., Oncogene 21; 6694-6702, 2002; Marjoram et al., BMC Bioinformatics 7, 361, 2006; Eads et al., Cancer Res 60:5021-5026, 2000; Marchevsky et al., /Mol Diagn 6:28-36, 2004; Sarter et al., Hum Genet. 117:402-403, 2005; Trinh et al., Methods 25:456-462, 2001; Ogino et al., Gut 55:1000-1006, 2006; Ogino et al., J Mol Diagn 8:209-217, 2006, and Woodson, K. et al. Cancer Epidemiol Biomarkers Prey 14:1219-1223, 2005).

High-throughput Illumina platforms, for example, can be used to screen PRC2 targets (or other targets) for aberrant DNA methylation in a large collection of human ES cell DNA samples (or other derivative and/or precursor cell populations), and then METHYLIGHT™ and METHYLIGHT™ variations can be used to sensitively detect abnormal DNA methylation at a limited number of loci {e.g., in a particular number of cell lines during cell culture and differentiation).

Illumina DNA Methylation Profiling. Illumina, Inc. (San Diego) has recently developed a flexible DNA methylation analysis technology based on their GOLDENGATE™ platform, which can interrogate 1,536 different loci for 96 different samples on a single plate (Bibikova, M. et al. Genome Res 16:383-393, 2006). Recently, Illumina reported that this platform can be used to identify unique epigenetic signatures in human embryonic stem cells (Bibikova, M. et al. Genome Res 16:1075-83, 200)). Therefore, Illumina analysis platforms are preferably used. High-throughput Illumina platforms, for example, can be used to screen PRC2 targets (or other targets) for aberrant DNA methylation in a large collection of human ES cell DNA samples (or other derivative and/or precursor cell populations), and then MethyLight and MethyLight variations can be used to sensitively detect abnormal DNA methylation at a limited number of loci {e.g., in a particular number of cell lines during cell culture and differentiation).

There is extensive experience in the analysis and clustering of DNA methylation data, and in DNA methylation marker selection that can be preferably used (e.g., Weisenberger, D J. et al. Nat Genet. 38:787-793, 2006; Siegmund et al., Bioinformatics 25, 25, 2004; Virmani et al. Cancer Epidemiol Biomarkers Prev 11:291-297, 2002; Marjoram et al., Bioinformatics 7, 361, 2006); Siegmund et al., Cancer Epidemiol Biomarkers Prey 15: 567-572, 2006); and Siegmun & Laird, Methods 27:170-178, 2002, all incorporated herein by reference). For example, stepwise strategies {e.g., Weisenberger et al., Nat Genet. 38:787-793, 2006, incorporated herein) are used as taught by the methods exemplified herein to provide DNA methylation markers that are targets for oncogenic epigenetic silencing in ES cells.

By way of example only, a methylation assay can be conducted by a service provider, e.g. epigenomics (Berlin) and other service providers. Briefly, after quality control was performed on the samples, genomic DNA is treated with sodium bisulphite. PCR primers were designed for the regions of interest in the specified genes. The selected genes of interest, e.g., DNA methylation target genes, such as those listed in Table 12A and/or Table 12C are assessed. For example, if one DNA methylation target gene to be assessed is POU5F1 (annotated OCT4 orthologous human gene) and NANOG genes: POU5F1 gene (reference sequence: NM.sub.-002701) AMP1000122 located at the 59 UTR of the annotated Ensembl transcript POUF1_HUMAN (ENST00000259915), 150 bp upstream of the TSS. NANOG gene (reference sequence: NM.sub.-024865) AMP1000123 located at the 59 UTR of the annotated Ensembl transcript NANOG_HUMAN (ENST00000229307), 25 by upstream of the TSS. In some embodiments, The following bisulphite primers can be used for PCR and for sequencing:

POU5F1
5′-ATGGTGTTTGTGGAAGGGG-AA-3′
and
5′-TCCAAACAACTAAAATATACAAAACCT-3′;
NANOG
(SEQ ID NO: 79)
5′-TAATATGAGGTAATTAGTTTAGTTTAGT-3′
and
(SEQ ID NO: 80)
5′-TAATTTCAAACTCTAACTTCAAATAAT-3′.

Other methods are known in the art for determining methylation status of a promoter region, including, but not limited to, array-based methylation analysis and Southern blot analysis.

Methods using an amplification reaction, for example methods above for detecting hypomethylation or hypermethylation of one or more promoter regions, can utilize a real-time detection amplification procedure. For example, the method can utilize molecular beacon technology (Tyagi et al., Nature Biotechnology, 14: 303 (1996)) or Taqman™ technology (Holland et al., Proc. Natl. Acad. Sci. USA, 88:7276 (1991)).

Also methyl light (Trinh et al., Methods 25(4):456-62 (2001), incorporated herein in its entirety by reference), Methyl Heavy (Epigenomics, Berlin, Germany), or SNuPE (single nucleotide primer extension) (see e.g., Watson et al., Genet Res. 75(3):269-74 (2000)) can be used in the methods of the present invention related to identifying altered methylation of gene promoter regions.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, for example, whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art {see, e.g., Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

The degree of methylation in the DNA associated with the promoter region being assessed, may be measured by fluorescent in situ hybridization (FISH) by means of probes which identify and differentiate between genomic DNAs, associated with the DMRs being assessed, which exhibit different degrees of DNA methylation. FISH is described, for example, in de Capoa et al. (Cytometry. 31:85-92, 1998) which is incorporated herein by reference. In this case, the biological sample will typically be any which contains sufficient whole cells or nuclei to perform short term culture. Usually, the sample will be a sample that contains 10 to 10,000, or, for example, 100 to 10,000, whole cells.

Additionally, as mentioned above, methyl light, methyl heavy, and array-based methylation analysis can be performed, by using bisulfite treated DNA that is then PCR-amplified, against microarrays of oligonucleotide target sequences with the various forms corresponding to unmethylated and methylated DNA.

Gene Expression Assays

In some embodiments, gene expression is determined on any gene level, for example, the expression of non-coding genes, as well as non-coding transcripts e.g., natural antisense transcripts (NATs), microRNA (miRNAs) genes and all other types of nucleic acid and/or RNA transcripts that are normally or abnormally present in pluripotent and differentiated cells.

In some embodiments, where the level of gene expression measured is the level of gene transcript expression measured, protein expression gene transcript expression can be measured at the level of messenger RNA (mRNA). In some embodiments, detection uses nucleic acid or nucleic acid analogues, for example, but not limited to, nucleic acid analogous comprise DNA, RNA, PNA, pseudo-complementary DNA (pcDNA), locked nucleic acid and variants and homologues thereof. In some embodiments, gene transcript expression can be assessed by reverse-transcription polymerase-chain reaction (RT-PCR) or quantitative RT-PCR by methods commonly known by persons of ordinary skill in the art.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, a gene expression target gene can be determined by reverse-transcription (RT) PCR and by quantitative RT-PCR (QRT-PCR) or real-time PCR methods. Methods of RT-PCR and QRT-PCR are well known in the art, and are described in more detail below.

Real time PCR is an amplification technique that can be used to determine levels of mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-106 copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan° probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq®, results in the cleavage of the TaqMan probe. This cleavage separates the 5′ fluorescent dye and the 3′ quenching agent, thereby resulting in an increase in fluorescence as a function of amplification (see, for example, at the world-wide web site: “perkin-elmer-dot-com”).

In another embodiment, detection of RNA transcripts can be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et. al. Hepatology, 37(5): 1056-1066, 2003, which is herein incorporated by reference in its entirety.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. In such an embodiment, probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the present invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

Oligonucleotides corresponding to gene expression target gene are immobilized on a chip which is then hybridized with labeled nucleic acids of a test sample obtained from a patient. A positive hybridization signal is obtained with a sample containing a gene expression target gene mRNA transcript. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

Microarrays

A microarray is an array of discrete regions, typically nucleic acids, which are separate from one another and are typically arrayed at a density of between, about 100/cm.sup.2 to 1000/cm.sup.2, but can be arrayed at greater densities such as 10000/cm.sup.2. The principle of a microarray experiment, is that mRNA from a given cell line or tissue is used to generate a labeled sample typically labeled cDNA, termed the ‘target’, which is hybridized in parallel to a large number of, nucleic acid sequences, typically DNA sequences, immobilized on a solid surface in an ordered array.

Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 μm in size and are spaced about the same distance apart. Using this technique, arrays consisting of more than 30,000 cDNAs can be fitted onto the surface of a conventional microscope slide. For oligonucleotide arrays, short 20-25 mers are synthesized in situ, either by photolithography onto silicon wafers (high-density-oligonucleotide arrays from Affymetrix or by ink-jet technology (developed by Rosetta Inpharmatics, and licensed to Agilent Technologies).

Alternatively, presynthesized oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of presynthesized longer oligonucleotides (50-100 mers) has recently been developed to counteract these disadvantages.

Thus in performing a microarray to ascertain the level of gene expression of target gene expression genes in pluripotent stem cells, the following steps can be performed: obtain mRNA from the sample comprising pluripotent stem cells and prepare nucleic acids targets, contact the array under conditions, typically as suggested by the manufactures of the microarray (suitably stringent hybridization conditions such as 3×SSC, 0.1% SDS, at 50 degrees C.) to bind corresponding probes on the array, wash if necessary to remove unbound nucleic acid targets and analyze the results.

It will be appreciated that the mRNA may be enriched for sequences of interest such as those present in a gene profile as described herein by methods known in the art, such as primer specific cDNA synthesis. The population may be further amplified, for example, by using PCR technology. The targets or probes are labeled to permit detection of the hybridization of the target molecule to the microarray. Suitable labels include isotopic or fluorescent labels which can be incorporated into the probe.

The Affymetrix HG-U133.Plus 2.0 gene chips can be used and hybridized, washed and scanned according to the standard Affymetrix protocols. Some RNAs can be replicated on arrays, making 96 the total number of available hybridizations for subsequent analysis.

To monitor mRNA levels, for example, mRNA is extracted from the sample comprising pluripotent stem cells to be tested, reverse transcribed, and fluorescent-labeled cDNA probes are generated. The microarrays capable of hybridizing to gene expression target cDNA's are then probed with the labeled cDNA probes, the slides scanned and fluorescence intensity measured. This intensity correlates with the hybridization intensity and expression levels.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided, for example, in Innis et al. (1990) PCR Protocols, A Guide to Methods and Applications, Academic Press, Inc. N.Y.

Although the same procedures and hardware described by Affymetrix could be employed in connection with the present invention, other alternatives are also available. Many reviews have been written detailing methods for making microarrays and for carrying out assays (see, e.g., Bowtell, Nature Genetics Suppl. 27:25-32 (1999); Constantine, et al, Life ScL News 7:11-13 (1998); Ramsay, Nature Biotechnol. 16:40-44 (1998)). In addition, patents have issued describing techniques for producing microarray plates, slides and related instruments (U.S. Pat. No. 6,902,702; U.S. Pat. No. 6,594,432; U.S. Pat. No. 5,622,826, which are incorporated herein in their entirety by reference) and for carrying out assays (U.S. Pat. No. 6,902,900; U.S. Pat. No. 6,759,197 which are incorporated herein in their entirety by reference). The two main techniques for making plates or slides involve either polylithographic methods (see U.S. Pat. No. 5,445,934; U.S. Pat. No. 5,744,305 which are incorporated herein in their entirety by reference) or robotic spotting methods (U.S. Pat. No. 5,807,522 which are incorporated herein in their entirety by reference). Other procedures may involve inkjet printing or capillary spotting (see, e.g., WO 98/29736 or WO 00/01859 which are incorporated herein in their entirety by reference).

The substrate used for microarray plates or slides can be any material capable of binding to and immobilizing oligonucleotides including plastic, metals such a platinum and glass. A preferred substrate is glass coated with a material that promotes oligonucleotide binding such as polylysine (see Chena, et al, Science 270:467-470 (1995)). Many schemes for covalently attaching oligonucleotides have been described and are suitable for use in connection with the present invention (see, e.g., U.S. Pat. No. 6,594,432 which is incorporated herein in its entirety by reference). The immobilized oligonucleotides should be, at a minimum, 20 bases in length and should have a sequence exactly corresponding to a segment in the gene targeted for hybridization.

### Therapeutic Uses

Various disease and disorders have been suggested as potential targets for stem cell therapy, such as cancer, diabetes, cardiac failure, muscle damage, Celiac Disease, neurological disorder, neurodegenerative disorder, and lysosomal storage diseases, as well as, any of the following diseases, ALS, Parkinson, monogenetic diseases and Mendelian diseases, ageing, general wear and tear of the human body, rheumatic arthritis and other inflammatory diseases, birth defects, etc. Accordingly, the assays, methods, systems and kits of the invention can be used to select pluripotent stem cells for administering to a subject for treatment.

Therefore, in one aspect the invention provide for a method of treatment, prevention, or amelioration of disease or disorder in a subject, the method comprising administering to the subject a stable reprogrammed stem cell as disclosed herein, e.g., a population of piPSC or differentiated cells derived from piPSC, wherein the piPSC are selected by an method as disclosed herein. Without limitation, in some embodiments, a population of piPSCs can be treated for differentiation along a specific lineage before administration to a subject. In some embodiments, a population of piPSCs can be modified or treated to prevent rejection by the recipient subject before administration to the subject.

Routes of administration suitable for the methods of the invention include both local and systemic administration. Generally, local administration results in of the cells being delivered to a specific location as compared to the entire body of the subject, whereas, systemic administration results in delivery of the cells to essentially the entire body of the subject. Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. One method of local administration is by intramuscular injection.

One preferred method of administration is transplantation of such a population of piPSCs, or differentiated progeny derived from such piPSC in a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). Skilled artisan is well aware of methods for implanting or transplantation of cells for treatment of various disease, which are amenable to the present invention.

For administration to a subject, a population of piPSCs, or differentiated progeny derived from such piPSC can be provided in pharmaceutically acceptable compositions. These pharmaceutically acceptable compositions comprise one or more of the pluripotent cells, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, cells can be implanted into a subject or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In the context of administering stable reprogrammed stem cell (piPSC) as disclosed herein, the term “administering” also includes transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species).

In some embodiments, a population of piPSCs, or differentiated progeny derived from such piPSC can be administrated to a subject in combination with a pharmaceutically active agent. As used herein, the term “pharmaceutically active agent” refers to an agent which, when released in vivo, possesses the desired biological activity, for example, therapeutic, diagnostic and/or prophylactic properties in vivo. It is understood that the term includes stabilized and/or extended release-formulated pharmaceutically active agents. Exemplary pharmaceutically active agents include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with autoimmune disease or inflammation. In addition, the methods and compositions described herein can be used to treat domesticated animals and/or pets.

A subject can be one who has been previously diagnosed with or identified as suffering from or having a disorder characterized with a disease for which a stem cell based therapy would be useful.

A subject can be one who is not currently being treated with a stem cell based therapy.

In some embodiments of the aspects described herein, the method further comprising selecting a subject with a disease that would benefit from a stem cell based therapy.

As used herein, the term “neurodegenerative disease or disorder” comprises a disease or a state characterized by a central nervous system (CNS) degeneration or alteration, especially at the level of the neurons such as Alzheimer's disease, Parkinson's disease, Huntington's disease, amyotrophic lateral sclerosis, epilepsy and muscular dystrophy. It further comprises neuro-inflammatory and demyelinating states or diseases such as leukoencephalopathies, and leukodystrophies. Exemplary, neurodegenerative disorders include, but are not limited to, AIDS dementia complex, Adrenoleukodystrophy, Alexander disease, Alpers' disease, Alzheimer's disease, Amyotrophic lateral sclerosis, Ataxia telangiectasia, Batten disease, Bovine spongiform encephalopathy, Canavan disease, Corticobasal degeneration, Creutzfeldt-Jakob disease, Dementia with Lewy bodies, Fatal familial insomnia, Frontotemporal lobar degeneration, Huntington's disease, Infantile Refsum disease, Kennedy's disease, Krabbe disease, Lyme disease, Machado-Joseph disease, Multiple sclerosis, Multiple system atrophy, Neuroacanthocytosis, Niemann-Pick disease, Parkinson's disease, Pick's disease, Primary lateral sclerosis, Progressive supranuclear palsy, Refsum disease, Sandhoff disease, Diffuse myelinoclastic sclerosis, Spinocerebellar ataxia, Subacute combined degeneration of spinal cord, Tabes dorsalis, Tay-Sachs disease, Toxic encephalopathy, and Transmissible spongiform encephalopathy.

As used herein, the term “cancer” includes a malignancy characterized by deregulated or uncontrolled cell growth, for instance carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. An “adenocarcinoma” refers to a carcinoma derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the pluripotent cells selected by the assays, kits, methods, and systems of the invention can be used to treat many kinds of cancers, such as oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kidney chromophobic carcinoma, kidney clear cell carcinoma, kidney oncocytoma, kidney papillary carcinoma, testis non-seminomatous cancer, testis seminoma, urinary bladder transitional carcinoma, lung adenocarcinoma, lung large cell cancer, lung small cell cancer, lung squamous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma abnormal cell growth.

### Drug Screening

Recently, numerous publications have recently highlighted problems with fully reprogrammed iPSCs linked to complete reprogramming or perhaps independent of the particular method used (see Lister et al., Hotspots of aberrant epigenomic reprogramming in human induced pluripotent stem cells. Nature 2011, 471:68-73; Gore et al., Somatic coding mutations in human induced pluripotent stem cells. Nature 2011, 471:63-7; Laurent et al., Dynamic changes in the copy number of pluripotency and cell proliferation genes in human ESCs and iPSCs during reprogramming and time in culture. Cell Stem Cell 2011, 8:106-18, and Hussein et al., Copy number variation and selection during reprogramming to pluripotency. Nature 2011, 471:58-62). These include defects related to mutations, gene copy number variation, and incomplete resetting of DNA methylation. Some of these abnormalities may persist in differentiated cells produced from the iPSCs; some may be selected against by repetitive passaging.

Thus, it is likely that a population of partially reprogrammed cells (piPSC) as disclosed herein will have much fewer problems than those associated with the fully reprogrammed cells (e.g., iPSCs), and therefore piPSCs will have significant value in assays and drug discovery, as well as less line-to-line variability, as well as selecting a particular population of piPSCs that preferentially differentiate along a particular cell lineage of interest, and e.g., where the piPSCs is derived from a somatic cell obtained from a subject with particular disease causing mutation and/or SNP and/or genotype, can be used to provide a pathological aspects of a particular disease.

Additionally, it has recently been reported that for directed differentiation of fully reprogrammed iPSCs along specific linages, the iPSCs under go a complex protocol which requires a carefully controlled addition of multiple growth factors for effective and efficient differentiation into the desired cell line (see Katterman et al., Cell Stem Cell, 2011, 8; 228-40). Thus, piPSC would be advantageous than using iPSCs, as one can select a piPSC cell population which has a predisposition to differentiate along a particular lineage, thus overriding the variability which occurs in the directed differentiation of iPSC using a complicated combination of growth factors.

In spite of this significant concern, iPSCs may still have significant value in drug discovery. However, in that context, there is another potential problem. iPSC clones, even those prepared from a single patient, vary in their capacity to give rise to differentiated cells. Such variability has been seen previously with human ESC lines [24], which can show significant differences although they all meet the standard criteria for ESCs. That is, although they were all able to give rise to cells from the three germ layers in vitro and form teratomas in mice, some gave rise to endodermal lineages well, some gave rise to mesodermal lineages well, and so on. Thus, the standard criteria used to define pluripotency do not preclude line-to-line variability.

In some embodiments, the isolate stable reprogrammed stem cell (piPSC) as disclosed herein can be used in methods, assays, systems and kits to develop specific in vitro assays using isolated stable reprogrammed stem cell (piPSC) as disclosed herein. Existing assays for drug screening/testing and toxicology studies have several shortcomings because they are of animal origin, immortalized cell lines, or derived from cadavers. Because these alternatives often poorly reflect the physiology of normal human cells, stem-cell derived assays (e.g., homogeneous populations of heart and liver cells) could be established in the future and may play an important role for these purposes. For example, the methods, assays, systems, and kits of the invention can be used to identify and isolate stable reprogrammed stem cell (piPSC) as disclosed herein that can differentiate along a lineage which is phenotypic of a disease. In addition to, or alternatively, the methods, assays, systems, and kits of the invention can be used to identify and/or isolate stable reprogrammed stem cell (piPSC) as disclosed herein that can differentiate into an organ, and/or tissue lineage, or a part thereof. Such identified stable reprogrammed stem cell then can be used for screening a test compound.

Furthermore, the flurry of new information now available on the molecular and cellular level related to human diseases (e.g., microarray data) makes it crucial to develop and test hypotheses about pathogenetic interrelations. The experimental access to specific cell types from all developmental stages and even from blastocysts deemed to harbor pathology based on pre-implantation genetic diagnosis may be useful in modeling and understanding aspects of human disease. Thus, such cell lines would also be valuable for the testing of drugs.

Accordingly, the invention provides a method for screening a test compound for biological activity, the method comprising: (a) obtaining a isolated stable reprogrammed stem cell (piPSC) as disclosed herein; (b) optionally causing or permitting the stable reprogrammed stem cell to differentiate to the specific lineage; (c) contacting the cell with a test compound; and (d) determining any effect of the compound on the cell. The effect on the cell can be one that is directly observable or indirectly by use of reporter molecules.

As used herein, the term “biological activity” or “bioactivity” refers to the ability of a test compound to affect a biological sample. Biological activity can include, without limitation, elicitation of a stimulatory, inhibitory, regulatory, toxic or lethal response in a biological assay. For example, a biological activity can refer to the ability of a compound to modulate the effect of an enzyme, block a receptor, stimulate a receptor, modulate the expression level of one or more genes, modulate cell proliferation, modulate cell division, modulate cell morphology, or a combination thereof. In some instances, a biological activity can refer to the ability of a test compound to produce a toxic effect in a biological sample.

As discussed above, the specific lineage can be a lineage which is phenotypic and/or genotypic of a disease. Alternatively, the specific lineage can be lineage which is phenotypic and/or genotypic of an organ and/or tissue or a part thereof.

As used herein, the term “test compound” refers to the collection of compounds that are to be screened for their ability to have an effect on the cell. Test compounds may include a wide variety of different compounds, including chemical compounds, mixtures of chemical compounds, e.g., polysaccharides, small organic or inorganic molecules (e.g. molecules having a molecular weight less than 2000 Daltons, less than 1000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the test compounds may be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds may be screened individually, or in groups. Group screening is particularly useful where hit rates for effective test compounds are expected to be low such that one would not expect more than one positive result for a given group.

A number of small molecule libraries are known in the art and commercially available. These small molecule libraries can be screened for inflammasome inhibition using the screening methods described herein. For example, libraries from Vitas-M Lab and Biomol International, Inc. Chemical compound libraries such as those from of 10,000 compounds and 86,000 compounds from NIH Roadmap, Molecular Libraries Screening Centers Network (MLSCN) can be screened. A comprehensive list of compound libraries can be found at http://www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Without limitation, the compounds can be tested at any concentration that can exert an effect on the cells relative to a control over an appropriate time period. In some embodiments, compounds are testes at concentration in the range of about 0.01 nM to about 1000 mM, about 0.1 nM to about 500 μM, about 0.1 μM to about 20 μM, about 0.1 μM to about 10 μM, or about 0.1 μM to about 5 μM.

The compound screening assay may be used in a high through-put screen. High through-put screening is a process in which libraries of compounds are tested for a given activity. High through-put screening seeks to screen large numbers of compounds rapidly and in parallel. For example, using microtiter plates and automated assay equipment, a pharmaceutical company may perform as many as 100,000 assays per day in parallel.

The compound screening assays of the invention may involve more than one measurement of the observable reporter function. Multiple measurements may allow for following the biological activity over incubation time with the test compound. In one embodiment, the reporter function is measured at a plurality of times to allow monitoring of the effects of the test compound at different incubation times.

The screening assay may be followed by a subsequent assay to further identify whether the identified test compound has properties desirable for the intended use. For example, the screening assay may be followed by a second assay selected from the group consisting of measurement of any of: bioavailability, toxicity, or pharmacokinetics, but is not limited to these methods.

In Some Embodiments of the Present Invention May be Defined in any of the Following Numbered Paragraphs:
1. An isolated reprogrammed cell induced from a differentiated cell, wherein the reprogrammed cell has been reprogrammed to a less differentiated state than the differentiated cell and is capable of being further reprogrammed to a less differentiated state, and wherein the reprogrammed cell can form colonies with distinct morphologies and is capable of self renewing for at least twenty passages before senescence.
2. The reprogrammed cell of paragraph 1, wherein the cell can differentiate into all three primary germ layer lineages selected from; endoderm lineage, mesoderm lineage and ectoderm lineage.
3. The reprogrammed cell of paragraph 1, wherein the reprogrammed cell has a doubling time of less than 15 hours.
4. The reprogrammed cell of paragraph 3, wherein the reprogrammed cell has a doubling time of between 15 and 5 hours.
5. The reprogrammed cell of paragraph 3, wherein the reprogrammed cell has a significantly faster doubling time as compared to an induced pluripotent stem (iPS) cell.
6. The reprogrammed cell of paragraph 5, wherein the reprogrammed cell has a doubling time of at least 1.5 fold faster as compared to an induced pluripotent stem (iPS) cell.
7. The reprogrammed cell of paragraph 5, wherein the reprogrammed cell has a doubling time of at least about 2-fold faster as compared to an induced pluripotent stem (iPS) cell.
8. The reprogrammed cell of paragraph 1, wherein the cell has a lower expression of Dnmt3b by a statistically significant amount relative to the level of expression of an induced pluripotent stem (iPS) cell.
9. The reprogrammed cell of paragraph 8, wherein the reprogrammed cell has at least a 100-fold lower expression of Dnm3b as compared to the level of expression of an induced pluripotent stem (iPS) cell.
10. The reprogrammed cell of paragraph 8, wherein the reprogrammed cell has at least a 200-fold lower expression of Dnm3b as compared to the level of expression of an induced pluripotent stem (iPS) cell.
11. The reprogrammed cell of paragraph 8, wherein the reprogrammed cell has at least a 500-fold lower expression of Dnm3b as compared to the level of expression of an induced pluripotent stem (iPS) cell.
12. The reprogrammed cell of any of paragraphs 1-11, wherein the reprogrammed cell has a lower expression of at least 2 of the following genes; endogenous Oct4, endogenous Nanog, endogenous Rex1, endogenous Tdgf1 by a statistically significant amount relative to the level of expression of an induced pluripotent stem (iPS) cell.
13. The reprogrammed cell of paragraph 12, wherein the reprogrammed cell has at least a 100-fold lower expression of endogenous Nanog as compared to the level of expression of an induced pluripotent stem (iPS) cell.
14. The reprogrammed cell of paragraph 12, wherein the reprogrammed cell has at least a 1000-fold lower expression of endogenous Nanog as compared to the level of expression of an induced pluripotent stem (iPS) cell.
15. The reprogrammed cell of paragraph 12, wherein the reprogrammed cell has at least a 5000-fold lower expression of endogenous Nanog as compared to the level of expression of an induced pluripotent stem (iPS) cell.
16. The reprogrammed cell of paragraph 12, wherein the reprogrammed cell has about a 10,000-fold lower expression of endogenous Nanog as compared to the level of expression of an induced pluripotent stem (iPS) cell.
17. The reprogrammed cell of any of paragraphs 1-16, wherein the reprogrammed cell has a lower expression of endogenous Lefty 2 by a statistically significant amount relative to the level of expression of an induced pluripotent stem (iPS) cell.
18. The reprogrammed cell of paragraph 17, wherein the reprogrammed cell has at least a 100-fold lower expression of endogenous Lefty 2 as compared to the level of expression of an induced pluripotent stem (iPS) cell.
19. The reprogrammed cell of paragraph 17, wherein the reprogrammed cell has at least a 1000-fold lower expression of endogenous Lefty 2 as compared to the level of expression of an induced pluripotent stem (iPS) cell.
20. The reprogrammed cell of paragraph 17, wherein the reprogrammed cell has at least a 10,000-fold lower expression of endogenous Lefty 2 as compared to the level of expression of an induced pluripotent stem (iPS) cell.
21. The reprogrammed cell of paragraph 17, wherein the reprogrammed cell has about between a 15,000-fold and 10,000-fold lower expression of endogenous Lefty 2 as compared to the level of expression of an induced pluripotent stem (iPS) cell.
22. The reprogrammed cell of any of paragraphs 1-21, wherein the reprogrammed cell has a higher level of expression of any one of endogenous TGDF1, endogenous Tert or endogenous Sox2 by a statistically significant amount relative to the level of expression of a somatic cell.
23. The reprogrammed cell of paragraph 22, wherein the reprogrammed cell has at least a 1,000-fold higher expression of endogenous Tgdf1 as compared to the level of expression of endogenous Tgdf1 in a somatic cell.
24. The reprogrammed cell of paragraph 22, wherein the reprogrammed cell has at least a 5,000-fold higher expression of endogenous Tgdf1 as compared to the level of expression of endogenous Tgdf1 in a somatic cell.
25. The reprogrammed cell of paragraph 22, wherein the reprogrammed cell has at least a 7,000-fold higher expression of endogenous Tgdf1 as compared to the level of expression of endogenous Tgdf1 in a somatic cell.
26. The reprogrammed cell of paragraph 22, wherein the reprogrammed cell has about a 7,000-fold to 15,000-fold higher expression of endogenous Tgdf1 as compared to the level of expression of endogenous Tgdf1 in a somatic cell.
27. The reprogrammed cell of paragraph 22, wherein the reprogrammed cell has at least a 5-fold higher expression of endogenous Tert as compared to the level of expression of endogenous Tert in a somatic cell.
28. The reprogrammed cell of paragraph 22, wherein the reprogrammed cell has at least a 7-fold higher expression of endogenous Tert as compared to the level of expression of endogenous Tert in a somatic cell.
29. The reprogrammed cell of paragraph 22, wherein the reprogrammed cell has between a 7-fold and 20-fold higher expression of endogenous Tert as compared to the level of expression of endogenous Tert in a somatic cell.
30. The reprogrammed cell of paragraph 22, wherein the reprogrammed cell has at least a 100-fold higher expression of endogenous Sox2 as compared to the level of expression of endogenous Sox2 in a somatic cell.
31. The reprogrammed cell of paragraph 22, wherein the reprogrammed cell has at least a 500-fold higher expression of endogenous Sox2 as compared to the level of expression of endogenous Sox2 in a somatic cell.
32. The reprogrammed cell of paragraph 22, wherein the reprogrammed cell has between a 500-fold and 100,000-fold higher expression of endogenous Sox2 as compared to the level of expression of endogenous Sox2 in a somatic cell.
33. The reprogrammed cell of paragraph 32, wherein the reprogrammed cell has between a 500-fold and 10,000-fold higher expression of endogenous Sox2 as compared to the level of expression of endogenous Sox2 in a somatic cell.
34. The reprogrammed cell of paragraph 1, wherein the reprogrammed cell has a lower expression of Krt10 or Thy1 or Krt10 and Thy1 by a statistically significant amount relative to the level of expression of the isogenic cell from which the reprogrammed cell was derived.
35. The reprogrammed cell of paragraph 1, wherein the reprogrammed cell has an increased CpG methylation of Oct4 or Nanog or Oct4 and Nanog a statistically significant amount relative to the level of CpG methylation of Oct4 or Nanog or Oct4 and Nanog an induced pluripotent stem (iPS), and/or a decreased CpG methylation of Oct4 or Nanog or Oct4 and Nanog a statistically significant amount relative to the level of CpG methylation of Oct4 or Nanog or Oct4 and Nanog an the isogenic cell from which the reprogrammed cell was derived.
36. The reprogrammed cell of paragraph 1, wherein the reprogrammed cell has an increased rate of proliferation by a statistically significant amount relative to the level of expression of an induced pluripotent stem (iPS) cell.
37. The reprogrammed cell of paragraph 1, wherein the reprogrammed cell has an increased expression of Xist in female lines by a statistically significant amount to the level of expression of the somatic cell from which the reprogrammed cell was derived.
38. The reprogrammed cell of paragraph 37, wherein the reprogrammed cell has at least a 5-fold higher expression of Xist in female lines as compared to the level of expression of endogenous Xist in a female induced pluripotent stem (iPS) cell.
39. The reprogrammed cell of paragraph 37, wherein the reprogrammed cell has between a 5-fold and a 50-fold higher expression of Xist in female lines as compared to the level of expression of endogenous Xist in a female induced pluripotent stem (iPS) cell.
40. The reprogrammed cell of paragraph 37, wherein the reprogrammed cell has between a 5-fold and a 20-fold higher expression of Xist in female lines as compared to the level of expression of endogenous Xist in a female induced pluripotent stem (iPS) cell.
41. The reprogrammed cell of paragraph 1, wherein the reprogrammed cell is not an induced pluripotent stem (iPS) cell.
42. The reprogrammed cell of any of paragraphs 1 to 41, wherein the reprogrammed cell has not undergone complete remodeling of the epigenome.
43. The reprogrammed cell of any of paragraphs 1 to 42, wherein the reprogrammed cell has undergone a significantly less amount of remodeling of the epigenome as compared to an induced pluripotent stem (iPS) cell.
44. The reprogrammed cell of paragraph 37, wherein the reprogrammed cell has undergone at least 10% less remodeling of the epigenome as compared to a female induced pluripotent stem (iPS) cell.
45. The reprogrammed cell of paragraph 37, wherein the reprogrammed cell has undergone at least 20% less remodeling of the epigenome as compared to a female induced pluripotent stem (iPS) cell.
46. The reprogrammed cell of paragraph 37, wherein the reprogrammed cell has undergone at least 30% less remodeling of the epigenome as compared to a female induced pluripotent stem (iPS) cell.
47. The reprogrammed cell of paragraph 37, wherein the reprogrammed cell has undergone between 10% and 50% less remodeling of the epigenome as compared to a female induced pluripotent stem (iPS) cell.
48. The reprogrammed cell of any of paragraphs 1 to 47, wherein the reprogrammed cell can be further reprogrammed into an induced pluripotent stem (iPS) cell.
49. The reprogrammed cell of any of paragraphs 1 to 48, wherein the reprogrammed cell is a multipotent cell.
50. The reprogrammed cell of any of paragraphs 1 to 49, wherein the reprogrammed cell has at least a 10-fold lower expression of Dnmt3b relative to the level of expression of an induced pluripotent stem (iPS) cell.
51. The reprogrammed cell of any of paragraphs 1 to 50, wherein the reprogrammed cell has at least a 15-fold lower expression of Dnmt3b relative to the level of expression of an induced pluripotent stem (iPS) cell.
52. The reprogrammed cell of any of paragraphs 1 to 51, wherein the reprogrammed cell is capable of multilineage differentiation.
53. The reprogrammed cell of any of paragraphs 1 to 52, wherein the efficiency of differentiation along different lineages between clones.
54. The reprogrammed cell of paragraph 53, wherein some reprogrammed cells differentiate along neuronal lineages with greater efficiency that other reprogrammed cells.
55. The reprogrammed cell of any of paragraphs 1 to 52, wherein the reprogrammed cell is capable of differentiation along neuronal lineages.
56. The reprogrammed cell of any of paragraphs 1 to 52, wherein the reprogrammed cell is capable of differentiation along neuronal lineages at a significantly higher efficiency than that of an induced pluripotent stem (iPS) cell.
57. The reprogrammed cell of any of paragraphs 1 to 56, wherein the reprogrammed cell is can be further reprogrammed to a less differentiated cell by contacting with a small molecule reprogramming factor.
58. The reprogrammed cell of any of paragraphs 1 to 57, wherein the small molecule reprogramming factor is selected from the group consisting of: RepSox, 2i, 5′Ara C or any combination thereof.
59. The reprogrammed cell of any of paragraphs 1 to 58, wherein the reprogrammed cell which can be further reprogrammed to a less differentiated cell has a higher level of H3K4me2 methylation of the Nanog proximal promoter by a statistically significant amount as compared to H3K27me2 methylation of the Nanog proximal promoter.
60. The reprogrammed cell of any of paragraphs 1 to 59, wherein the reprogrammed cell can be directly differentiated into any one of the following cell types: cardiomyocytes, neurons, motor neurons, neurectodermal precursors, definitive endoderm.
61. The reprogrammed cell of any of paragraphs 1 to 60, wherein the reprogrammed cell is selected from the group of reprogrammed cells consisting of; (i) alkaline phosphatase negative, SSEA1 negative, small granular colonies, (ii) alkaline phosphatase positive, SSEA1 negative, compact colonies, and (iii) alkaline phosphatase positive, SSEA1 positive, compact colonies.
62. The reprogrammed cell of any of paragraphs 1 to 61, wherein the reprogrammed cell is a mammalian cell.
63. The reprogrammed cell of any of paragraphs 1 to 61, wherein the reprogrammed cell is a human cell.
64. A method for producing a reprogrammed cell from a differentiated cell, wherein the reprogrammed cell has been reprogrammed to a less differentiated state than the differentiated cell from which it was derived, and wherein the reprogrammed cell is capable of being further reprogrammed to a less differentiated state, and wherein the reprogrammed cell can form colonies with distinct morphologies and is capable of self renewing for at least twenty passages before senescence, the method comprising;
   i. inducing the expression of at least one transcription factor in a somatic cell, wherein the transcription factor is selected from the group consisting of; Sox2, Oct-4, Klf-4, c-Myc, lin-28 and Nanog; and
   ii. isolating a reprogrammed cell by selection based on at least one or any combination of the of following characteristics:
      i. the reprogrammed cell can differentiate into all three primary germ layer lineages selected from; endoderm lineage, mesoderm lineage and ectoderm lineage;
      ii. the reprogrammed cell has a doubling time of less than 15 hours;
      iii. the reprogrammed cell has at least a 100-fold lower expression of Dnm3b as compared to the level of expression of an induced pluripotent stem (iPS) cell;
      iv. the reprogrammed cell has at least a 100-fold lower expression of endogenous Nanog as compared to the level of expression of an induced pluripotent stem (iPS) cell;
      v. the reprogrammed cell has at least a 100-fold lower expression of endogenous Lefty 2 as compared to the level of expression of an induced pluripotent stem (iPS) cell;
      vi. the reprogrammed cell has at least a 1,000-fold higher expression of endogenous Tgdf1 as compared to the level of expression of endogenous Tgdf1 in a somatic cell;
      vii. the reprogrammed cell has at least a 5-fold higher expression of endogenous Tert as compared to the level of expression of endogenous Tert in a somatic cell.
      viii. the reprogrammed cell has at least a 100-fold higher expression of endogenous Sox2 as compared to the level of expression of endogenous Sox2 in a somatic cell;
      ix. the reprogrammed cell has a lower expression of Krt10 or Thy1 or Krt10 and Thy1 by a statistically significant amount relative to the level of expression of the isogenic cell from which the reprogrammed cell was derived;
      x. the reprogrammed cell has an increased CpG methylation of Oct4 or Nanog or Oct4 and Nanog by a statistically significant amount relative to the level of CpG methylation of Oct4 or Nanog or Oct4 and Nanog an induced pluripotent stem (iPS);
      xi. the reprogrammed cell has an decreased CpG methylation of Oct4 or Nanog or Oct4 and Nanog a statistically significant amount relative to the level of CpG methylation of Oct4 or Nanog or Oct4 and Nanog an the isogenic cell from which the reprogrammed cell was derived; and
      xii. the reprogrammed cell has at least a 5-fold higher expression of Xist in female lines as compared to the level of expression of endogenous Xist in a female induced pluripotent stem (iPS) cell.
65. The method of paragraph 64, wherein the exogenous transcription factor is a nucleic acid encoding at least one of the transcription factors selected from the group consisting of: Sox2, Oct-4, Klf-4, c-Myc, lin-28 and Nanog.
66. The method of paragraph 64, wherein inducing the expression of at least one exogenous transcription factor comprises contacting the isogenic cell with at least one agent which increase the expression exogenous transcription factor in the isogenic cell.
67. The method of paragraph 66, wherein the agent is selected from the group consisting of: RepSox, 2i, 5′ AraC, or any combination thereof.
68. The method of paragraph 64, wherein inducing the expression of at least one exogenous transcription factor comprises introducing a nucleic acid sequence into the isogenic cell which encodes at least one gene encoding a reprogramming factor.
69. The method of paragraph 64, wherein inducing the expression of at least one exogenous transcription factor uses a non-viral method.
70. The method of paragraph 66, wherein the at least one agent is at least one compound selected from the group consisting of;
   a) a TGF-β Receptor Type I inhibitor, wherein the TGF-β Receptor Type I inhibitor substitutes for exogenously Sox2 transcription factor, and wherein exogenous Sox2 transcription factor is not present; (e.g. RepSox(E-616452), or SB43142, or E-616451)
   b) an inhibitor of Src signaling pathway, wherein the inhibitor of Src signaling pathway substitutes for exogenously Sox2 transcription factor, and wherein exogenous Sox2 transcription factor is not present; (e.g. EI-275)
   c) an agonist of the Mek/Erk signaling pathway, wherein agonist of the Mek/Erk signaling pathway substitutes for exogenously Klf-4 transcription factor, and wherein exogenous Klf-4 transcription factor is not present; (e.g. PGJ₂)
   d) an inhibitor of Ca²⁺/calmodulin, wherein the inhibitor of Ca²⁺/calmodulin signaling pathway substitutes for exogenously Klf-4 transcription factor, and wherein exogenous Klf-4 transcription factor is not present; (e.g. HBDA)
   e) an inhibitor of EGF signaling, wherein the inhibitor of EGF signaling pathway substitutes for exogenously Klf-4 transcription factor, and wherein exogenous Klf-4 transcription factor is not present; (e.g. HBDA)
   f) an agonist of ATP-dependent potassium channels, wherein the agonist of ATP-dependent potassium channels substitutes for exogenously Oct-4 transcription factor, and wherein exogenous Oct-4 transcription factor is not present; (e.g. Simomenine)
   g) a sodium channel inhibitor, wherein the inhibitor of sodium channels substitutes for exogenously Oct-4 transcription factor, and wherein exogenous Oct-4 transcription factor is not present; (e.g. Simomenine)
   h) an MAPK agonist, wherein the MAPK agonist substitutes for exogenously Oct-4 transcription factor, and wherein exogenous Oct-4 transcription factor is not present; (e.g. Ropivocaine or Bupivicanine).
71. An isolated heterogeneous population of reprogrammed cells comprising at least two different reprogrammed cell populations of paragraphs 1 to 63.
72. The isolated heterogeneous population of paragraph 71, comprising at least three different reprogrammed cell populations of paragraphs 1 to 63.
73. The isolated heterogeneous population of paragraph 71, wherein the different reprogrammed cell populations are selected from the groups consisting of: (i) alkaline phosphatase negative, SSEA1 negative, small granular colonies, (ii) alkaline phosphatase positive, SSEA1 negative, compact colonies, and (iii) alkaline phosphatase positive, SSEA1 positive, compact colonies.
74. The isolated heterogeneous population of paragraph 71, wherein the population comprises less than 10% of iPS cells.
75. The isolated heterogeneous population of paragraph 71, wherein the population comprises less than 5% of iPS cells.
76. The isolated heterogeneous population of paragraph 71, wherein the population comprises less than 2% of iPS cells.
77. The isolated heterogeneous population of paragraph 71, wherein the population comprises less than 1% of iPS cells.
78. Use of a reprogrammed cell of any of paragraphs 1 to 63 for differentiating into a isogenic cell of endoderm lineage, mesoderm lineage or ectodermal lineage, wherein the reprogrammed cell does not become an induced pluripotent stem (iPS) cell prior to differentiating into a cell of endoderm lineage, mesoderm lineage or ectodermal lineage.
79. Use of a reprogrammed cell of any of paragraphs 1 to 63 for reprogramming further to an less differentiated state to produce an induced pluripotent stem (iPS) cell.
80. The use of a reprogrammed cell of paragraph 79, wherein the reprogrammed cell is cultured in the absence of LIF, or the expression of a reprogramming transcription factor is induced in the reprogrammed cell.
81. The use of paragraph 80, wherein the induction of a reprogramming transcription factor is induced by contacting the cell with an agent or RepSox (E-616452).
82. The use of paragraph 80, wherein reprogramming transcription factor is selected from the group consisting of; Sox2, Oct-4, Klf-4, c-Myc, lin-28 and Nanog.
83. The use of paragraph 79, wherein the reprogrammed cell is selected for further reprogramming to an induced pluripotent stem (iPS) cell, wherein the selected reprogrammed cell has a higher level of H3K4me2 methylation of the Nanog proximal promoter by a statistically significant amount as compared to H3K27me2 methylation of the Nanog proximal promoter.
84. A differentiated cell derived from inducing the differentiation of a reprogrammed cell of paragraph 1 to 63.
85. A method for evaluating the toxicity of an agent comprising contacting a reprogrammed cell with a reprogrammed cell of paragraph 1 to 63 with an agent and evaluating the effect of the agent on at least one of the following characteristics of the reprogrammed cell; multilineage differentiation capacity into all 3 germline cell layers, viability, propagation for at least 20 passages.
86. A method for stem cell therapy comprising;
   i. isolating and collecting a somatic cell from a subject;
   ii. reprogramming the somatic cell to a reprogrammed cell of paragraph 1 to 63;
   iii. inducing differentiation of the reprogrammed cell of step (ii); and
   iv. transplanting the differentiated cell from step (iii) into the subject.
87. A method to isolate a reprogrammed cell of paragraph 1 from a population of cells comprising induced pluripotent stem (iPS) cells and somatic cells, the method comprising;
   i. positively selecting for cells with a statistically significant high level of expression at least one of Tdgf1, Tert, Sox2, Pou5f1;
   ii. selecting the cells obtained in step (i) for cells with a significantly low level of expression of at least one of Dnmt3b, Dnmt3a, Rex1, Left2, or Nanog
wherein the selected cells in step (ii) are an isolated reprogrammed cell.
88. A method to isolate a reprogrammed cell of paragraph 1 from a population of cells comprising induced pluripotent stem cells (iPSCs), the method comprising positively selecting for cells with a significantly low level of expression of at least one of Dnmt3b, Dnmt3a, Rex1, Left2, or Nanog.
89. A method to isolate a reprogrammed cell of paragraph 1 from a population of cells comprising induced pluripotent stem cells (iPSCs), the method comprising negatively selecting for cells with a significantly high level of expression of at least one of Dnmt3b, Dnmt3a, Rex1, Left2, or Nanog, wherein the cells with a significantly high level of expression of at least one of Dnmt3b, Dnmt3a, Rex1, Left2, or Nanog are discarded.
90. The method of paragraph 88, wherein the cells selected in the positive selection step (i) have at least a 1,000-fold higher expression of endogenous Tgdf1 as compared to the cells not selected.
91. The method of paragraph 88, wherein the cells selected in the positive selection step (i) have at least a 5,000-fold higher expression of endogenous Tgdf1 as compared to the cells not selected.
92. The method of paragraph 88, wherein the cells selected in the positive selection step (i) have at least a 7,000-fold higher expression of endogenous Tgdf1 as compared to the cells not selected.
93. The method of paragraph 88, wherein the cells selected in the positive selection step (i) have about a 7,000-fold to 15,000-fold higher expression of endogenous Tgdf1 as compared to the cells not selected.
94. The method of paragraph 88, wherein the cells selected in the positive selection step (i) have at least a 5-fold higher expression of endogenous Tert as compared to the cells not selected.
95. The method of paragraph 88, wherein the cells selected in the positive selection step (i) have at least a 7-fold higher expression of endogenous Tert as compared to the cells not selected.
96. The method of paragraph 88, wherein the cells selected in the positive selection step (i) have between a 7-fold and 20-fold higher expression of endogenous Tert as compared to the cells not selected.
97. The method of paragraph 88, wherein the cells selected in the positive selection step (i) have at least a 100-fold higher expression of endogenous Sox2 as compared to the cells not selected.
98. The method of paragraph 88, wherein the cells selected in the positive selection step (i) have at least a 500-fold higher expression of endogenous Sox2 as compared to the cells not selected.
99. The method of paragraph 88, wherein the cells selected in the positive selection step (i) have between a 500-fold and 100,000-fold higher expression of endogenous Sox2 as compared to the cells not selected.
100. The method of paragraph 88, wherein the cells selected in the positive selection step (i) have between a 500-fold and 10,000-fold higher expression of endogenous Sox2 as compared to the cells not selected.
101. The method of paragraph 88, wherein the cells selected in the selection step (ii) have at least a 100-fold lower expression of Dnm3b as compared to the cells not selected.
102. The method of paragraph 88, wherein the cells selected in the selection step (ii) have at least a 200-fold lower expression of Dnm3b as compared to the cells not selected.
103. The method of paragraph 88, wherein the cells selected in the selection step (ii) have at least a 500-fold lower expression of Dnm3b as compared to the cells not selected.
104. The method of paragraph 88, wherein the cells selected in the selection step (ii) have a lower expression of at least 2 of the following genes; endogenous Oct4, endogenous Nanog, endogenous Rex1, endogenous Tdgf1 by a statistically significant amount relative to the cells not selected.
105. The method of paragraph 88, wherein the cells selected in the selection step (ii) have at least a 100-fold lower expression of endogenous Nanog as compared to the cells not selected.
106. The method of paragraph 88, wherein the cells selected in the selection step (ii) have at least a 1000-fold lower expression of endogenous Nanog as compared to the cells not selected.
107. The method of paragraph 88, wherein the cells selected in the selection step (ii) have at least a 5000-fold lower expression of endogenous Nanog as compared to the cells not selected.
108. The method of paragraph 88, wherein the cells selected in the selection step (ii) have about a 10,000-fold lower expression of endogenous Nanog as compared to the cells not selected.
109. The method of paragraph 88, wherein the cells selected in the selection step (ii) have a lower expression of endogenous Lefty 2 as compared to the cells not selected.
110. The method of paragraph 88, wherein the cells selected in the selection step (ii) have at least a 100-fold lower expression of endogenous Lefty 2 as compared to the cells not selected.
111. The method of paragraph 88, wherein the cells selected in the selection step (ii) have at least a 1000-fold lower expression of endogenous Lefty 2 as compared to the cells not selected.
112. The method of paragraph 88, wherein the cells selected in the selection step (ii) have at least a 10,000-fold lower expression of endogenous Lefty 2 as compared to the cells not selected.
113. The method of paragraph 88, wherein the cells selected in the selection step (ii) have about between a 15,000-fold and 10,000-fold lower expression of endogenous Lefty 2 as compared to the cells not selected.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

Methods

Derivation of MEFs and Cell Culture.

MEFs were derived from E12.5 embryos hemizygous for the Oct4::GFP transgenic allele. Gonads and internal organs were removed before processing the embryos for MEF isolation. MEFs were grown in DMEM media (Gibco) supplemented with 10% FBS and penicillin/streptomycin (Gibco).

Retroviral Infection

Viral transduction of three transcription factors reprograms adult somatic cells to induced pluripotent cells that possess many of the known characteristics of embryonic stem cells including global remodeling of the epigenome. Complete remodeling of the epigenome may be disadvantageous for studying disease phenotypes that are encoded in the epigenetic memory of a cell. In this respect, partially reprogrammed iPS cells may more faithfully retain the epigenetic memory of a diseased state when differentiated to a clinically relevant cell type. Differentiation of epigenetic disease relevant cell types may serve as a valuable resource for disease modeling and therapeutic screening. To this end, the inventors demonstrate whether disease relevant cell types obtained from a partially pluripotent state maintain a stronger imprinting of diseased states than those obtained from bona fide pluripotent cells.

Generation of piPS and iPS Cells.

GFP+P0 colonies were picked manually and incubated in 0.25% trypsin (Gibco) for 20 minutes at room temperature before plating on a feeder layer in mES cell media. This process was repeated until passage 3, at which time colonies were trypsinized and passaged in bulk and maintained on feeders in mES cell media. Antibody staining for Sox2 and Nanog and alkaline phosphatase staining iPS and piPS cells were cultured on irradiated MEF feeders in chamber slides, fixed with 4% PFA and stained with primary antibodies against mSox2 (Santa Cruz, sc-17320), mNanog (CosmoBio, REC-RCAB0002 PF), followed by staining with the appropriate secondary antibodies conjugated to Alexa Fluor 546 (Invitrogen). Nuclei were counterstained with Hoechst33342 (Sigma). iPS cells were assayed for alkaline phosphatase activity using the Vector Red alkaline phosphatase assay kit from Vector Laboratories.

In some embodiments, MEFS were infected with the pMXs vector (Takahashi et al., 2007a). MEFs were infected with two to three pools of viral supernatant during a 72 hr period. The first day that viral supernatant was added was termed “day 1 post-infection.” For quantification, Oct4::GFP negative colonies were isolated by manual selection approximately 30 days post transduction. Oct4::GFP negative colonies were subsequently sub cloned an additional two times to insure clonality. Following sub cloning, piPS cell lines were cultured and passaged following standard mESC protocols. All animal research was performed under the oversight of the Office of Animal Resources at Harvard University.

Immunostaining

piPS cells were grown to 70-80% confluence in 10-cm plates (Falcon) in mES cell medium. To form embryoid bodies, cells were washed once with PBS to eliminate mES cell medium and then incubated with 1 nil of 0.25% trypsin (GIBCO) for 5-10 min at room temperature (21-25 ∞C). Cells were then resuspended in 10 nil of DMEM medium supplemented with 10% fetal bovine serum (Hyclone), penicillin, streptomycin, glutamine (GIBCO) and 2-mercaptoethanol (GIBCO), counted, and plated at a concentration of 200,000 cells per ml in Petri dishes (Falcon). Six days later, embryoid bodies were split from one dish into six well plate coated with 0.1% gelatin and cultured for an additional six days in DMEM+10% FBS. Medium was changed every 3 days. The adhered embryoid bodies were stained with primary antibodies against Sox17(AFP) (RnD Systems), Skeletal Myosin (MF20) (Developmental Studies Hybridoma Bank, MF20), or Beta-III-tubulin (TUJ1) (Sigma, T2200), and visualized by staining with a secondary antibody conjugated to Alexa Fluor 546 (Invitrogen).

TaqMan Gene-Expression Arrays

For comparison to mES, iPS, piPS cells and MEFs, cell were grown to near confluence on 0.1% gelatin and RNA was isolated with Trizol (Invitrogen). To eliminate contaminating genomic DNA, RNA samples were treated with Turbo DNase (Ambion) according to manufactures instructions. Complimentary DNA was reverse transcribed using SuperScript III (Invitrogen). A total of 200 ng of cDNA were amplified per a reaction. Mean Ct values were obtained from four quadriplicate reactions. All samples were normalized to 18s rRNA and then calibrated to expression in MEFs.

Spontaneous Differentiation of iPS Cells In Vitro

iPS cells were grown to 70-80% confluence in 10-cm plates (Falcon) in mES cell medium. To form embryoid bodies, cells were washed once with PBS to eliminate mES cell medium and then incubated with 1 ml of 0.25% trypsin (GIBCO) for 5-10 min at room temperature (21-25° C.). Cells were then resuspended in 10 ml of DMEM medium supplemented with 10% fetal bovine serum (Hyclone), penicillin, streptomycin, glutamine (GIBCO) and 2-mercaptoethanol (GIBCO), counted, and plated at a concentration of 200,000 cells per ml in Petri dishes (Falcon). Six days later, embryoid bodies were split from one dish into six well plate coated with 0.1% gelatin and cultured for an additional six days in DMEM+10% FBS. Medium was changed every 3 days. The adhered embryoid bodies were stained with primary antibodies against Alphafetoprotein (AFP) (Dakocytomation, A0008), Skeletal Myosin (MF20) (Developmental Studies Hybridoma Bank, MF20), or Beta-III-tubulin (TUJ1) (Sigma, T2200), Sox17 (RnD Systems) and visualized by staining with a secondary antibody conjugated to Alexa Fluor 546 (Invitrogen).

Directed Differentiation of iPS Cells into Motor Neurons

iPS and mES (V6.5) cells were differentiated into motor neurons according to methods previously described for mouse ES cells differentiation. The iPS and mES cells were grown to 70-80% confluence in 10-cm plates (Falcon) in mES cell medium. To form embryoid bodies, cells were washed once with PBS to eliminate mES cell medium and then incubated with 1 ml of 0.25% trypsin (GIBCO) for 5-10 min at room temperature (21-25° C.). Cells were then resuspended in 10 ml of DM1 medium (DMEM-F12, GIBCO), 10% knockout serum (GIBCO), penicillin, streptomycin, glutamine (GIBCO) and 2-mercaptoethanol (GIBCO), counted and plated at a concentration of 200,000 cells per ml in Petri dishes (Falcon). Two days later, embryoid bodies were split from one dish into four Petri dishes containing DM1 medium supplemented with RAc (100 nM; stock: 1 mM in DMSO, Sigma) and Shh (300 nM, R&D Systems). Medium was changed after 3-4 d. On day 7, the embryoid bodies were dissociated into single-cell suspensions. The suspensions were pelleted in a 15-ml Falcon tube, washed once with PBS, and incubated in Earle's balanced salt solution with 20 units of papain and 1,000 units of DNase I (Worthington Biochemical) for 30-60 min at 37° C. The mixture was then triturated with a 10-ml pipette and centrifuged for 5 min at 300×g. The resulting cell pellet was washed with PBS and resuspended in F12 medium (F12 medium, GIBCO) with 5% horse serum (GIBCO), B-27 supplement (GIBCO), N2 supplement (GIBCO) with neurotrophic factors (GDNF and BDNF, 10 ng/ml, R&D Systems). The cells were counted and plated on poly-D-lysine/laminin culture slides (BD Biosciences) or on a layer of primary glial cells. 3-5 days later, the cultures were fixed with PFA and stained with primary antibodies against TUJ1 (Sigma, T2200) and HB9 (Developmental Studies Hybridoma Bank, 81.5C10), and visualized by staining with secondary antibodies conjugated to Alexa Fluor 488 and Alexa Fluor 546 (Invitrogen). For counting HB9+ cells, motor neurons were differentiated as above except in embryoid body culture without dissociation and plating. Embryoid bodies were sectioned as above and stained with the TUJ1 and HB9 antibodies along with the Alexa Fluor 488 and Alexa Fluor 546 secondary antibodies. Cultures were counterstained with Hoechst 33342 and HB9+ and total nuclei were counted. Numbers were derived from at least 3 different embryoid bodies per cell line.

The cells were counted and plated on poly-D-lysine/laminin culture slides (BD Biosciences) or on a layer of primary glial cells. 3-5 days later, the cultures were fixed with PFA and stained with primary antibodies against TUJ1 (Sigma, T2200) and HB9 (Developmental Studies Hybridoma Bank, 81.5C10), and visualized by staining with secondary antibodies conjugated to Alexa Fluor 488 and Alexa Fluor 546 (Invitrogen).

Directed Differentiation of iPS Cells into Cardiac Myocytes

The piPS, and iPS and mES cells were grown to 70-80% confluence in 10-cm plates (Falcon) in mES cell medium. To form embryoid bodies, cells were washed once with PBS to eliminate mES cell medium and then incubated with 3 ml of 0.25% trypsin (GIBCO) for 5-10 min at room temperature (21-25° C.). Cells were then resuspended in 10 ml of differentiation DMEM containing 15% fetal bovine serum (Hyclone), penicillin, streptomycin, glutamine (GIBCO) and 1 mM Ascrobic acid. The cells were counted and plated onto a 10 cm non-adherent plate (Falcon) at a concentration of 200,000 cells per ml. Media was replaced with fresh media every three days. Six days post plating embryoid bodies were transferred to 6-well plates coated with 0.1% gelatin and cultured for an additional six days. To determine differentiation efficiency the number of beating regions were counted and normalized to the total number of embryoid bodies. Embryoid bodies were stained with alpha actinin (Sigma).

piPS lines induced to differentiate into beating cardiac myocytes by forming EBs in the presence of 1 mM ascorbic acid for 6 days. Ebs were then adhered onto gelatin coated plates and cultured an additional 6 days. Compared number of beating EBs to total number of EBs. Counter stained with alpha actinin to confirm beating clusters of cells were cardiac myocytes.

Teratoma Production and Analysis.

piPSCs form teratomas containing tissue from the three embryonic germ layers upon injection into nude mice. piPSCs form encapsulated teratomas that are immature and of a lower grade as compared to teratomas formed by iPSC or mESC. A confluent 10 cm dish of piPS and iPS cells was trypsinized, pelleted, resuspended in 0.2 mls of mES media, and injected subcutaneously into a CD1-Nude mouse. 3-4 weeks later, teratomas were harvested, fixed overnight with 4% paraformaldehyde, embedded in paraffin, sectioned, HE stained, and analyzed.

Production of Chimeric Mice.

Female ICR mice were superovulated with PMS and hCG and mated to ICR stud males. 24-hours after hCG injection, zygotes were isolated from vaginally plugged females. After culture in KSOM media for 3 days, the resulting blastocysts were injected with ˜5-10 iPS cells from a C57BL6 background pre-labeled with a lentivirus constitutively expressing the red fluorescent protein tdTomato and transferred into pseudopregnant females. Embryos were either harvested at day E13.5 or allowed to develop to term. Chimeric embryos were visualized on a Leica MZ16FA dissecting microscope using RFP and bright field channels. For 8-cell stage injections, zygotes were developed in vitro to the 8-cell stage, injected with iPS cells, further developed in vitro to the blastocyst stage, and visualized.

Chemical Reprogramming of Stable Intermediate Cell Lines.

piPSC or iPSC were plated on irradiated feeders or 0.1% gelatin, and grown in mES media treated with RepSox (E-616452) (25 μM), AZA (500 μM), or both for 48 hours. For 2i treatment, CHIR99021 (Stemgent) was used at 3 μM and PD0325901 (Stemgent) was used at 1 μM. Oct4::GFP+ colonies were scored 12 days after the beginning of chemical treatment. Treatments were performed in mES media containing FBS unless otherwise noted.

RT-PCR

RNA was harvested with Trizol (Invitrogen) and treated with Turbo DNA-free (Ambion) to Remove genomic DNA contamination. RNA was reverse transcribed using random hexamer primers and superscript III reverse transcriptase (Invitrogen). Primer sequences for endogenous genes were the following: Nanog (5′-CAGGTGTTTGAGGGTAGCTC (SEQ ID NO: 1) and 5′-CGGTTCATCATGGTACAGTC (SEQ ID NO: 2)), Sox2 (5′ TAGAGCTAGACTCCGGGCGA TGA (SEQ ID NO: 3) and 5′-TTGCCTTAAACAAGACCACGAAA (SEQ ID NO: 4)), Oct4 (5′-TCTTTCCACCAGG C CCCCGGCTC (SEQ ID NO: 5) and 5′-TGCGGGCGGACATGGGGAGATCC (SEQ ID NO: 6)), Rex1 (5′-ACGAGTGGCAGTTTCTTCTTGGGA (SEQ ID NO: 7) and 5′-TATGACTCACTTCCAGGGGGCACT (SEQ ID NO: 8)). The reverse primer (5′-TTTCTACAAGAAAGCTGGGT) (SEQ ID NO: 9) was used for all transgenes, plus the following forward primers: Nanog (5′-TTGGAATGCTGCTCCGCTCC) (SEQ ID NO: 10), Sox2 (5′-CTACAGCATGTCCTACTCGC) (SEQ ID NO: 11), Oct4 (5′-GCTATGGAAGCCCCCACTTC) (SEQ ID NO: 12), and Klf4 (5′-TGACTATGCAGGCTGTGGCA) (SEQ ID NO: 13). QPCR was performed using these primers and SYBR green (Bio-Rad). Additional primers for determining the level of expression are listed in Table 1.

TABLE 1

| Gene | Primer Seq | Use |
|---|---|---|
| AlphaMhc RT Forward | GAGATTTCTCCAACCCAG (SEQ ID NO: 14) | RT-PCR for |
| AlphaMhc RT Reverse | TCTGACTTTCGGAGGTACT (SEQ ID NO: 15) | A1phaMHC |
| Brachyury RT Forward | CTCCAACCTATGCGGACAAT (SEQ ID NO: 16) | RT-PCR for |
| Brachyury RT Reverse | CCCCTTCATACATCGGAGAA (SEQ ID NO: 17) | Brachyury |
| Cripto RT Forward | ATG GAC GCA ACT GTG AAC ATG ATG TTC GCA | RT-PCR for Cripto |
| | (SEQ ID NO: 18) | |
| Cripto RT Reverse | CTT TGA GGT CCT GGT CCA TCA CGT GAC CAT | |
| | (SEQ ID NO: 19) | |
| Flk1 RT Forward | GGCGGTGGTGACAGTATCTT (SEQ ID NO: 20) | RT-PCR for Flk1 |
| Flk1 RT Reverse | CTCGGTGATGTACACGATGC (SEQ ID NO: 21) | |
| Foxd3 RT Forward | ACCCTACTCTTACATCGCGCTCAT (SEQ ID NO: 22) | RT-PCR for Foxd3 |
| Foxd3 RT Reverse | AACGGTTGCTGATGAACTCGCAGA (SEQ ID NO: 23) | |
| Gata6 RT Forward | ACC TTA TGG CGT AGA AAT GCT GAG GGT G | RT-PCR for Gata6 |
| | (SEQ ID NO: 24) | |
| Gata6 RT Reverse | CTG AAT ACT TGA GGT CAC TGT TCT CGG G | |
| | (SEQ ID NO: 25) | |
| Gdf3 RT Forward | GTT CCA ACC TGT GCC TCG CGT CTT | RT-PCR for Gdf3 |
| | (SEQ ID NO: 26) | |
| Gdf3 RT Reverse | AGC GAG GCA TGG AGA GAG CGG AGC AG | |
| | (SEQ ID NO: 27) | |
| Gsc RT Forward | GACAGTCGATGCTACTTGCACACA (SEQ ID NO: 28) | RT-PCR for GSC |
| Gsc RT Reverse | AGCAGTCCTGGGCCTGTACATTAT (SEQ ID NO: 29) | |
| Hprt RT Forward | TACGAGGAGTCCTGTTGATGTTGC (SEQ ID NO: 30) | RT-PCR for Hprt |
| Hprt RT Reverse | GGGACGCAGCAACTGACATTTCTA (SEQ ID NO: 31) | |
| Islet1 RT Forward | ATGATGGTGGTTTACAGGCTAAC (SEQ ID NO: 32) | RT-PCR for Islet1 |
| Islet1 RT Reverse | TCGATGCACTTCACTGCCAG (SEQ ID NO: 33) | |
| Map2 RT Forward | CAT CGC CAG CCT CGG AAC AAA CAG | RT-PCR for Map2 |
| | (SEQ ID NO: 34) | |
| Map2 RT Reverse | TGC GCA AAT GGA ACT GGA GGC AAC | |
| | (SEQ ID NO: 35) | |
| Myf5 RT Forward | GAGCTGCTGAGGGAACAGGTGG (SEQ ID NO: 36) | RT-PCR for Myf5 |
| Myf5 RT Reverse | GTTCTTTCGGGACCAGACAGGG (SEQ ID NO: 37) | |
| MyoD RT Forward | AGGCTCTGCTGCGCGACCAG (SEQ ID NO: 38) | RT-PCR for MyoD |
| MyoD RT Reverse | TGCAGTCGATCTCTCAAAGC (SEQ ID NO: 39) | |
| MyoG RT Forward | TGAGGGAGAAGCGCAGGCTCAAG (SEQ ID NO: 40) | RT-PCR for MyoG |
| MyoG RT Reverse | ATGCTGTCCACGATGGACGTAAGG (SEQ ID NO: 41) | |
| Nkx2.5 RT Forward | CAAGTGCTCTCCTGCTTTCC (SEQ ID NO: 42) | RT-PCR for Nkx2.5 |
| Nkx2.5 RT Reverse | GGCTTTGTCCAGCTCCACT (SEQ ID NO: 43) | |
| Pax3 RT Forward | AACACTGGCCCTCAGTGAGTTCTAT (SEQ ID NO: 44) | RT-PCR for Pax3 |
| Pax3 RT Reverse | ACTCAGGATGCCATCGATGCTGTG (SEQ ID NO: 45) | |
| Pax6 RT Forward | TCACCATGGCAAACAACCTGCCTA (SEQ ID NO: 46) | RT-PCR for Pax6 |
| Pax6 RT Reverse | CATGGGCTGACTGTTCATGTGTGT (SEQ ID NO: 47) | |
| Pax7 RT Forward | CATCCAGTGCTGGTACCCCACAG (SEQ ID NO: 48) | RT-PCR for Pax7 |
| Pax7 RT Reverse | CTGTGGATGTCACCTGCTTGAA (SEQ ID NO: 49) | |
| Sox1 RT Forward | CCAAGATGCACAACTCGGAGATCA (SEQ ID NO: 50) | RT-PCR for Sox1 |
| Sox1 RT Reverse | TAATCCGGGTGTTCCTTCATGTGC (SEQ ID NO: 51) | |
| Nanog Forward | CAGGTGTTTGAGGGTAGCTC (SEQ ID NO: 52) | RT-PCR for Nanog |
| Nanog Reverse | CGGTTCATCATGGTACAGTC (SEQ ID NO: 53) | |
| Sox2 Forward | TAGAGCTAGACTCCG GGCGATGA (SEQ ID NO: 54) | RT-PCR for |
| Sox2 Reverse | TTGCCTTAAACAAGACCACGAAA (SEQ ID NO: 55) | endogenous Sox2 |
| Oct4 Forward | TCTTTCCACCAGGCCCCCGGCTC (SEQ ID NO: 56) | RT-PCR for |
| Oct4 Reverse | TGCGGGCGGACATGGGGAGATCC (SEQ ID NO: 57) | endogenous Oct4 |
| Oct4 (TG) Forward | GCTATGGAAGCCCCCACTTC (SEQ ID NO: 58) | RT-PCR for Oct4 |
| Oct4 (TG) Reverse | TTTGTACAAGAAAGCTGGGT (SEQ ID NO: 59) | Transgene |
| Klf4 (Tg) Forward | TGACTATGCAGGCTGTGGCA (SEQ ID NO: 60) | RT-PCR for Klf4 |
| Klf4 (Tg) Reverse | TTTGTACAAGAAAGCTGGGT (SEQ ID NO: 61) | Transgene |
| Sox2 (Tg) Forward | CTACAGCATGTCCTACTCGC (SEQ ID NO: 62) | RT-PCR for Sox2 |
| Sox2 (Tg) Reverse | TTTGTACAAGAAAGCTGGGT (SEQ ID NO: 63) | Transgene |
| cMyc (Tg) Forward | CAAGAGGCGGACACACAACG (SEQ ID NO: 64) | RT-PCR for cMyc |
| cMyc (Tg) Reverse | TTTGTACAAGAAAGCTGGGT (SEQ ID NO: 65) | Transgene |
| Nanog proximinal ChIP | GGAAGTGTCTTTAGATCAGAGG (SEQ ID NO: 66) | Nanog proximinal |
| Forward | | ChIP |
| Nanog proximinal ChIP | CCAAATCAGCCTATCTGAAGG (SEQ ID NO: 67) | |
| Reverse | | |
| Oct4 ChIP Forward | GTGAGGTGTCCGGTGACCCAAGGCAG (SEQ ID NO: 68) | Oct4 proximinal |
| Oct4 ChIP Reverse | CGGCTCACCTAGGGACGGTTTCACC (SEQ ID NO: 69) | ChIP |
| Oct4 (F1 Nested) | GTTGTTTTGTTTTGGTTTTGGATAT (SEQ ID NO: 71) | Methylation |
| Oct4 (F2 Nested) | ATGGGTTGAAATATTGGGTTTATTTA (SEQ ID NO: 72) | analysis of Oct4 |
| Oct4 R (Nested) | CCACCCTCTAACCTTAACCTCTAAC (SEQ ID NO: 73) | |
| Nanog F1 (Nested) | GAGGATGTTTTTTAAGTTTTTTTT (SEQ ID NO: 74) | Methylation |
| Nanog F2 (Nested) | AATGTTTATGGTGGATTTTGTAGGT (SEQ ID NO: 75) | analysis of Nanog |
| Nanog R (Nested) | CCCACACTCATATCAATATAATAAC (SEQ ID NO: 76) | |
| Sry Forward | TTGTCTAGAGAGCATGGAGGGCCATGTCAA (SEQ ID | Sry genotyping |
| | NO: 77) | |
| Sry Reverse | CCACTCCTCTGTGACACTTTAGCCCTCCGA (SEQ ID | |
| | NO: 78) | |

Reprogramming of Stable Intermediate Cell Lines by Viral Transduction

Oct4::GFP-negative cell lines were transduced using the same methodology and reagents as MEFs were in the original screen. Cells were infected with three rounds of viral supernatant diluted 1:8 in MEF media in a 48-hour period on gelatin. Two days after the last viral supernatant was added, the cells were trypsinized and replated onto feeders. The media was changed to mES media containing knockout serum replacement (KSR) instead of FBS on the following day.

Doubling Time Determination

piPSCs, iPSCs and mESC cell lines were seeded onto identical twelve well plates. To determine doubling time cells were grown in mESC culture medium supplemented with 15% fetal bovine serum. Approximately every twenty-four hours three well of the original plate were harvested and the number of cells present was determined using a hemacytometer. The counting procedure was repeated four times over 96-hours to generate growth curves. Doubling times for each cell line were then calculated using the growth curves.

Bisulfite Sequencing

piPS, iPS and MEF cell lines were grown to seventy percent confluency on gelatin coated 10 cm plates. Genomic DNA was harvested using Genomic DNA mini kit (Invitrogen) as per manufacturer's instructions. Bisulfite treatment of DNA was performed with the Epi Tect Bisulfite Kit (Qiagen) according to maufacturer's instructions. Primer sequences were as previously described for Oct4 (Blelloch et al., 2006) and Nanog (Takahashi and Yamanaka, 2006). Amplified products were purified by using gel filtration columns, cloned into pCR2.1-TOPO vector (Invitrogen) and sequenced with M13 forward and reverse primers.

In some instances, bisufite treatment of DNA was performed with EpiTect Bisulfite Kit (Qiagen) according to manufacturer's instructions. Primer sequences were as previously described for Oct4 (Blelloch et al., 2006) and Nanog (Takahashi and Yamanaka, 2006). Amplified products were purified using gel filtration columns, cloned into pCR2.1-TOPO vector (Invitrogen) and sequenced with M13 forward and reverse primers.

### Example 1

The majority of transduced cells fail to successfully navigate the sequential steps for attaining pluripotency and as a result become trapped in intermediate states (Mikkelson et al., 2008). The known partially reprogrammed states are characterized by unique epigenetic and molecular signatures such as DNA hypermethylation of critical pluriptency genes, low-level reactivation of endogenous stemness genes, and incomplete silencing of lineage-specifying transcription (Mikkelson et al., 2008; Sridharan et al., 2009). However, multiple yet to be described partially reprogrammed states may exist. Indeed, since the first description of partially reprogrammed cells that reprogrammed upon inhibition of DNA methylransferase with 5′-Aza cytodine (5′ AzaC), two additional populations that reprogram upon treatment with small molecule inhibitors of distinct signal transduction pathways, have been described (Ichida et al., 2009; Silva et al, 2008).

The first report on the derivation of murine iPSCs described a pluripotent cell capable of differentiating in vitro and in vivo to all three embryonic germ-layers. However, these first generations of iPSCs were unable to give rise to live chimeric offspring (Yamanaka and Takahashi et al., 2006). Improved reporters and selection criteria led to a second generation of iPSCs that give rise to live chimeric offspring with competent germ-line transmission (Maherali et al., 2007; Wernig et al., 2007; Okita et al., 2007). Subsequent work has generated iPSC lines with higher developmental potency that are capable of generating live offspring via tetraploid complementation (Zhao et al., 2009; Kang et al., 2009). A retrospective analysis of these third generation iPSCs has linked the inability to yield live offspring during tetraploid complementation to abherrent silencing of maternally imprinted genes (Stadtfeld et al., 2010). This indicated that there are multiple pluripotent states with varying degrees of potency that are molecularly and epigenetically unique.

A more detailed description of the epigenetic, molecular and pluripotent properties of partially reprogrammed cells that arise naturally during reprogramming would shed light on the molecular nature of reprogramming and differentiation.

Herein, the inventors have studied nine partially reprogrammed lines. The inventors have discovered that partially reprogrammed cells exhibit low level activation of endogenous pluripotency genes which corresponds to varying states of DNA methylation. In addition, partially reprogrammed cell lines exhibit differential responses to reprogramming small molecules. A number of the partially reprogrammed cells can be converted to a pluripotent state with small molecules that modulate distinct signal transduction events (Ichida et al., 2009; Silva et al., 2008). The ability to reprogram upon chemical induction also correlates with variable epigenetic modifications between cell lines. A number of partially reprogrammed lines are capable of robust directed differentiation to specific cell types. Interestingly, partially reprogrammed cells differ greatly in their ability and tendency to differentiate into specific germ lineages. Thus, the inventors have discovered a unique population of partially reprogrammed cells which are useful and provides novel insight into the stability, potential, and molecular basis of partially reprogrammed states.

### Example 2

Derivation of Partially Reprogrammed Cells Lines.

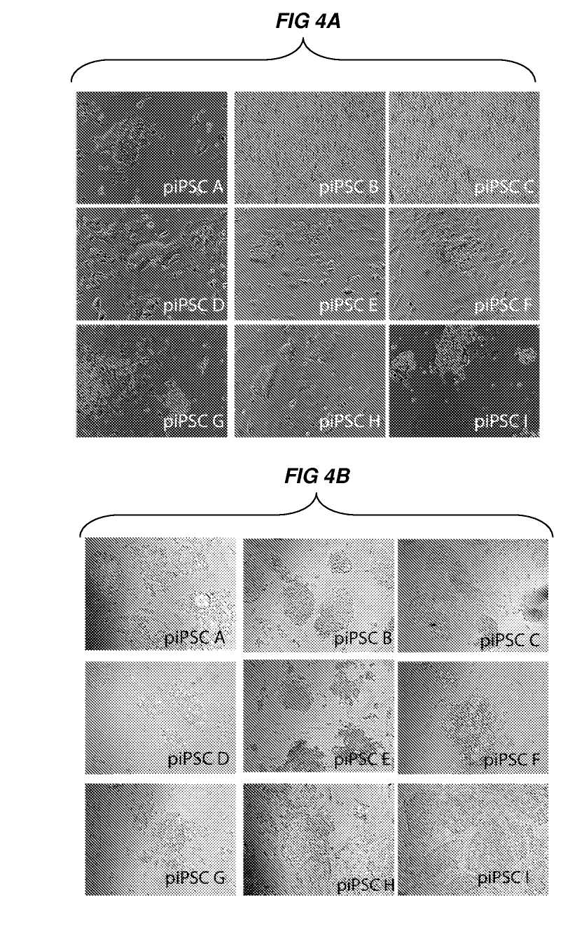

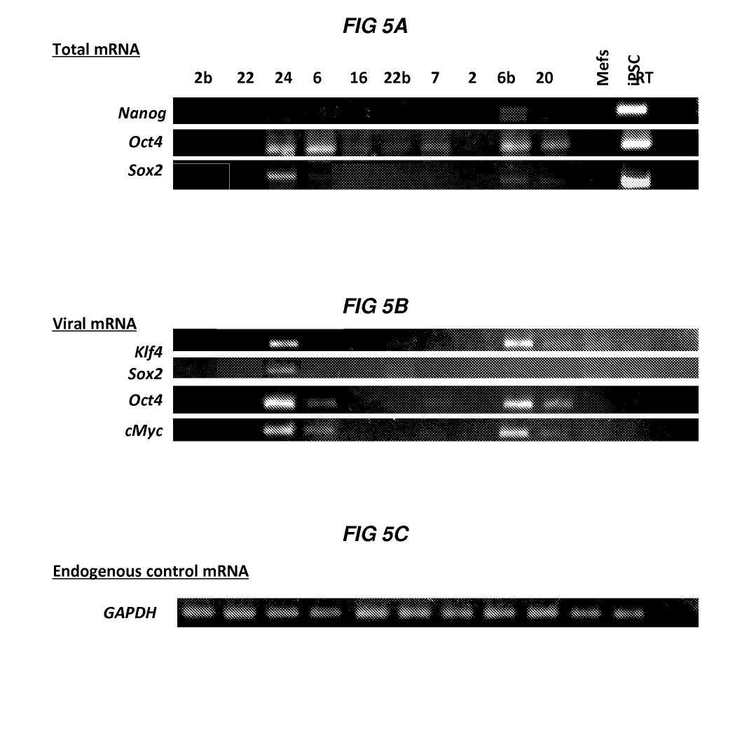

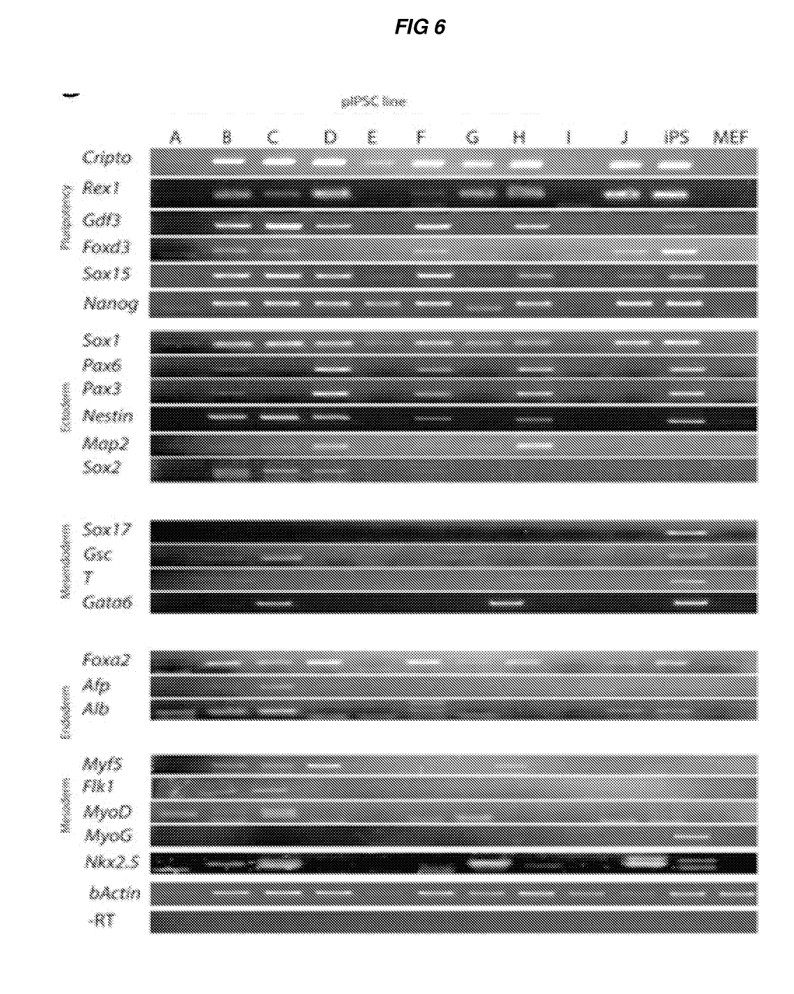

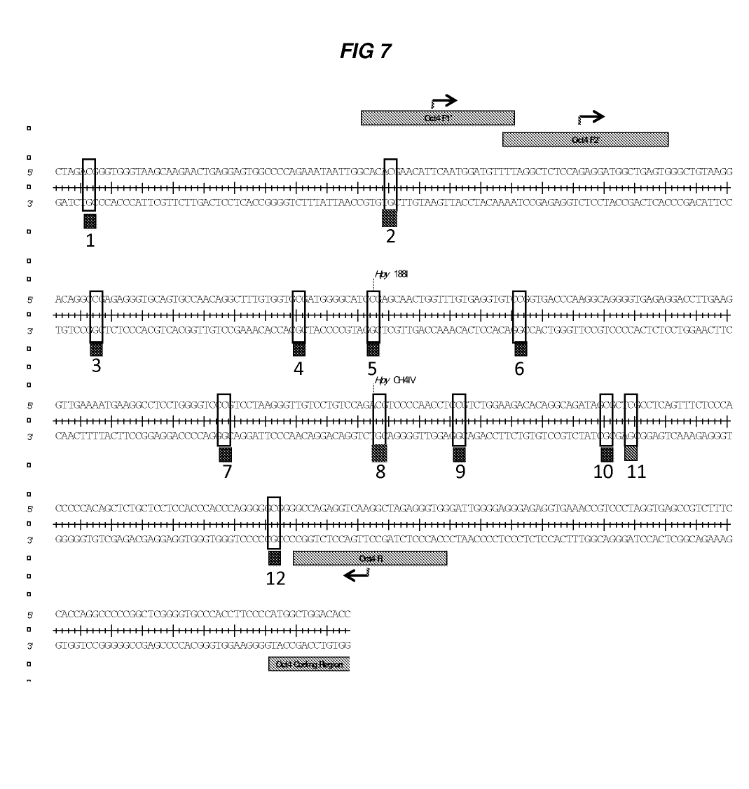

To generate partially reprogrammed cells for characterization, mouse embryonic fibroblasts (MEFs) harboring an Oct4::GFP transgene were virally transduced with Klf4, Sox2, Oct4, and cMyc. The inventors used normalized Gaussian distribution of reprogramming factors obtained via viral transduction would yield a more diverse set of intermediates than alternative methods where precise stoichiometery of individual reprogramming is controlled. Three weeks post infection, multiple partially (Oct4::GFP−) and fully (Oct4::GFP+) reprogrammed lines were clonally isolated and expanded into piPSC or iPSC cell lines respectively. Genotype analysis and RT-PCR confirmed each piPSC obtained from clonal selection contained copies that maintained persistent expression of each of the four reprogramming factors (FIG. 5B). Clonally expanded piPSC cell lines form colonies with a range of distinct morphologies (FIG. 23B-23C) that are all capable of self-renewing for greater than twenty passages without further reprogramming or undergoing senescence (FIGS. 4A and 4B)). This suggests the selected piPSC lines cultured under conventional mESC culture conditions are in a stable and conditionally immortalized state.

piPSCs and iPSCs were immunostained for transcription factors, Nanog, oct4, Sox2 (data not shown). piPS cells were cultured on irradiated MEF feeders in chamber slides, fixed with 4% PFA and stained with primary antibodies against mSox2 (Santa Cruz, sc-17320), mNanog (CosmoBio, REC-RCAB0002 PF), followed by staining with the appropriate secondary antibodies conjugated to Alexa Fluor 546 (Invitrogen). Nuclei were counterstained with Hoechst33342 (Sigma). iPS cells were assayed for alkaline phosphatase activity using the Vector Red alkaline phosphatase assay kit from Vector Laboratories. Only iPSCs were positively immunostained for Nanog, whereas while strong immunostaining was detected for Oct4 and Sox2 in iPSCs, low level of expression for Oct4 and Sox2 was detected in the piPSC lines (data not shown).

### Example 3

piPSC Lines Exhibit Heterogeneous Expression of Early Reprogramming Markers and Distinct Proliferative Rates.

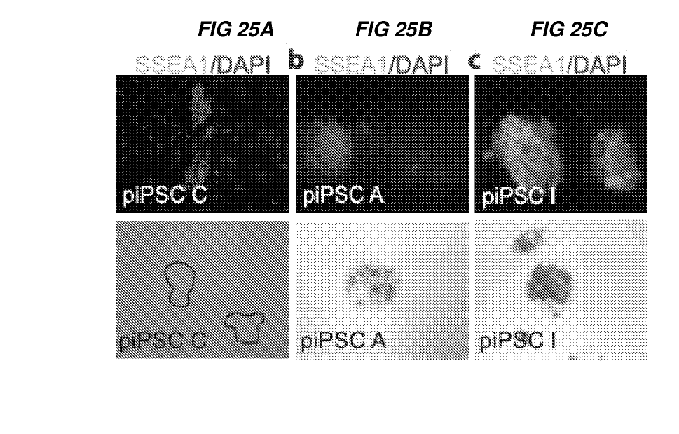

To characterize differences between piPSC and iPSC we first immunostained for the early reprogramming markers alkaline phosphatase (AP) and SSEA1. Using these markers, piPSC lines were categorized into three expression and morphological profiles of the early reprogramming markers SSEA1 and AP, as defined by Table 2 and shown in FIG. 25A-25B.

TABLE 2
Three types of piPS cell lines based on expression
and morphological characterization profiles

| piPS cell | Alkaline phosptatase | | Morphological |
|---|---|---|---|
| type | (AP) expression | SSEA expression | characteristic |
| Type (i) | Negative AP expression | Negative SSEA | Small granular |
| | | expression | |
| Type (ii) | Positive AP expression | Negative SSEA | Compact |
| | (diffuse expression) | expression | colonies |
| Type (iii) | Strong Positive AP | Strong postive | Compact |
| | expression | SSEA expression | colonies |

### Example 2

Stable Reprogrammed Intermediate (piPS) Cell Lines Exhibit Distinct Molecular Signatures from iPSCs and MEFs.

Previous microarray-based expression profiling allegedly reports presence of possible intermediate reprogrammed lines (Mikkelsen et al, 2008, Sridharan et al, 2009). However, Mikkelsen et al., and Sridharan et al., were unable to specifically identify or isolate the intermediate reprogrammed cells, or detect activation of endogenous pluripotency programs. Since subtle distinctions in gene expression may delineate the differences between the stable intermediate piPSCs as disclosed herein and MEFs and iPSCs, the inventors employed real-time TaqMan RT-PCR to demonstrate the molecular profile to distinguish the stable intermediate piPSCs as disclosed herein from MEFs and iPSCs and to quantify the gene expression of a subset of ninety genes associated with pluripotency, stemness, and lineage specification in the stable intermediate piPSCs.

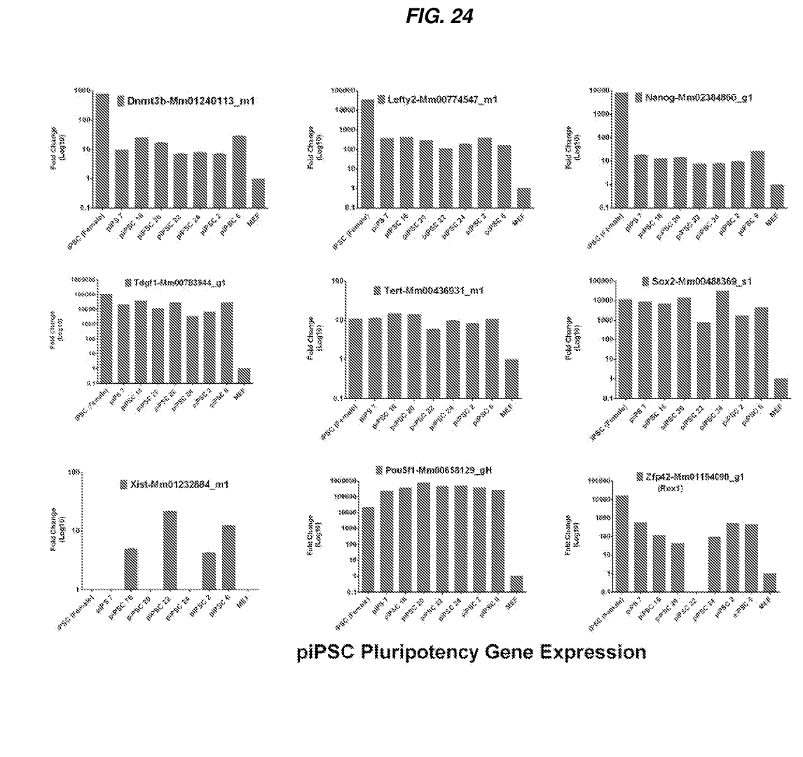

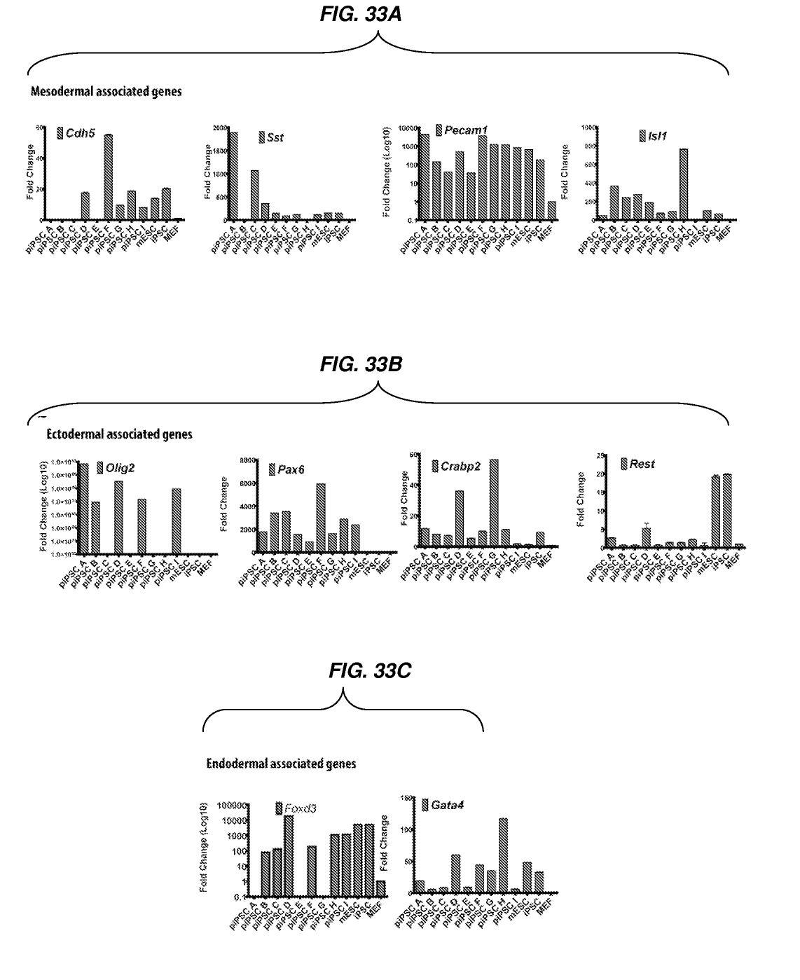

As shown in FIGS. 24, 26A-26J, and 33A-33B, PiPS cells can be characterized on the expression profile as compare to iPS cells or mES cells or MEFs.

Stable Reprogrammed Intermediate (piPS) Cells are Epigenetically Distinct from iPS Cells and MEFs.

To Examine the epigenetic status of multiple piPSC lines, the inventors performed bisulfide sequencing on the DMR of the pluripotency genes Oct4 and Nanog. As shown in FIG. 27A, all piPSC lines examined express significantly lower levels of the de novo methyl-transferase Dnmt3b than iPSC, but significantly greater than MEFs (p<0001). Similarly, piPSC exhibit intermediate levels of DNA methylation on the differentially methlyated region of Oct4 and Nanog (FIGS. 27B and 27C)

### Example 3

Stable Reprogrammed Intermediate (piPS) Cells Respond Differently to Reprogramming Chemicals.

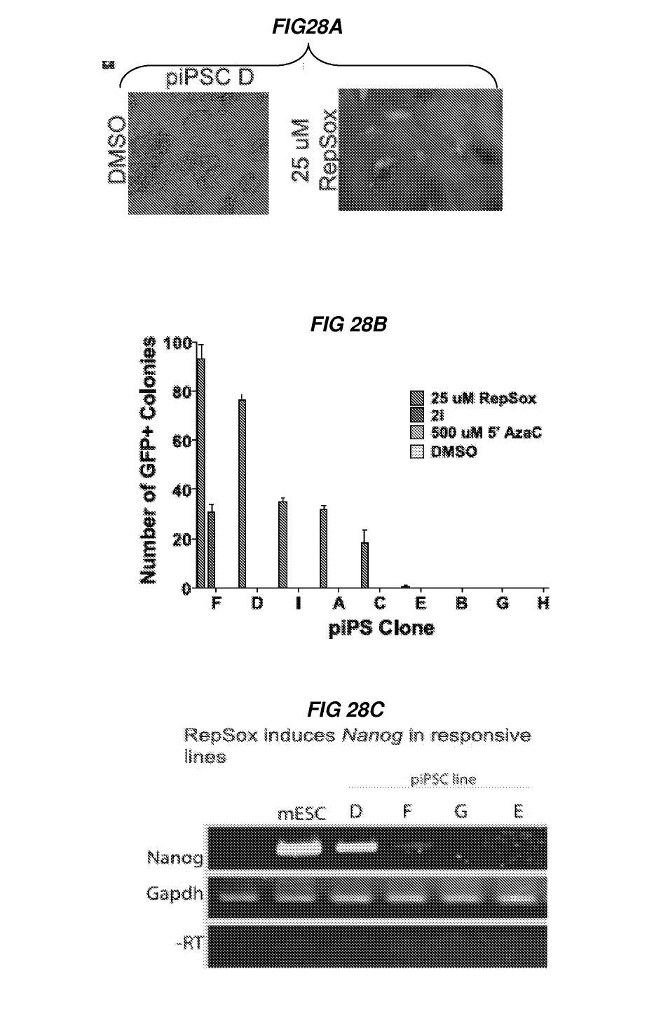

To examine whether multiple piPSC lines could be further reprogrammed to a fully pluripotent state, piPSC were cultured in the presence of RepSox, a known inhibitor of Tgf-β signalling previously described to reprogram intermediate cells (Ichida et al., 2009), 2i-treatment, combined chemical inhibition of Mek/Erk and GSK3 signaling (Silva et at, 2008), or the DNA methyl-transferase inhibitor 5′AzaC. As shown in FIG. 28B, numerous stable intermediate cell lines (piPSC lines D, F, G, and E) underwent further reprogramming to fully reprogrammed iPS cells with treatment of 25 μM Repsox for 48 hours as determined by the expression of Nanog (FIGS. 28A and 28C).

Chemically Reprogrammable Stable Reprogrammed Intermediate (Pips) Cell Lines have Higher Levels of Activating Chromatin Associated with Nanog.

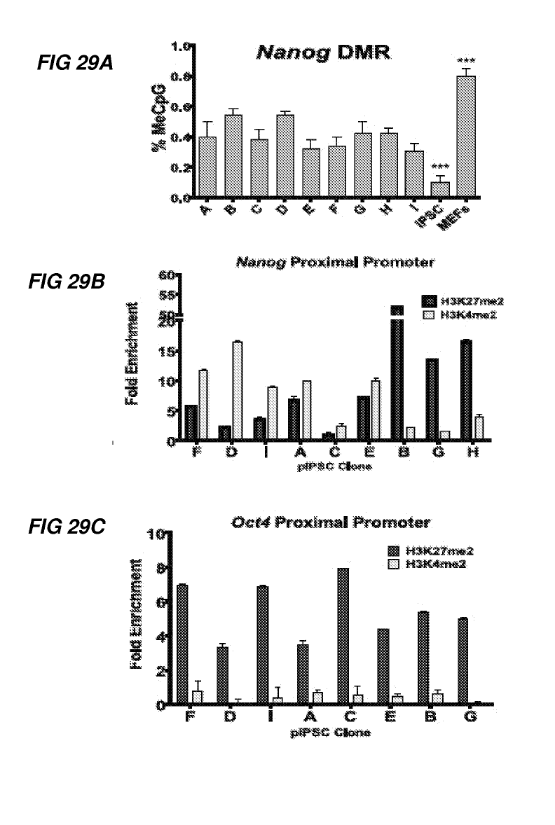

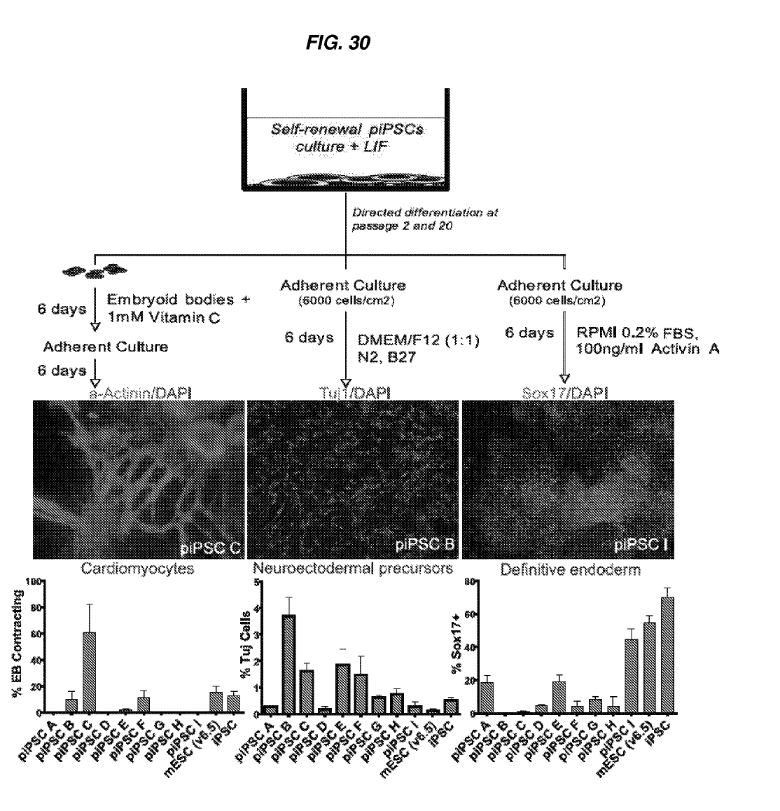

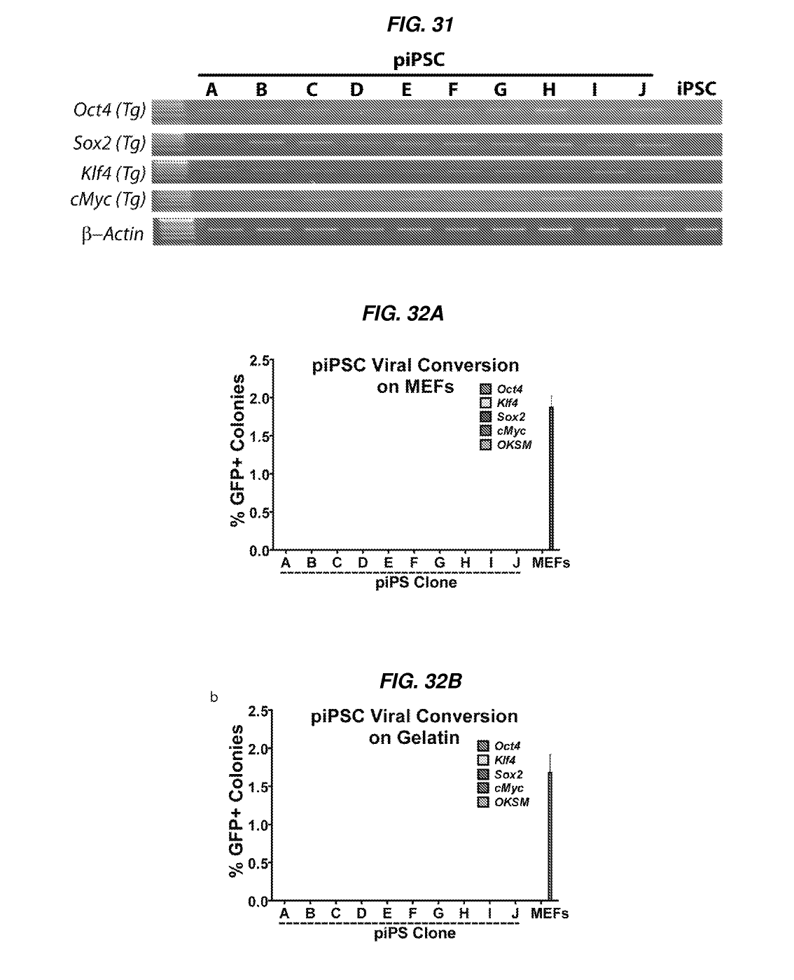

Since piPSC lines exhibit comparable pluripotency programs by RT-PCR, the inventors assessed the ability to reprogram upon chemical treatment was regulated by epigenetic modifications encoded on the Nanog promoter region through either DNA methylation or histone modifications. FIG. 29A-29C demonstrates that the reprogramming response of stable intermediate cell lines is determined by the level of histone modification associated with the Nanog promoter. While the DNA methylation of Nanog does not differ significantly (p<0.0001) between responsive and non-responsive stable intermediate reprogrammed (piPS) cell lines (FIG. 29A), the chromatin state of Nanog proximal promoter as determined by anti-H3K4me2 or anti-H3K27me2 antibodies indicates that specific stable intermediate reprogrammed (piPS) cell lines which can be further reprogrammed to fully reprogrammed cells have a high level of H3K4me2 antibody staining and a low level of anti-H3K4me2 staining (FIG. 29B). The inventors demonstrated that while the Oct4 promoter exhibits higher levels of H3K27 dimethylation as compared to H3K4 dimethylation, it was relatively the same levels in all stable intermediate reprogrammed (piPS) cell lines examined (FIG. 29C).

All piPSC Lines can Spontaneously Differentiate In Vitro and In Vivo.

Since piPSC lines express a semi-pluripotent transcriptional program, the inventors assessed if the stable intermediate reprogrammed (piPS) cell lines are be capable of multilineage differentiation. To characterize the ability of piPSC lines to give rise to more differentiated tissue, the inventors first examined the ability to spontaneously differentiate in vitro and in vivo. As shown in FIG. 11, piPSCs can differentiate in vivo and in vitro and readily form embryoid bodies. The stable intermediate reprogrammed (piPS) cell lines also undergo spontaneous differentiation to give rise to tissues that stains positive for Sox17, skeletal muscle actin, and neuronal tubulin I11 (Tuj 1), markers of the endodermal, mesodermal and ectodermal lineages, respectively (data not shown). Immunostaining of Sox17, MF20 (smooth muscle actin marker), and Tuji (neuronal marker) was detected (data not shown). iPS cells were assayed for alkaline phosphatase activity using the Vector Red alkaline phosphatase assay kit from Vector Laboratories (data not shown).

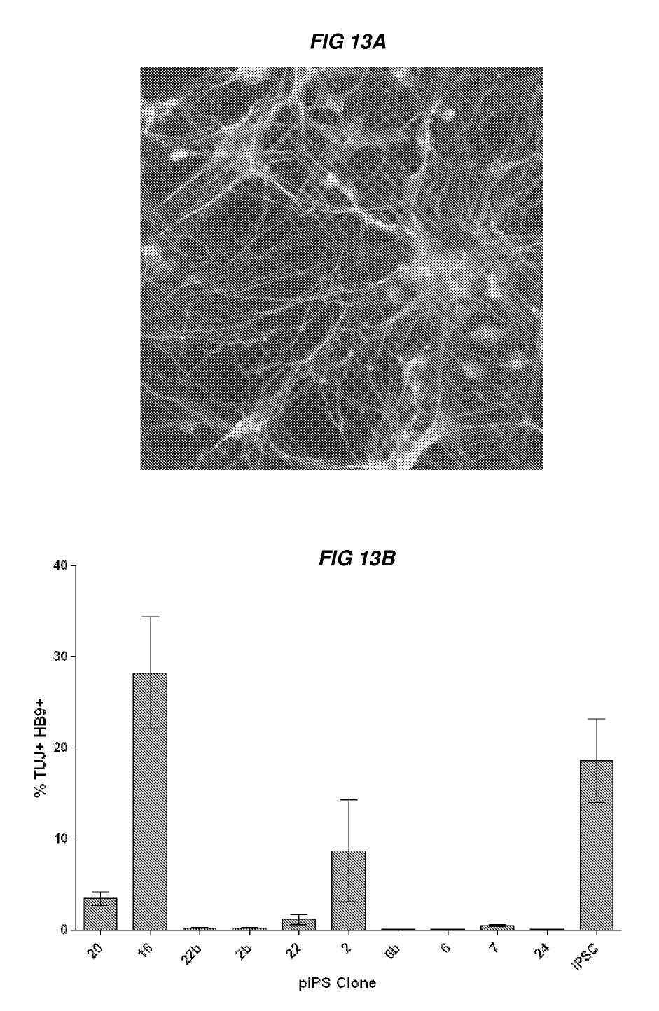

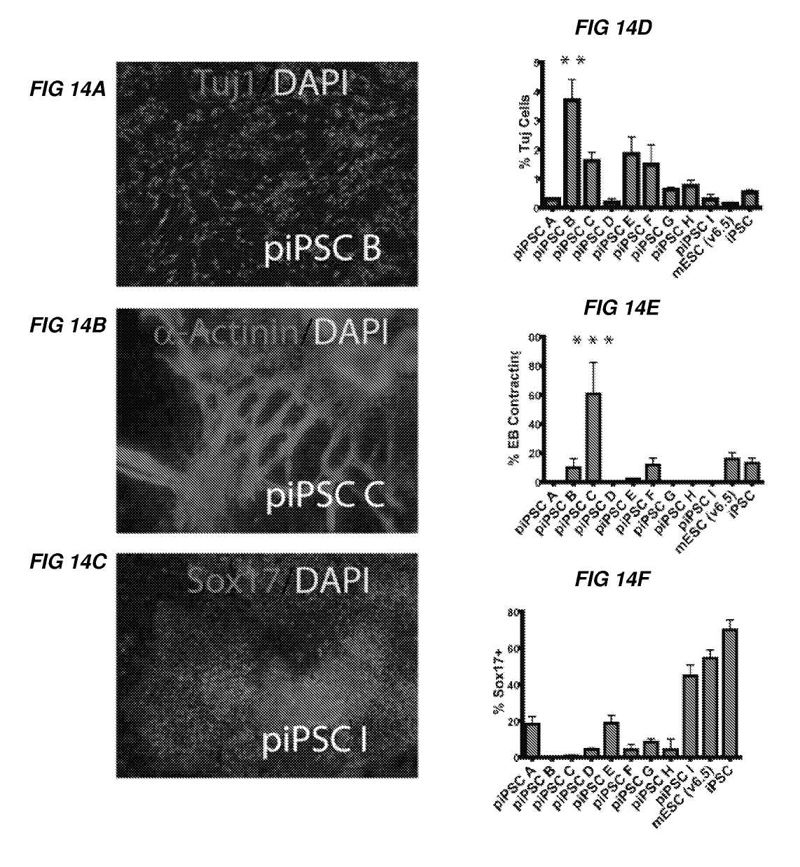

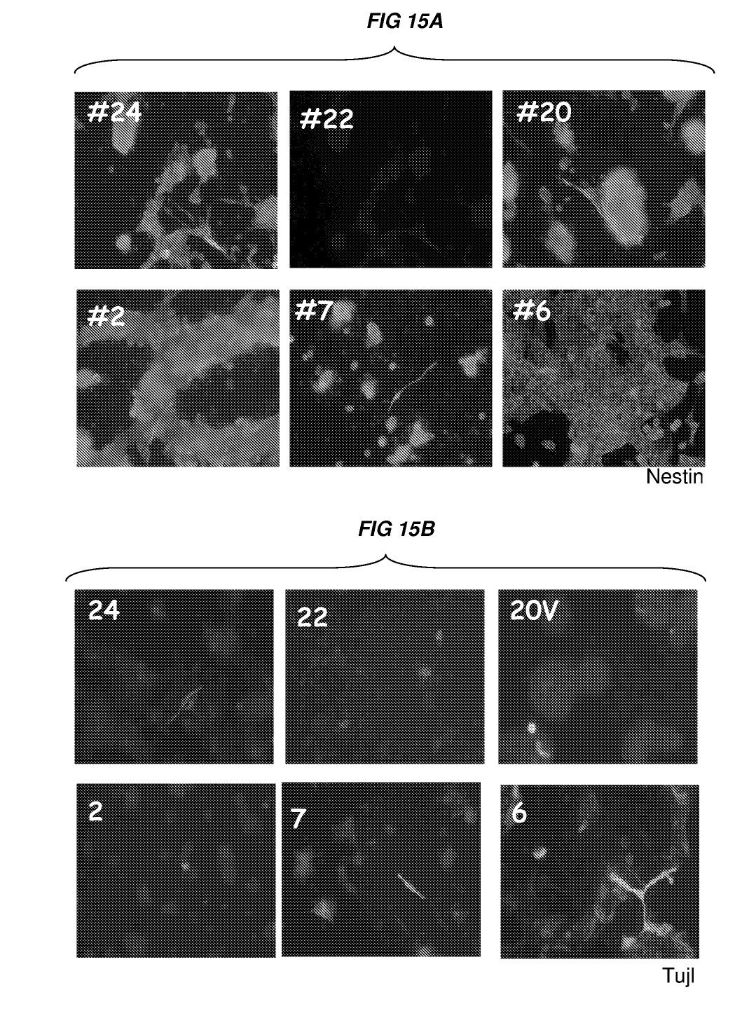

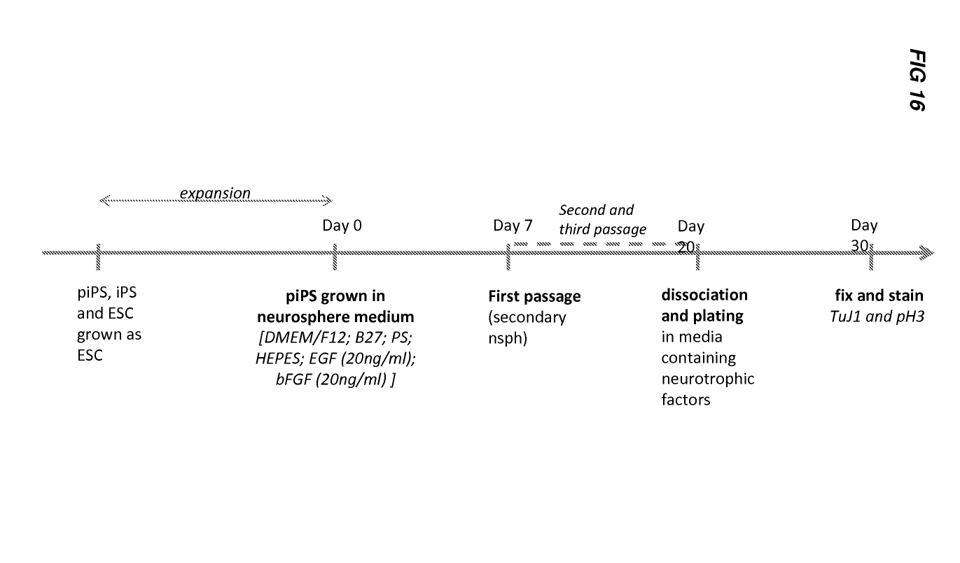

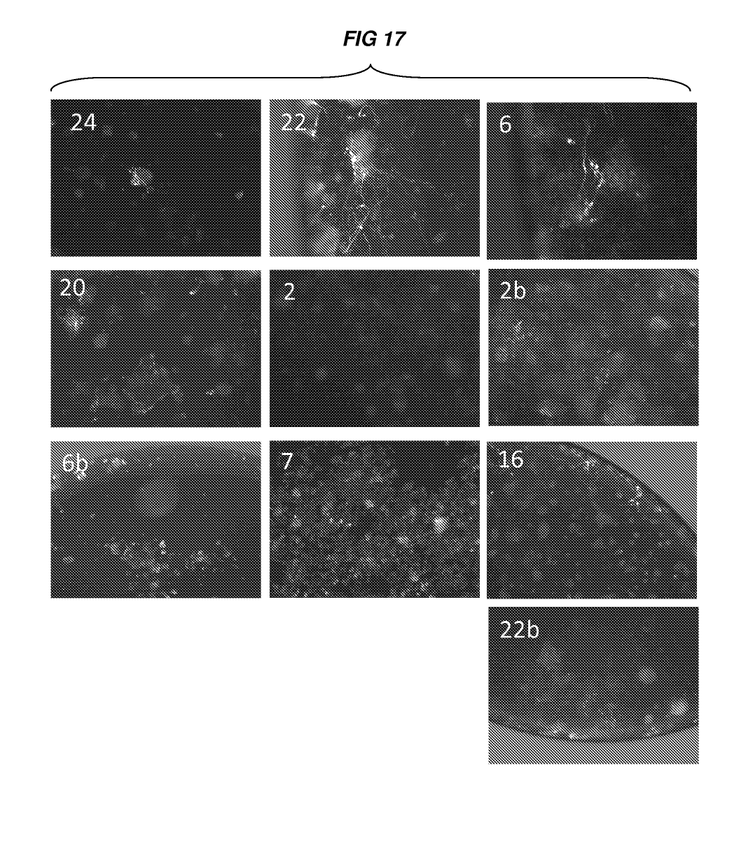

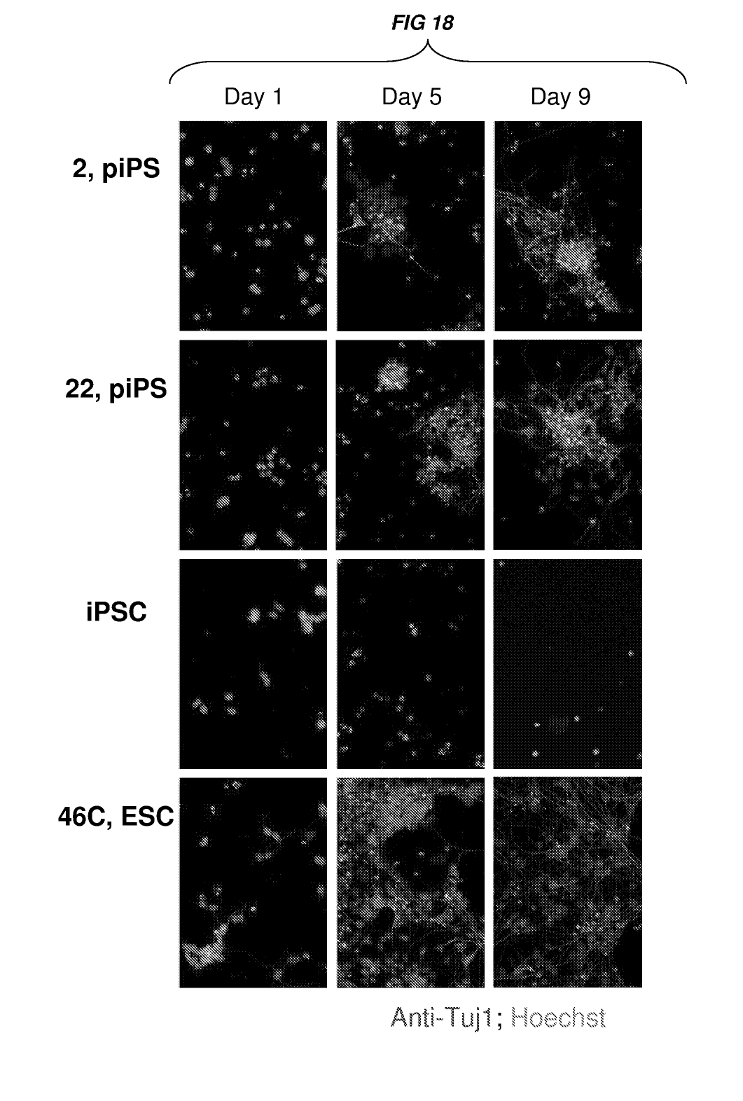

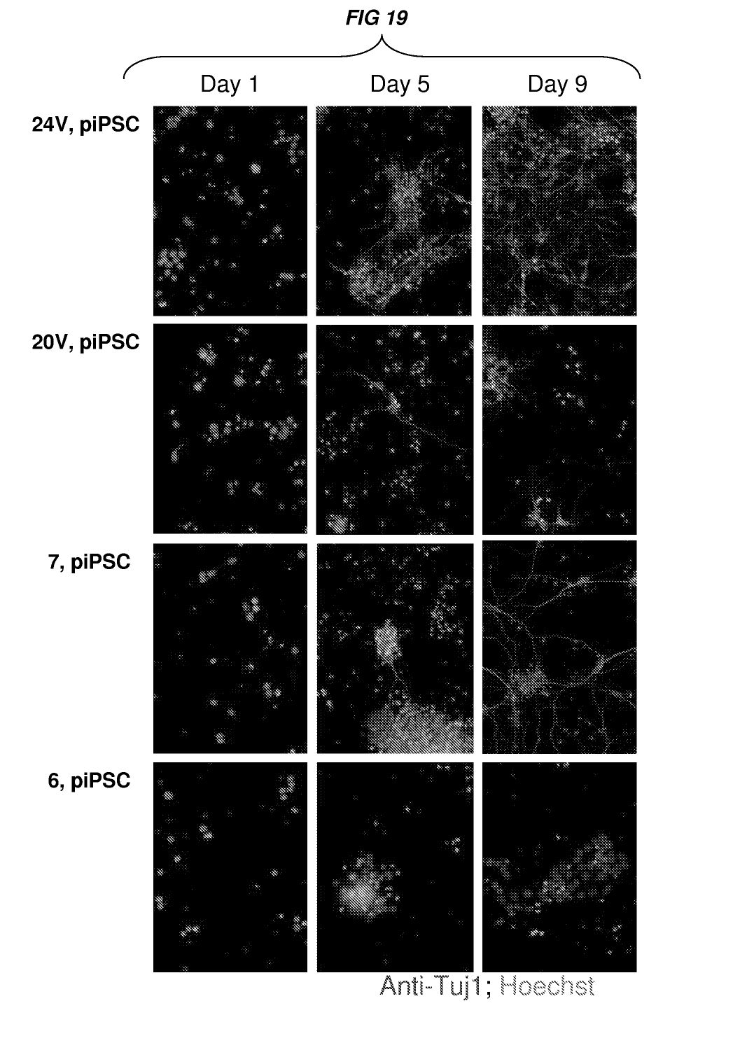

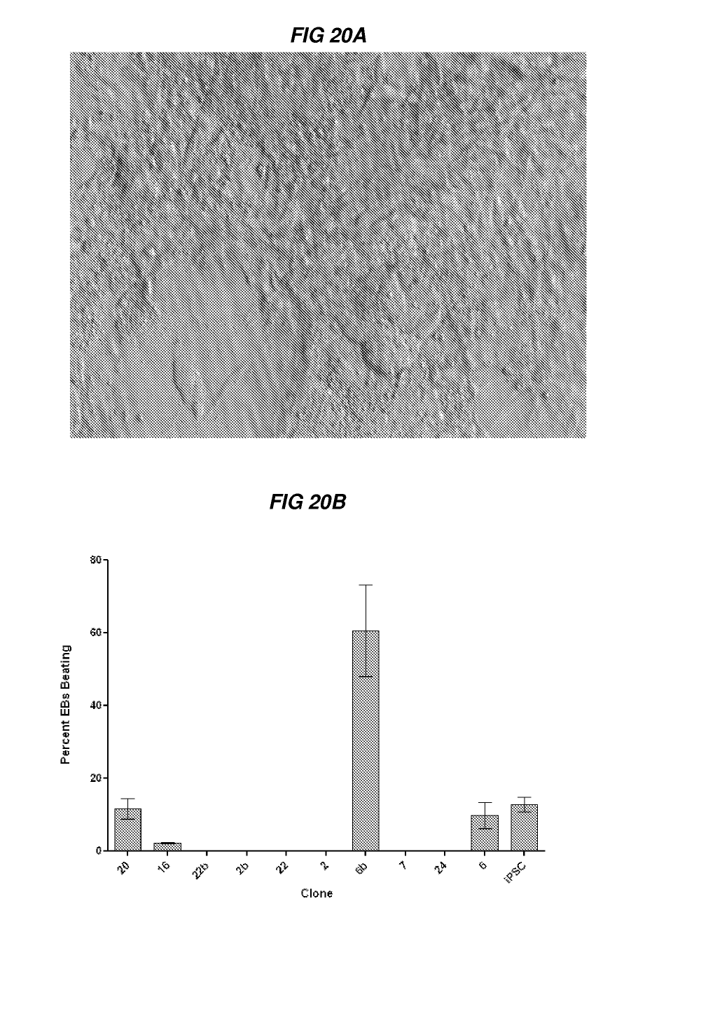

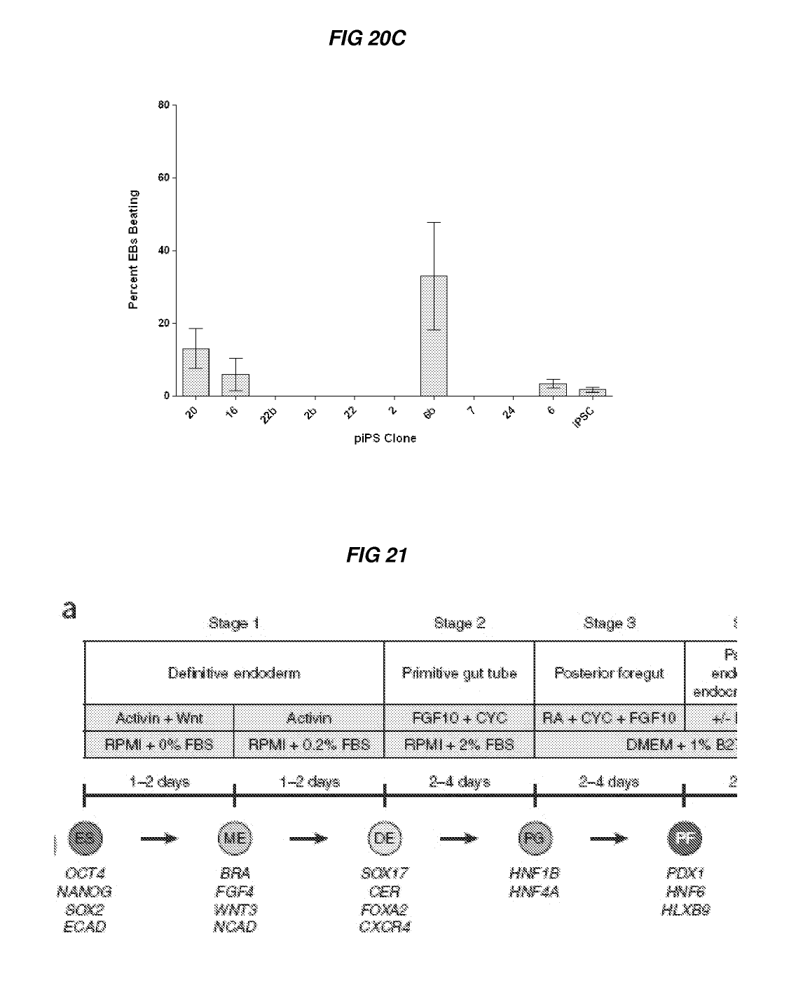

Furthermore, the inventors demonstrate that stable intermediate reprogrammed (piPS) cell lines can be directed to differentiate into post-mitotic motor neurons (FIG. 13A). The inventors demonstrate that the stable intermediate reprogrammed (piPS) cell lines are pluripotent and can form teratomas containing tissue from the three embryonic germ layers upon injection into nude mice. piPSCs form encapsulated teratomas that are immature and of a lower grade as compared to teratomas formed by iPSC or mESC.

### Example 4

piPSC Lines Exhibit a Differential Capacity for Directed Differentiation to Specific Cell Types.

To assess piPSCs response to standard in vitro inductive cues commonly employed for directed differentiation, piPSCs were simultaneously divided and directed to differentiate into cell types from each lineage, specifically contractile cardiomyocytes (mesoderm), neuroectodermal precursors (ectoderm), and definitive endoderm.

Effectively, the inventors herein have discovered a stable intermediate reprogrammed (piPS) cell which can be further reprogrammed to a fully and terminally reprogrammed cell (where a fully reprogrammed cell cannot be further reprogrammed). The stable intermediate reprogrammed (piPS) cell lines are in a stable and conditionally immortalized state and have an intermediate molecular and epigenetic states which are significantly distinct from both parental fibroblasts from which they are derived and fully reprogrammed iPS cells or ES cells. The inventors also demonstrate that different stable intermediate reprogrammed (piPS) cell lines respond differentially to reprogramming chemicals (RepSox, 2i, and 5′ Aza C), and that the stable intermediate reprogrammed (piPS) cell lines which are chemically reprogrammable have a significantly higher level of activating chromatin associated with Nanog but not Oct4. Additionally, the inventors demonstrate that stable intermediate reprogrammed (piPS) cell line can spontaneously differentiate across multiple lineages, both in vitro and in vivo, and can be directed to differentiate into all three lineages (endodermal, mesodermal and ectodermal lineages) by direct differentiation. In some instances, some stable intermediate reprogrammed (piPS) cell lines exhibit a preferential bias towards selected lineages upon directed differentiation (see FIG. 26A-26B).

The inventors discovery of stable intermediate reprogrammed (piPS) cell lines are distinct, both morphologically, epigenetically and molecularly from fully reprogrammed iPS cells (which cannot be reprogrammed further), and are useful in assays in studying the fundamental molecular mechanisms by which cells are reprogrammed and differentiate, as well as useful in assays for disease modeling to provide insights diseases and therapeutic applications. As stable intermediate reprogrammed (piPS) cell lines appear to preserve epigenetic modification specific to disease states, use of the stable intermediate reprogrammed (piPS) cell lines are advantageous for certain application in disease modeling, where epigenetic changes of specific genes contributes to the disease or disorder, as well as directing the differentiation of specific stable intermediate reprogrammed (piPS) cell lines along specific target cell types.

The inventors herein have demonstrated and isolated a population of partially reprogrammed intermediate cells that appeared 1-2 weeks after virus addition. Accordingly, herein the inventors have demonstrated that reprogramming can be achieved in several or numerous ways, with cells traversing different paths of dedifferentiation via many transient states of partial dedifferentiation. These reprogramming intermediates, referred to herein as stable partially programmed cells (or piPSCs) serve as artificial cells, e.g., being created as a result of an artificial process.

As disclosed herein, the inventors have demonstrated that fibroblasts need not be dedifferentiated completely to become other cell types. The potential advantage of this more direct approach, at least from a drug discovery perspective, is that more of the epigenetic modifications of patient-derived cells are preserved by bypassing complete reprogramming. Also, as disclosed herein, it is possible that piPSCs can be used in methods to readily produce more mature cells than one obtained when cells are reprogrammed completely and complete reversion of cells to a more embryonic cell-like state (e.g., iPSCs). Thus, piPSC are advantageous over using iPSCs, as piPSCs can be used to producing cell that more accurately model components of different diseases, and for therapeutic uses.

### REFERENCES

The references cited throughout the specification and Examples are incorporated herein in their entirety by reference.
Bernstein, B. E. et al. (2006). A bivalent chromatin structure marks key developmental genes in embryonic stem cells. Cell 125, 315-326.
Brambrink, T. et al (2008). Sequential expression of pluripotency markers during direct reprogramming of mouse somatic cells. Cell Stem Cell 2, 151-159.
Chan, E M., et al (2009). Live cell imaging distinguishes bona fide human iPS cells from partially reprogrammed cells. Nat Biotechnol 27, 1033-1037.
Hanna, J., et al. (2009). Direct cell reprogramming is a stochastic process amenable to acceleration. Nature 462, 595-601.
Ichida, J. K., et al (2009). A small-molecule inhibitor of tgf-Beta signaling replaces sox2 in reprogramming by inducing nanog. Cell Stem Cell 5, 491-503.
Kang, L., et al (2009). iPS cells can support full-term development of tetraploid blastocyst-complemented embryos. Cell Stem Cell 5, 135-138.
Maherali, N., et al (2007). Directly reprogrammed fibroblasts show global epigenetic remodeling and widespread tissue contribution. Cell Stem Cell 1, 55-70.
Mikkelsen, T. S., et al. (2008). Dissecting direct reprogramming through integrative genomic analysis. Nature 454, 49-55.
Okita, K., et al (2007). Generation of germline-competent induced pluripotent stem cells. Nature 448, 313-317.
Silva, J., et al (2008). Promotion of reprogramming to ground state pluripotency by signal inhibition. PLoS Biol 6, e253.
Silva, J., et al (2006). Nanog promotes transfer of pluripotency after cell fusion. Nature 441, 997-1001.
Silva, J., et al. (2009). Nanog is the gateway to the pluripotent ground state. Cell 138, 722-737.
Smith, Z. D., Nachman, I., Regev, A., and Meissner, A. Dynamic single-cell imaging of direct reprogramming reveals an early specifying event. Nat Biotechnol 28, 521-526.
Sridharan, R., et al (2009). Role of the murine reprogramming factors in the induction of pluripotency. Cell 136, 364-377.
Stadtfeld, M., Maherali, N., Breault, D. T., and Hochedlinger, K. (2008). Defining molecular cornerstones during fibroblast to iPS cell reprogramming in mouse. Cell Stem Cell 2, 230-240.
Takagi, N., et al. (1983). Reversal of X-inactivation in female mouse somatic cells hybridized with murine teratocarcinoma stem cells in vitro. Cell 34, 1053-1062.
Takahashi, K., and Yamanaka, S. (2006). Induction of pluripotent stem cells from mouse embryonic and adult fibroblast cultures by defined factors. Cell 126, 663-676.
Wernig, M., et al. (2007). In vitro reprogramming of fibroblasts into a pluripotent ES-cell-like state. Nature 448, 318-324.
Ying, Q L., et al, (2003). Conversion of embryonic stem cells into neuroectodermal precursors in adherent monoculture. Nature Biotech. 21, 183-186.
Zhao, X. Y., et al (2009). iPS cells produce viable mice through tetraploid complementation. Nature 461, 86-90.

1. An isolated reprogrammed cell that is capable of self renewing for at least twenty passages before senescence, wherein the isolated reprogrammed cell is a differentiated cell that has been reprogrammed to a less differentiated state and wherein the isolated cell is capable of being further reprogrammed to a less differentiated state.

2. The reprogrammed cell of claim 1, wherein the cell can differentiate into all three primary germ layer lineages selected from; endoderm lineage, mesoderm lineage and ectoderm lineage.

3.-8. (canceled)

9. The reprogrammed cell of claim 1, wherein the reprogrammed cell has at least a 100-fold lower expression of Dnm3b as compared to the level of expression of an induced pluripotent stem (iPS) cell.

10.-11. (canceled)

12. The reprogrammed cell of claim 1, wherein the reprogrammed cell has a lower expression of at least 2 of the following genes endogenous Oct4, endogenous Nanog, endogenous Rex1, endogenous Tdgf1 by a statistically significant amount relative to the level of expression of an induced pluripotent stem (iPS) cell.

13.-16. (canceled)

17. The reprogrammed cell of claim 1, wherein the reprogrammed cell has a lower expression of endogenous Lefty 2 by a statistically significant amount relative to the level of expression of an induced pluripotent stem (iPS) cell.

18.-21. (canceled)

22. The reprogrammed cell of claim 1, wherein the reprogrammed cell has a higher level of expression of any one of endogenous TGDF1, endogenous Tert or endogenous Sox2 by a statistically significant amount relative to the level of expression of a somatic cell.

23.-34. (canceled)

35. The reprogrammed cell of claim 1, wherein the reprogrammed cell has an increased CpG methylation of Oct4 or Nanog or Oct4 and Nanog a statistically significant amount relative to the level of CpG methylation of Oct4 or Nanog or Oct4 and Nanog an induced pluripotent stem (iPS), and/or a decreased CpG methylation of Oct4 or Nanog or Oct4 and Nanog a statistically significant amount relative to the level of CpG methylation of Oct4 or Nanog or Oct4 and Nanog and the isogenic cell from which the reprogrammed cell was derived.

36. The reprogrammed cell of claim 1, wherein the reprogrammed cell has an increased rate of proliferation by a statistically significant amount relative to the level of expression of an induced pluripotent stem (iPS) cell.

37.-47. (canceled)

48. The reprogrammed cell of claim 1, wherein the reprogrammed cell can be further reprogrammed into an induced pluripotent stem (iPS) cell.

49.-63. (canceled)

64. An isolated reprogrammed cell of claim 1, wherein the cell is isolated by selection based on at least one or any combination of the of following characteristics:
i. the reprogrammed cell can differentiate into all three primary germ layer lineages selected from; endoderm lineage, mesoderm lineage and ectoderm lineage;
ii. the reprogrammed cell has a doubling time of less than 15 hours;
iii. the reprogrammed cell has at least a 100-fold lower expression of Dnm3b as compared to the level of expression of an induced pluripotent stem (iPS) cell;
iv. the reprogrammed cell has at least a 100-fold lower expression of endogenous Nanog as compared to the level of expression of an induced pluripotent stem (iPS) cell;
v. the reprogrammed cell has at least a 100-fold lower expression of endogenous Lefty 2 as compared to the level of expression of an induced pluripotent stem (iPS) cell;
vi. the reprogrammed cell has at least a 1,000-fold higher expression of endogenous Tgdf1 as compared to the level of expression of endogenous Tgdf1 in a somatic cell;
vii. the reprogrammed cell has at least a 5-fold higher expression of endogenous Tert as compared to the level of expression of endogenous Tert in a somatic cell.
viii. the reprogrammed cell has at least a 100-fold higher expression of endogenous Sox2 as compared to the level of expression of endogenous Sox2 in a somatic cell;
ix. the reprogrammed cell has a lower expression of Krt10 or Thy1 or Krt10 and Thy1 by a statistically significant amount relative to the level of expression of the isogenic cell from which the reprogrammed cell was derived;
x. the reprogrammed cell has an increased CpG methylation of Oct4 or Nanog or Oct4 and Nanog by a statistically significant amount relative to the level of CpG methylation of Oct4 or Nanog or Oct4 an Nanog an induced pluripotent stem (iPS);
xi. the reprogrammed cell has an decreased CpG methylation of Oct4 or Nanog or Oct4 and Nanog a statistically significant amount relative to the level of CpG methylation of Oct4 or Nanog or Oct4 and Nanog and the isogenic cell from which the reprogrammed cell was derived; and
xii. the reprogrammed cell has at least a 5-fold higher expression of Xist in female lines as compared to the level of expression of endogenous Xist in a female induced pluripotent stem (iPS) cell.

65.-70. (canceled)

71. An isolated heterogeneous population of reprogrammed cells comprising at least two different reprogrammed cells of claim 1.

72. The isolated heterogeneous population of claim 71, comprising at least three different reprogrammed cells of claim 1.

73. The isolated heterogeneous population of claim 71, wherein the different reprogrammed cell populations are selected from the groups consisting of: (i) alkaline phosphatase negative, SSEA1 negative, small granular colonies, (ii) alkaline phosphatase positive, SSEA1 negative, compact colonies, and (iii) alkaline phosphatase positive, SSEA1 positive, compact colonies.

74. The isolated heterogeneous population of claim 71, wherein the population comprises less than 10% of iPS cells.

75.-83. (canceled)

84. A differentiated cell derived from inducing the differentiation of a reprogrammed cell of claim 1.

85. (canceled)

86. A method for stem cell therapy comprising;
i. isolating and collecting a somatic cell from a subject;
ii. reprogramming the somatic cell to a reprogrammed cell of claim 1;
iii. inducing differentiation of the reprogrammed cell of step (ii); and
iv. transplanting the differentiated cell from step (iii) into the subject.

87. A method to isolate a reprogrammed cell of claim 1 from a population of cells comprising induced pluripotent stem (iPS) cells and somatic cells, the method comprising:
i. positively selecting for cells with a statistically significant high level of expression at least one of Tdgf1, Tert, Sox2, Pou5f1;
ii. selecting the cells obtained in step (i) for cells with a significantly low level of expression of at least one of Dnmt3b, Dnmt3a, Rex1, Left2, or Nanog
wherein the selected cells in step (ii) are an isolated reprogrammed cell.

88. A method to isolate a reprogrammed cell of claim 1, from a population of cells comprising induced pluripotent stem cells (iPSCs), the method comprising positively selecting for cells with a significantly low level of expression of at least one of Dnmt3b, Dnmt3a, Rex1, Left2, or Nanog.

89. A method to isolate a reprogrammed cell of claim 1, from a population of cells comprising induced pluripotent stem cells (iPSCs), the method comprising negatively selecting for cells with a significantly high level of expression of at least one of Dnmt3b, Dnmt3a, Rex1, Left2, or Nanog, wherein the cells with a significantly high level of expression of at least one of Dnmt3b, Dnmt3a, Rex1, Left2, or Nanog are discarded.

90. The method of claim 88, wherein the cells selected in the positive selection step (i) have at least a 1,000-fold higher expression of endogenous Tgdf1 as compared to the cells not selected.

91.-103. (canceled)

104. The method of claim 88, wherein the cells selected in the selection step (ii) have a lower expression of at least 2 of the following genes; endogenous Oct4, endogenous Nanog, endogenous Rex1, endogenous Tdgf1 by a statistically significant amount relative to the cells not selected.

105.-113. (canceled)
